(19) United States
(12) Patent Application Publication
(10) Pub. No.: US 2026/0272270 A1
(43) Pub. Date: Sep. 17, 2026

(54) CONTROL DEVICES WITH LOCKING HANDLE

(71) Applicant: Boston Scientific Scimed, Inc., Maple Grove, MN (US)

(72) Inventors: Scott E. Brechbiel, Johnston, RI (US); Julie Gagnon, Providence, RI (US); Andrew James Schaubhut, Bolton, MA (US); Dov Goldvasser, Framingham, MA (US)

(73) Assignee: Boston Scientific Scimed, Inc., Maple Grove, MN (US)

(21) Appl. No.: 19/564,567

(22) Filed: Mar. 12, 2026

Related U.S. Application Data
(60) Provisional application No. 63/770,556, filed on Mar. 12, 2025.

(51) Int. Cl. A61B 1/00; A61B 1/018

(52) U.S. Cl. CPC A61B 1/00133; A61B 1/0014; A61B 1/018

(57) ABSTRACT

A medical device system may include a handle having first and second portions, with the first portion being axially displaceable relative to the second portion. One or more snap members may extend from the body member, each having a radially inwardly extending protrusion. The second portion may include a plurality of recesses configured to selectively receive the radially inwardly extending protrusions of the snap members, enabling selective locking engagement between the first and second portions.

### CROSS-REFERENCE TO RELATED APPLICATIONS

This application claims the benefit of U.S. Provisional Patent Application Ser. No. 63/770,556 filed on Mar. 12, 2025, the disclosure of which is incorporated herein by reference.

### FIELD

The disclosure pertains to medical devices, and methods for manufacturing and use of these medical devices. More particularly, the present disclosure pertains to medical devices for accessing a body lumen along a biliary and/or pancreatic tract.

### BACKGROUND

During endoscopic procedures, accessory devices may be used along with scope devices. Each of the accessory device and the scope device may have one or more actuators, buttons, or the like to be activated by an operator. Conventionally, different operators have operated the accessory device and the scope device. For example, a physician may actuate, operate, and/or maintain a position of a scope device while a technician may actuate and/or operate an accessory device (e.g., a tome). The use of two operators may lead to, for example, delayed responses, miscues, increased risks, or the like during the procedure. There is an ongoing need to provide alternative configurations of end effectors as well as alternative methods for manufacturing and using medical devices.

### SUMMARY

This disclosure provides design, material, manufacturing method, and use alternatives for medical devices and medical systems.

In an example, a medical device system may include a handle having a first portion and a second portion, where the first portion may be axially displaceable relative to the second portion. The first portion may include a body member having a central lumen extending therethrough and one or more snap members extending from the body member, where each snap member may include a radially inwardly extending protrusion. The second portion may include a plurality of recesses configured to selectively receive the radially inwardly extending protrusions of the one or more snap members.

Alternatively, or additionally, to any of the examples above, in another example, the one or more snap members may include a first snap member and a second snap member.

Alternatively, or additionally, to any of the examples above, in another example, each of the first and second snap members may include a gap positioned radially outward thereof.

Alternatively, or additionally, to any of the examples above, in another example, the radially inwardly extending protrusions may have a V-shaped cross-sectional shape.

Alternatively, or additionally, to any of the examples above, in another example, the radially inwardly extending protrusions may have a hemispherical cross-sectional shape.

Alternatively, or additionally, to any of the examples above, in another example, the second portion may include a slot extending through a thickness thereof and a first rail and a second rail positioned on opposing sides of the slot.

Alternatively, or additionally, to any of the examples above, in another example, the plurality of recesses may be formed in the first and second rails.

Alternatively, or additionally, to any of the examples above, in another example, the plurality of recesses may extend along an entire length of the first and second rails.

Alternatively, or additionally, to any of the examples above, in another example, the first portion may be rotatable between a locked configuration and an unlocked configuration.

Alternatively, or additionally, to any of the examples above, in another example, in the unlocked configuration, the snap members may be circumferentially offset from the plurality of recesses.

Alternatively, or additionally, to any of the examples above, in another example, the first portion may include a proximal body member and a distal body member.

Alternatively, or additionally, to any of the examples above, in another example, the proximal body member may be rotatable relative to the distal body member.

Alternatively, or additionally, to any of the examples above, in another example, the proximal and distal body members may be axially coupled such that axial movement of one of the proximal or distal body members may cause axial movement of the other of the proximal or distal body members.

Alternatively, or additionally, to any of the examples above, in another example, the system may further include an elongate tube having a proximal end and a distal end.

Alternatively, or additionally, to any of the examples above, in another example, the system may further include a wire filament extending through the elongate tube.

In an example, a medical device system may include a handle having a first portion and a second portion, where the first portion may be axially displaceable relative to the second portion. The first portion may include a body member having a central lumen extending therethrough and one or more snap members extending from the body member, where each snap member may include a radially inwardly extending protrusion. The second portion may include a plurality of recesses configured to selectively receive the radially inwardly extending protrusions of the one or more snap members.

Alternatively, or additionally, to any of the examples above, in another example, the one or more snap members may include a first snap member and a second snap member.

Alternatively, or additionally, to any of the examples above, in another example, each of the first and second snap members may include a gap positioned radially outward thereof.

Alternatively, or additionally, to any of the examples above, in another example, the protrusions may have a V-shaped cross-sectional shape.

Alternatively, or additionally, to any of the examples above, in another example, the protrusions may have a hemispherical cross-sectional shape.

Alternatively, or additionally, to any of the examples above, in another example, the second portion may include a slot extending through a thickness thereof and a first rail and a second rail positioned on opposing sides of the slot.

Alternatively, or additionally, to any of the examples above, in another example, the plurality of recesses may be formed in the first and second rails.

Alternatively, or additionally, to any of the examples above, in another example, the plurality of recesses may extend along an entire length of the first and second rails.

Alternatively, or additionally, to any of the examples above, in another example, the first portion may be rotatable between a locked configuration and an unlocked configuration.

Alternatively, or additionally, to any of the examples above, in another example, in the unlocked configuration, the one or more snap members may be circumferentially offset from the plurality of recesses.

In an example, a medical device system may include a handle having a first portion and a second portion, where the first portion may be axially displaceable relative to the second portion. The first portion may include a proximal body member and a distal body member, where the proximal body member may be rotatable relative to the distal body member, and where the proximal and distal body members may be axially coupled such that axial movement of one of the proximal or distal body members may cause axial movement of the other of the proximal or distal body members.

Alternatively, or additionally, to any of the examples above, in another example, the proximal body member may include a recess and the distal body member may include a protrusion configured to be received within the recess.

Alternatively, or additionally, to any of the examples above, in another example, the proximal body member may include one or more cantilevered snap members.

Alternatively, or additionally, to any of the examples above, in another example, the second portion may include a plurality of recesses configured to selectively receive protrusions of the one or more cantilevered snap members.

Alternatively, or additionally, to any of the examples above, in another example, the proximal body member may be rotatable between a locked configuration where the protrusions may be received within the plurality of recesses and an unlocked configuration where the protrusions may be circumferentially offset from the plurality of recesses.

In an example, a medical device system may include an elongate tube having a proximal end and a distal end, a wire filament extending through the elongate tube, and a handle coupled to the proximal end of the elongate tube. The handle may include a first portion and a second portion, where the first portion may be axially displaceable relative to the second portion. The first portion may include one or more snap members, and the second portion may include a plurality of recesses configured to selectively receive protrusions of the one or more snap members.

Alternatively, or additionally, to any of the examples above, in another example, the first portion may include a proximal body member and a distal body member.

Alternatively, or additionally, to any of the examples above, in another example, the proximal body member may be rotatable relative to the distal body member.

Alternatively, or additionally, to any of the examples above, in another example, the proximal and distal body members may be axially coupled.

Alternatively, or additionally, to any of the examples above, in another example, the first portion may be rotatable between a locked configuration where the protrusions may be received within the plurality of recesses and an unlocked configuration where the protrusions may be circumferentially offset from the plurality of recesses.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 is a schematic view of an illustrative system for accessing an opening of a body lumen of a subject;

FIG. 2 is a schematic view of the system depicted in FIG. 1, taken from circle-2;

FIG. 3 is a schematic view of an illustrative system for accessing a body lumen;

FIG. 4 is a schematic side view of an illustrative handle that may be used to actuate and mechanically lock a wire filament of an accessory device;

FIG. 5 is a cross-sectional view of the illustrative handle of FIG. 4 taken at line 5-5.

FIG. 6 is a schematic cross-sectional view of another illustrative locking mechanism;

FIG. 7 is a schematic cross-sectional view of another illustrative locking mechanism;

FIG. 8 is a schematic side view of another illustrative handle that may be used to actuate and mechanically lock a wire filament of an accessory device;

FIG. 9 is a cross-sectional view of the illustrative handle of FIG. 8, taken at line 9-9 of FIG. 8;

FIG. 10 is a cross-sectional view of the illustrative handle of FIG. 8, taken at line 10-10 of FIG. 8;

FIG. 11 is a cross-sectional view of the illustrative handle of FIG. 8, taken at line 11-11 of FIG. 8;

FIG. 12A is a partial cross-section of an alternative locking member, in a first, unlocked configuration;

FIG. 12B is a partial cross-section of the illustrative locking member of FIG. 12A in a second, locked configuration;

FIG. 13 is a schematic perspective view of another illustrative handle that may be used to actuate and mechanically lock a wire filament of an accessory device.

FIG. 14A is a cross-sectional view of the illustrative handle of FIG. 13, taken at line 14A-14A of FIG. 13 with a locking member in a first, unlocked, configuration;

FIG. 14B is the cross-sectional view of FIG. 14A with the locking member in a second, locked, configuration;

FIG. 15 is a perspective view of another illustrative locking mechanism;

FIG. 16 is a cross-sectional view of the illustrative locking mechanism of FIG. 15, taken at line 16-16 of FIG. 15;

FIG. 17 is a perspective view of another illustrative handle, in a second, or locked configuration, that may be used to actuate and mechanically lock a wire filament of an accessory device;

FIG. 18A is a cross-sectional view of the illustrative handle of FIG. 17, taken at line 18A-18A of FIG. 17;

FIG. 18B is a cross-sectional view of the illustrative handle of FIG. 17 in a first, or unlocked, configuration;

FIG. 19 is a cross-sectional view of the handle of FIG. 17, taken at line 19-19 of FIG. 17.

FIG. 20 is a perspective view of a partial cross-section of another illustrative handle that may be used to actuate and mechanically lock a wire filament of an accessory device;

FIG. 21 is a top view of the portion of the illustrative handle of FIG. 20;

FIG. 22 is an exploded perspective view of the illustrative shuttle, wire filament and spring member of FIG. 20;

FIG. 23 is a schematic perspective view of another illustrative handle that may be used to actuate and mechanically lock a wire filament of an accessory device;

FIG. 24 is a cross-sectional view of the illustrative handle of FIG. 23, taken at line 24-24 of FIG. 23;

FIG. 25A is a cross-sectional view of the illustrative handle of FIG. 23, taken at line 25A-25A of FIG. 23 with a locking member in a second, locked, configuration;

FIG. 25B is the cross-sectional view of FIG. 25A with the locking member in a first, unlocked, configuration;

FIG. 26 is a schematic perspective view of another illustrative handle that may be used to actuate and mechanically lock a wire filament of an accessory device;

FIG. 27 is a cross-sectional view of the illustrative handle of FIG. 26, taken at line 27-27 of FIG. 26;

FIG. 28A is a cross-sectional view of the illustrative handle of FIG. 26, taken at line 28A-28A of FIG. 26 with a locking member in a first, unlocked, configuration;

FIG. 28B is the cross-sectional view of FIG. 28A with the locking member in a second, locked, configuration;

FIG. 29 is a schematic perspective view of another illustrative handle that may be used to actuate and mechanically lock a wire filament of an accessory device;

FIG. 30 is a cross-sectional view of the illustrative handle of FIG. 29, taken at line 30-30 of FIG. 29;

FIG. 31A is a schematic distal perspective view of another illustrative handle that may be used to actuate and mechanically lock a wire filament of an accessory device, in a first or unlocked configuration;

FIG. 31B is a schematic distal perspective view of the illustrative handle of FIG. 31A, in a second or locked configuration;

FIG. 32 is a schematic partial cross-sectional view of the illustrative handle of FIG. 31A in the second or locked configuration;

FIG. 33 is a side view of another illustrative handle that may be used to actuate and mechanically lock a wire filament of an accessory device;

FIG. 34 is a cross-sectional view of the illustrative handle of FIG. 33, taken at line 34-34 of FIG. 33.

FIG. 35 is a cross-sectional view of the handle of FIG. 33 having an alternative arrangement;

FIG. 36 is a schematic perspective view of another illustrative handle that may be used to actuate and mechanically lock a wire filament of an accessory device;

FIG. 37 is a cross-sectional view of the illustrative handle of FIG. 36, taken at line 37-37 of FIG. 36;

FIG. 38A is a cross-sectional view of the illustrative handle of FIG. 36, taken at line 38A-38A of FIG. 36;

FIG. 38B is the cross-sectional view of FIG. 38A with the handle in an unlocked configuration;

FIG. 39 is a schematic side view of another illustrative handle that may be used to actuate and mechanically lock a wire filament of an accessory device;

FIG. 40A is a cross-sectional view of the illustrative handle of FIG. 39, taken at line 40A-40A of FIG. 39;

FIG. 40B is a cross-sectional view of the illustrative handle of FIG. 39, taken at line 40B-40B of FIG. 39;

FIG. 41A is a partial cross-sectional view of another illustrative handle in a first locked configuration that may be used to actuate and mechanically lock a wire filament of an accessory device; and

FIG. 41B is a partial cross-sectional view of the handle of FIG. 41A in a second unlocked configuration.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Although configurations of the present disclosure may be described with specific reference to medical devices and systems (e.g., endoscopic devices, accessory tools, and/or guidewires inserted through a duodenoscope, near or through a papilla, or the like) for selective access to, aligning with, and/or cannulation of the common bile duct (CBD) or pancreatic duct (PD) during an Endoscopic Retrograde Cholangiopancreatography (ERCP) procedure, it should be appreciated that such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may be inserted via different access points and approaches, e.g., percutaneously, endoscopically, laparoscopically, or some combination thereof.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it would be within the knowledge of one skilled in the art to effect the particular feature, structure, or characteristic in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example configurations of the disclosure.

Current procedures using an endoscope, duodenoscope, or a colonoscope, often use the services of an additional professional to operate the end effector. An end effector may be an operational tool or components used with an endoscopic device to perform a specific action during a procedure. In some cases, the physician may be focused on the operation of the scope to maintain position while the technician/nurse operates the action of the accessory device (e.g., in some cases with the end effector). Verbal communication, physician to technician, may be required to achieve the performance and/or desired results. Often this communication can be tedious and may not result in the exact actions desired or may increase time to perform the desired result. In addition, during a procedure a physician's left-hand may control rotation of the scope shaft, distal articulation, locking mechanisms, and/or air, water, and suction control while the right hand may perform axial movement of the device shaft. Thus, it may be desirable for any additional functions to be performed by the physician to be configured to be performed with the right hand.

A sphincterotome is a device used to cannulate the papilla during endoscopic retrograde cholangiopancreatography (ERCP) procedures. The sphincterotome is steerable via a wire attached to a sliding handle component. “Bowing” refers to the movement to bend the tip of the sphincterotome by moving a sliding handle component. The present disclosure is directed towards systems and method for locking the moving portion of the handle relative to the stationary portion. This would maintain the bow shape that the physician desires for a given procedure without requiring the physician to hold the sliding handle component in the desired position. Further, while the systems, devices, and methods described herein, are discussed with respect to a sphincterotome, the systems, devices, and methods may be used in other steerable medical devices.

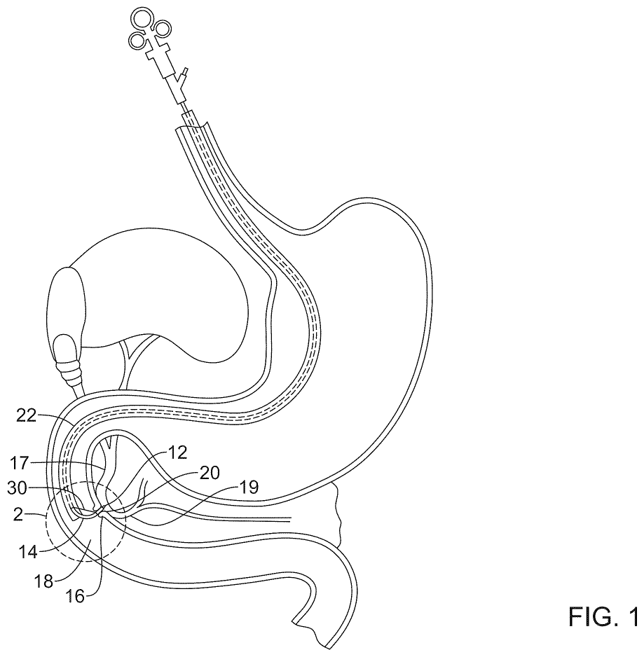

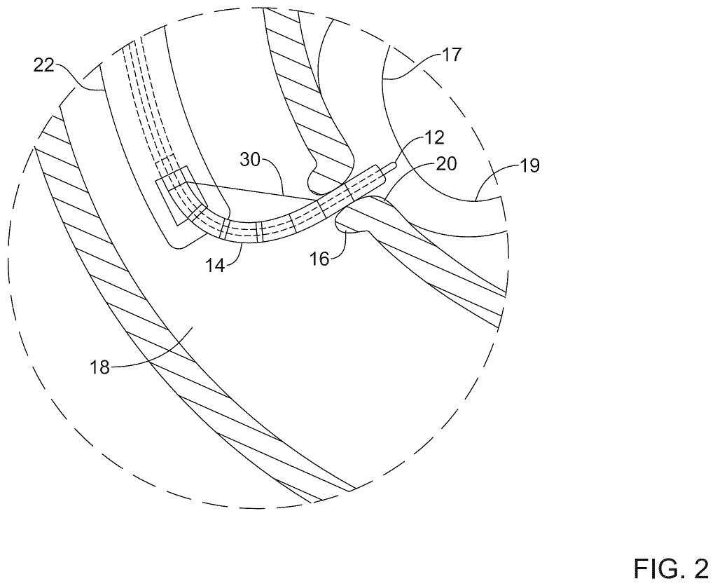

FIGS. 1 and 2 depict a selective cannulation during an ERCP procedure, which includes a guidewire 12 and/or an accessory medical device 14 (e.g., an endoscopic accessory device, such as a sphincterotome, and/or other suitable accessory medical device) being passed towards, against, and/or through a body lumen such as the major papilla 16 (e.g., ampullary entry) near the descending duodenum 18 to access the Sphincter of Oddi Complex 20. During the cannulation procedure, a distal portion of a medical device 22 (e.g., an endoscope, duodenoscope, guide catheter, etc.) may be positioned within the descending duodenum 18. The guidewire 12 and the accessory medical device 14 may be advanced through a working channel (e.g., a lumen) of the medical device 22 towards the major papilla 16. Additionally, the guidewire 12 and/or the accessory medical device 14 may be advanced against and/or through the major papilla 16 to one of the common bile duct 17 and the pancreatic duct 19.

Accessing the papilla 16, the common bile duct 17, and/or the pancreatic duct 19 may be difficult because the openings are small compared to a size (e.g., a diameter or other size measurement) of many accessory medical devices, the openings may be closely located, the openings may be completely collapsed/closed, the openings may extend into the descending duodenum 18 at an angle that may be difficult to visualize and/or access, and/or it may be difficult to control a position of a distal tip of the accessory medical device 14. Thus, a medical professional may be required to manipulate the accessory medical device 14 and guidewire 12 by manually rotating the medical device 22, pulling on a wire filament 30 to adjust the accessory medical device 14 in a single direction, and/or use an elevator and/or other suitable ramped surface within the distal end of the medical device 22 in an attempt to better align or orient the accessory medical device 14 and/or the guidewire 12 with respect to the medical device 22 and the openings of the papilla 16, the common bile duct 17, and/or the pancreatic duct 19.

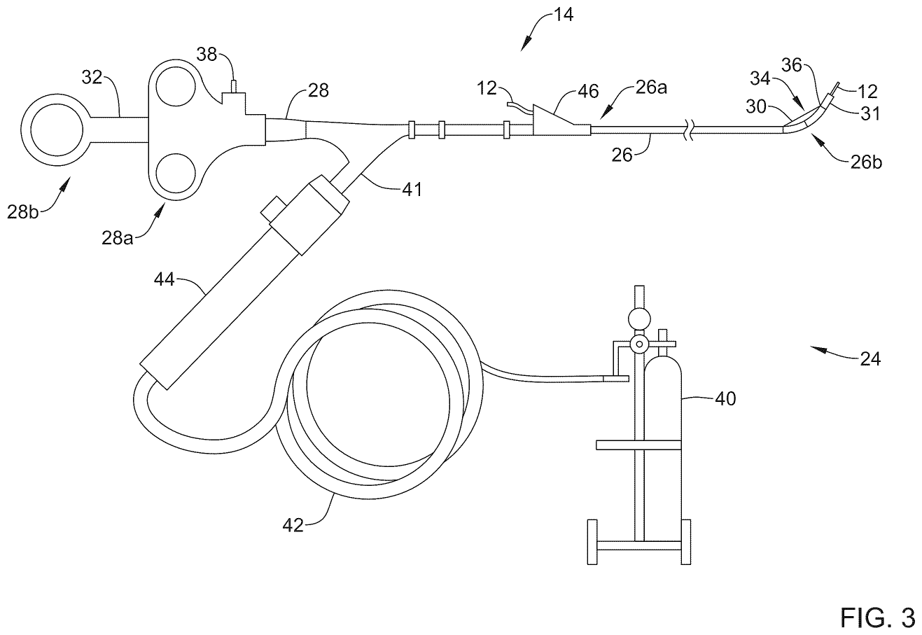

FIG. 3 schematically depicts an illustrative system 24 for accessing a body lumen. In some cases, the system 24 may include, among other components, the accessory medical device 14 (such as, but not limited to, a sphincterotome) with a flexible elongate tube 26. The tube 26 may have a proximal end 26a and a distal end 26b. The distal end 26b of the tube 26 may have a distal tip 31 extending distal of a distal end 36 of the wire filament 30 and may be configured to be directed toward and/or through an opening of a body lumen. The proximal end 26a of the tube 26 may be connected to, coupled with, or otherwise in communication with a handle 28.

The handle 28 may be any suitable type of handle. In one example, the handle 28 may have a first portion 28a configured to receive fingers (e.g., receive two fingers) of a user using the accessory medical device 14 to hold the accessory medical device 14 and a second portion 28b configured to receive a thumb of the user. In some cases, the second portion 28b of the handle 28 may be an actuating portion 32 of the handle and a user may adjust the second portion 28b relative to the first portion 28a to control positioning of a distal end of the accessory medical device 14, as discussed herein or otherwise. Although not required, the handle 28, as illustrated, may be held and/or operated with a single hand of a user.

The second portion 28b of the handle 28 may be configured to be adjusted or manipulated to control a position of a distal end 26b of the elongate tube 26 and/or the wire filament 30. In some cases, the second portion 28b may be configured to adjust axially or longitudinally and radially or rotationally to adjust a position of the distal end 26b of the elongate tube 26 and/or the wire filament 30 in a plurality of directions.

A wire filament 30 may extend along the elongate tube 26 and may be energized for the purpose of cutting tissue within a subject. The wire filament 30 may have a proximal end (not shown) connected to an actuating portion 32 (e.g., the second portion 28b) of the handle 28, and a proximal portion extending at least partially within the tube 26. The wire filament 30 also may have a distal portion 34 (e.g., a cutting portion) extending external to the elongate tube 26, and a distal end 36 of the wire filament 30 may be connected to the distal end 26b of the elongate tube 26.

The wire filament 30 may be received within a lumen of the tube 26 at least along a proximal portion of the wire filament 30 and may be configured to slide within the lumen. In some cases, the actuating portion 32 of the handle 28 may be coupled to the wire filament 30 and manipulated to slide the wire filament 30 along the tube 26 (e.g., within the lumen of the tube 26). In one example, the actuating portion 32 (e.g., the second portion 28b) of the handle 28 may be longitudinally adjusted relative to the first portion 28a of the handle 28 to apply and remove tension to and/or from the wire filament 30.

Sliding or adjusting the wire filament 30 may result in the distal end 26b of the elongate tube 26 moving responsively to the sliding of the wire filament 30. As such, the actuating portion 32 of the handle 28 may be manipulated to control or adjust a position of the distal end 26b of the elongate tube 26, for example, to direct the distal end 26b toward the opening of a body lumen. The distal tip 31 of the elongate tube may be flexible and configured to adjust in response to steering with or adjustment of a control configuration at a proximal end of the accessory medical device 14. In some cases, natural tension in or acting on the tube 26 and/or the wire filament 30 may result in the wire filament 30 returning to a relaxed position or state once a tension applied to the wire filament 30 via the handle 28 is released. The distal tip 31 may be formed from any suitable material configured to bend, deflect, or otherwise move in response to forces acting thereon. In one example, the distal tip may be formed from a polyether block amide (PEBA, such as, for example, PEBAX®), but this is not required, and other suitable materials are contemplated. Accessory devices having a steerable distal tip are described in commonly assigned U.S. Patent Publication Number 2023/0372007 titled, MEDICAL DEVICE WITH A STEERABLE TIP, the disclosure of which is hereby incorporated by reference.

In some cases, the wire filament 30 may be utilized to electrically cut or remove tissue of the subject (e.g., to cut and cauterize tissue) and may be considered a cautery wire. As such, the handle 28 or other suitable portion of the medical device 22 may include an electrical connection 38 for an energy source (e.g., a radiofrequency energy source, or the like) to energize the wire filament 30 and facilitate cutting tissue. When the actuating portion 32 of the handle 28 is manipulated to adjust a position of the distal end 26b of the tube 26 and/or utilize the wire filament 30 for cutting tissue, the wire filament 30 may be spaced from the tube 26 and electrified to facilitate cutting tissue at or adjacent to the opening of the body lumen.

In some cases, a lumen of the one or more lumens of the elongate tube 26 may have a fluid connection at a proximal end for a fluid source (e.g., a pressurized fluid source 40 such as a CO2 tank, a contrast fluid source such as a syringe, etc.) The lumen may be configured to apply a fluid from the fluid source through an opening at the distal end 26b of the elongate tube 26 to a body lumen of the subject.

Although FIG. 3 depicts the fluid source 40 coupled to a fluid port 41 of the accessory medical device 14 via a fluid tube 42 and a flow regulator 44, the fluid source 40 and/or other suitable fluid sources (e.g., syringes, etc.) may be coupled to the fluid port 41 in other suitable manners. In one example, a syringe with contrast fluid may be coupled to the fluid port 41 via a luer lock connection and/or other suitable connection. Contrast agent may be delivered into the body lumen and may be used to temporarily improve imaging of the inside of the body lumen by, for example, x-ray, computed tomography (CT), or magnetic resonance (MR) imaging, ultrasound, and the like. Alternatively, separate lumens and separate fluid ports may be provided for each of a pressurized fluid and a contrast fluid.

A further lumen of the one or more lumens may be configured to accept the guidewire 12 and/or one or more other suitable medical devices usable with the accessory medical device 14. The further lumen may extend from the distal end 26b of the tube 26 (e.g., an opening at the distal tip 31) at least partially along the tube 26 toward the proximal end 26a of the tube 26.

In some cases, the guidewire 12 or other suitable medical devices may extend through the further lumen of the elongate tube 26 and proximally out of an aperture port 46. The aperture port 46 may be located at the distal end of the handle 28 and/or at one or more other suitable locations. In some cases, the aperture port 46 and the tube 26 may be configured so that the guidewire 12 and/or other suitable medical devices may be stripped through the side of the tube 26 for a rapid removal or exchange of devices, but this is not required.

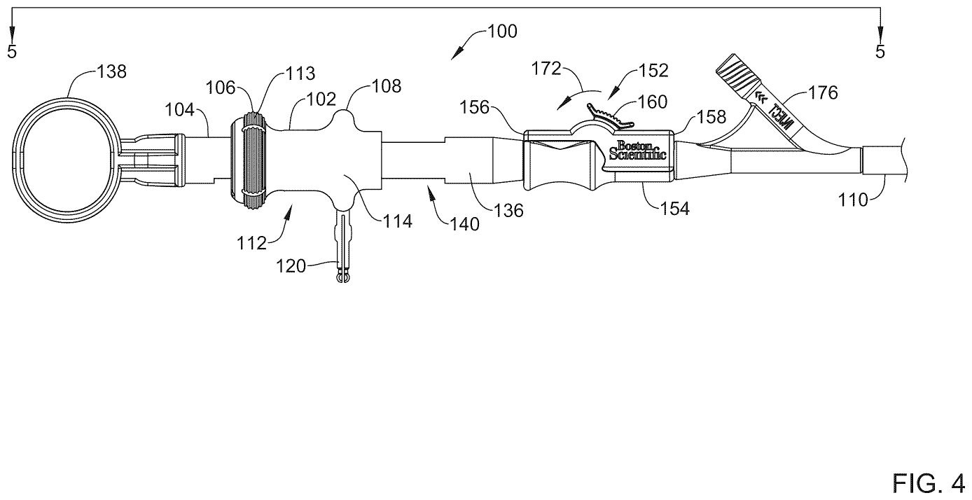

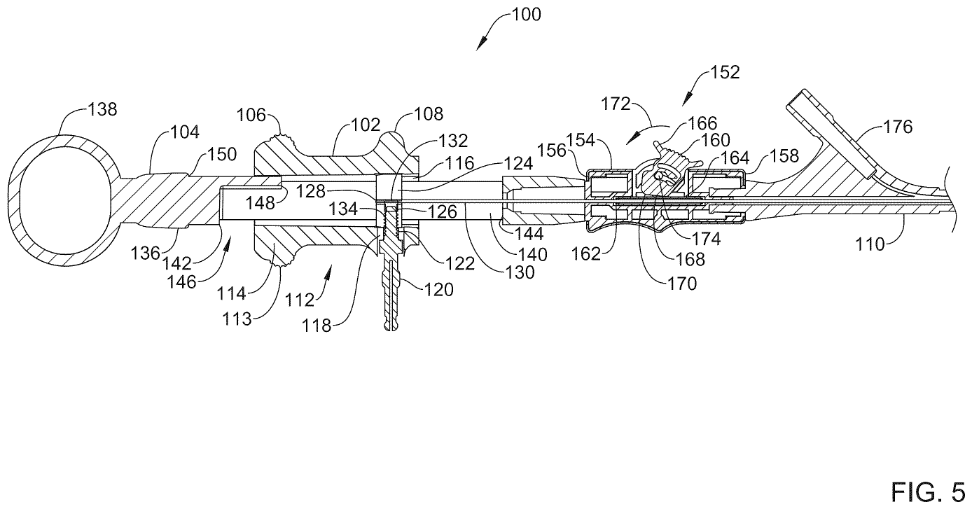

FIG. 4 is a schematic side view of another illustrative handle 100 that may be used to actuate and mechanically lock a wire filament of an accessory device. FIG. 5 is a cross-sectional view of the illustrative handle 100 of FIG. 4, taken at line 5-5 of FIG. 4. The handle 100 may be configured to be coupled to a proximal end of the elongate tube 110 of an accessory device (e.g., similar in form and function to the elongate tube 26 and accessory device 14 described herein). The handle 100 may include a first portion 102 and a second portion 104. The first portion 102 of the handle 100 may be axially displaceable relative to the second portion 104 of the handle 100. For example, the first portion 102 may be proximally retracted relative to the second portion 104 to deflect the distal tip of the elongate tube 110 in a first direction and the first portion 102 may be distally advanced relative to the second portion 104 to deflect the distal tip in a second direction opposite the first direction.

The first portion 102 may include a first, or proximal, gripping region 106 and a second, or distal, gripping region 108. The first and second gripping regions 106, 108 may have an enlarged cross-sectional profile relative to an intermediate region 112 of the first portion 102. This may provide a region for the user to push or pull against to move the first portion 102 relative to the second portion 104. In some cases, one or both of the gripping regions 106, 108 may include texturing 113, such as raised ridges, grooves, bumps, or the like to increase the gripability of the first portion 102.

The first portion 102 may include a generally tubular body portion 114 (e.g., a main body portion) defining a lumen 116 (e.g., a main lumen) extending from a proximal end to a distal end thereof. In some configurations, the body portion 114 may be formed from two or more pieces fixedly or releasably coupled together. The lumen 116 may extend generally coaxially with a proximal end region of the elongate tube 110. The first portion 102 may further include an auxiliary lumen 118 extending generally orthogonal to the main lumen 116. The auxiliary lumen 118 may extend from a laterally outward end to a laterally inward end.

The auxiliary lumen 118 may be configured to receive an electrical connection member 120 therethrough. The connection member 120 may include a threaded region 122 configured to threadably engage a mating threaded region 126 of a mounting member 124. The mounting member 124 may be fixedly disposed within the lumen 116 of the body portion 114. The mounting member 124 may include a first aperture 128 extending from a proximal end to a distal end of the mounting member 124. The first aperture 128 may be configured to receive a proximal end region 132 of a wire filament 130 therethrough. In some cases, the wire filament 130 may be releasably or fixedly secured to the mounting member 124. The mounting member 124 may further include a second aperture 134 extending generally orthogonal to the first aperture 128 and parallel to the auxiliary lumen 118 of the first portion 102 of the handle 100. The threaded region 126 of the mounting member 124 may be formed in the luminal wall of the second aperture 134. The second aperture 134 may be connected to the first aperture 128 to allow a radially inward portion of the connection member 120 to contact the wire filament 130. As the proximal end region 132 of the wire filament 130 is secured to the first portion 102 of the handle 100 via the mounting member 124 and/or the connection member 120, movement of the first portion 102 may be translated to the wire filament 130.

The second portion 104 of the handle 100 may include a generally cylindrical body portion 136 including a ring member 138 extending from a proximal end thereof. The ring member 138 may be configured to receive a thumb or finger of the clinician to hold the second portion 104 relative to the first portion 102 of the handle 100.

The body portion 136 may include a slot 140 extending through a thickness or across a diameter thereof. The slot 140 may extend from a proximal end 142 to a distal end 144. The mounting member 124 of the first portion 102 of the handle 100 may be axially displaceable along the slot 140. In some configurations, the slot 140 may not extend across an entirety of the diameter of the body portion 136 for an entire length of the slot 140. For example, the slot 140 may extend through less than an entirety of the diameter of the body portion 136 along a proximal portion 146 of the slot 140 to define a mechanical stop 148. Said differently, the slot 140 may be configured to create a mechanical stop 148 along a region thereof to limit movement of the first portion 102 of the handle 100. For example, as the first portion 102 is proximally retracted a proximal end of the mounting member 124 may contact the mechanical stop 148 to prevent further proximal movement of the first portion 102. It is contemplated that the length of the slot 140 which does not extend across the entire diameter of the body portion 136 may be selected based on the desired proximal movement of the first portion 102 (and the desired amount of bend at the distal tip). In other examples, the length of proximal retraction of the first portion 102 of the handle 100 may be limited by mechanical engagement of the proximal end of the body portion 114 of the first portion 102 engaging a transition region 150 of the second portion 104. The transition region 150 may be a change in diameter of the second portion 104 from a first diameter to a second, larger, diameter. In some cases, the diameter of the body portion 136 may change. In other examples, the transition region 150 may be a part of the ring member 138.

It is further contemplated that a mechanical stop may be provided to limit distal movement of the first portion 102 of the handle 100. In some cases, the mechanical stop may be the distal end 144 of the slot 140. For example, the distal end of the mounting member 124 may contact the distal end 144 of the slot 140 to limit distal movement of the first portion 102 of the handle 100. In other configurations, the slot 140 may extend through less than an entirety of the diameter of the body portion 136 (similar to proximal portion 146) to define a mechanical stop.

The first portion 102 of the handle 100 may be assembled with the second portion 104 such that the body portion 136 of the second portion 104 is disposed within the lumen 116 of the body portion 114 of the first portion 102. The first portion 102 may be axially displaceable along an outer surface of the second portion 104. The mounting member 124, the connection member 120 and the wire filament 130 may extend into the slot 140 of the second portion 104.

A locking mechanism 152 may be coupled with a distal end of the second portion 104 of the handle 100 using known techniques, such as, but not limited to, press-fits, friction fits, over-molding, heat-shrinking, or the like. The locking mechanism 152 may include a body portion 154 extending from a proximal end 156 to a distal end 158 and a switch or lever 160. A lumen 162 may extend from the proximal end 156 to the distal end 158 of the body portion 154. A flexible tube 164 may be disposed within the lumen 162 of the body portion 154 adjacent to the lever 160. A portion of the wire filament 130 may extend through a lumen of the flexible tube 164.

The lever 160 may include an actuation portion 166 exterior to the body portion 154 and a gripping portion 168 extending radially inwards into the body portion 154. The actuation portion 166 may be movable or actuated by the user. While the actuation portion 166 is illustrated as a switch, the actuation portion 166 may take other forms as desired, such as, but not limited to, a twist knob. The lever 160 may be movable between a first configuration (shown in FIG. 4 and FIG. 5) configured to allow free movement of the wire filament 130 within the flexible tube 164 and a second configuration (not explicitly shown) configured to frictionally engage a luminal surface of the flexible tube 164 with an outer surface of the wire filament 130 to prevent axial movement of the wire filament 130. When the lever 160 is in the first configuration, the gripping portion 168 thereof is radially spaced from an outer surface of the flexible tube 164. In this configuration, the gripping portion 168 does not apply any radially compressive forces to the flexible tube 164 such that the flexible tube 164 is at its maximum luminal diameter. The luminal diameter of the flexible tube 164 may be selected to allow the wire filament 130 to freely move when the lever 160 is in the first configuration. For example, the luminal diameter of the flexible tube 164 may be greater than the outer diameter of the wire filament. However, this is not required.

To move the lever 160 to the second, locked, configuration, the user may exert a generally proximal force, as shown at arrow 172, to the actuation portion 166. This may cause the lever 160 to pivot along a pin 174 to bring a cam surface 170 of the gripping portion 168 into contact with the outer surface of the flexible tube 164. The cam surface 170 may be sized and shaped to exert a radially compressive force onto the outer surface of the flexible tube 164 causing the flexible tube 164 to compress around, pinch, or otherwise contact an outer surface of the wire filament 130. In the collapsed configuration, the luminal surface of the flexible tube 164 frictionally engages the outer surface of the wire filament 130 to prevent axial movement of the wire filament 130. Moving the lever 160 to the locked configuration may allow the user to secure the distal tip of the elongate tube 110 in the desired configuration without requiring the user to hold the first portion 102 of the handle 100 to maintain the desired configuration of the distal tip. To release the distal tip, the actuation portion 166 may be distally advanced to return the gripping portion 168 to a spaced configuration with the flexible tube 164. As the compressive force is removed from the flexible tube 164, the flexible tube 164 may assume an expanded, unbiased configuration allowing free movement of the wire filament 130.

It is contemplated that the movement required to move the actuation portion 166 of the lever 160 may be determined based on the style of the actuation portion 166. For example, a knob may be rotated to move a gripping portion towards or away from the flexible tube 164. While the locking mechanism 152 is illustrated as disposed between the distal end of the second portion 104 of the handle 100 and the elongate tube 110, the locking mechanism 152 may be positioned at other locations, as desired. In some cases, the locking mechanism 152 may be formed with or incorporated into the first portion 102 of the handle 100. In yet other examples, the locking mechanism 152 may be formed with or incorporated into the second portion 104 of the handle 100.

A fluid port 176 may be coupled with the locking mechanism 152 using known techniques, such as, but not limited to, press-fits, friction fits, over-molding, heat-shrinking, or the like. The proximal end of the elongate tube 110 may be coupled with the fluid port 176 using known techniques, such as, but not limited to, press-fits, friction fits, over-molding, heat-shrinking, mechanical coupling members such as pins, or the like. In some cases, the fluid port 176 may be coupled with the second portion 104 of the handle 100.

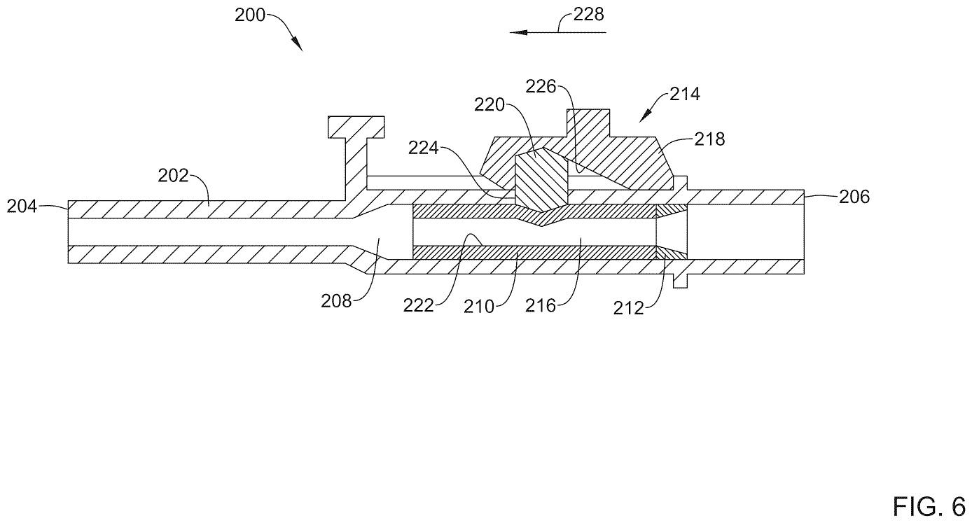

FIG. 6 is a schematic cross-sectional view of an illustrative locking mechanism 200 that may be used in place of the locking mechanism 152 of FIGS. 4 and 5. The locking mechanism 200 may be coupled with a distal end of the second portion of the handle using known techniques, such as, but not limited to, press-fits, friction fits, over-molding, heat-shrinking, or the like. The locking mechanism 200 may include a body portion 202 extending from a proximal end 204 to a distal end 206 and a switch or lever 214. A lumen 208 may extend from the proximal end 204 to the distal end 206 of the body portion 202. A flexible tube 210 may be disposed within the lumen 208 of the body portion 202 adjacent to the lever 214. A portion of the wire filament may extend through a lumen 216 of the flexible tube 210. In some cases, a plug or retention member 212 may be used to retain the flexible tube 210 within the lumen 208 of the body portion 202. However, this is required.

The lever 214 may include an actuation portion 218 exterior to the body portion 202 and an actuation member 220 configured to extend radially inwards of the body portion 202. For example, the actuation member 220 may extend radially inwards through a side opening 224 extending through a thickness of the body portion 202. A longitudinal axis of the actuation member 220 may extend generally orthogonal to a longitudinal axis of the body portion 202. The actuation portion 218 may be movable or actuatable by the user. While the actuation portion 218 is illustrated as a lever or switch, the actuation portion 218 may take other forms as desired, such as, but not limited to, a twist knob. The lever 214 may be movable between a first configuration (shown in FIG. 6) configured to allow free movement of a wire filament within the lumen 216 of the flexible tube 210 and a second configuration (not explicitly shown) configured to frictionally engage a luminal surface 222 of the flexible tube 210 with an outer surface of the wire filament to prevent axial movement of the wire filament. When the lever 214 is in the first configuration, the actuation member 220 thereof is radially spaced from an outer surface of the flexible tube 210 or positioned to apply a radially inward force insufficient to bias the luminal surface of the flexible tube 210 into contact with the outer surface of the wire filament. In this configuration, the actuation member 220 does not apply any radially compressive forces to the flexible tube 210 such that the flexible tube 210 is at its maximum luminal diameter or a luminal diameter greater than an outer diameter of the wire filament. The luminal diameter of the flexible tube 210 may be selected to allow the wire filament to freely move when the lever 214 is in the first configuration. For example, the luminal diameter of the flexible tube 210 may be greater than the outer diameter of the wire filament. However, this is not required.

To move the lever 214 to the second, locked, configuration, the user may exert a generally proximal force, as shown at arrow 228, to the actuation portion 218. This may cause an angled surface 226 of the actuation portion 218 to exert a radially inwards force on the actuation member 220. For example, the angled surface 226 may taper in the distal direction such that a thickness of the actuation portion 218 is at a minimum adjacent to a proximal end of the angled surface 226 and at a maximum adjacent to a distal end of the angled surface 226. As the actuation portion 218 is proximally actuated, the actuation member 220 moves radially inwards. The actuation member 220 may be sized and shaped to exert a radially compressive force onto the outer surface of the flexible tube 210 causing the flexible tube 210 to compress around, pinch, or otherwise contact an outer surface of the wire filament. In the collapsed configuration, the luminal surface 222 of the flexible tube 210 frictionally engages the outer surface of the wire filament to prevent axial movement of the wire filament. In some cases, the angled surface 226 may include a mechanical stop or engagement feature configured to retain the lever 214 in the locked configuration. Moving the lever 214 to the locked configuration may allow the user to secure the distal tip of the elongate tube in the desired configuration without requiring the user to hold the first portion of the handle to maintain the desired configuration of the distal tip. To release the distal tip, the actuation portion 218 may be distally advanced to return the actuation member 220 to a spaced configuration or less radially compressed configuration with the flexible tube 210. As the compressive force is removed from the flexible tube 210, the flexible tube 210 may assume an expanded, unbiased (or less biased) configuration allowing free movement of the wire filament.

It is contemplated that the movement required to move the actuation portion 218 of the lever 214 may be determined based on the style of the actuation portion 218. For example, a knob may be rotated to move a gripping portion towards or away from the flexible tube 210. In some cases, the locking mechanism 200 may be disposed between the distal end of the second portion of the handle and the proximal end of the elongate tube. In other examples, the locking mechanism 200 may be positioned at other locations, as desired. In some cases, the locking mechanism 200 may be formed with or incorporated into the first portion of the handle. In yet other examples, the locking mechanism 200 may be formed with or incorporated into the second portion of the handle.

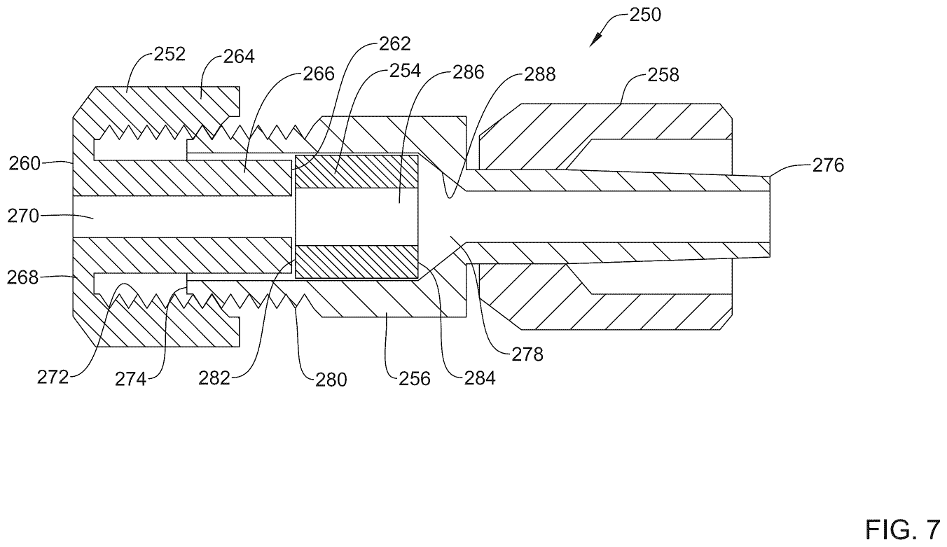

FIG. 7 is a schematic cross-sectional view of another illustrative locking mechanism 250 that may be used in place of the locking mechanism 152 of FIGS. 4 and 5. The locking mechanism 250 may be similar to a Tuohy Borst adaptor or luer lock. The locking mechanism 250 may be coupled with a distal end of the second portion of the handle using known techniques, such as, but not limited to, press-fits, friction fits, over-molding, heat-shrinking, or the like. Generally, the locking mechanism 250 may include a cap 252, a valve or gasket 254, and a body member 256. In some cases, the locking mechanism 250 may include a coupling member 258 configured to couple the locking mechanism 250 to a portion of the handle or the elongate tube.

The cap 252 may extend from a proximal end 260 to a distal end 262. The cap 252 may include an outer coupling member 264 and an inner tubular member 266. The inner tubular member 266 may be coaxially disposed within the outer coupling member 264. Further, the inner tubular member 266 may be coupled to the outer coupling member 264 adjacent the proximal end 260 of the cap 252 via a proximal annular wall 268. A lumen 270 may extend through the inner tubular member 266 from the proximal end 260 to the distal end 262 of the cap 252. The outer coupling member 264 may include a plurality of threads 272 formed on a luminal surface thereof. The threads 272 may be configured to engage mating threads 280 on the body member 256.

The body member 256 may be generally tubular having a lumen 278 extending from a from a proximal end 274 to a distal end 276 thereof. The body member 256 may include a plurality of threads 280 formed in an outer surface thereof. The plurality of threads 280 may extend distally from the proximal end 274 of the body member 256. In some configurations, the distal end region of the body member 256 may have a smaller outer diameter than the proximal end region. However, this is not required. The coupling member 258, if so provided may be coupled, secured, or otherwise affixed to the outer surface of the distal end region of the body member 256.

The gasket 254 may be a generally flexible or compressible tubular member extending from a proximal end 282 to a distal end 284. A lumen 286 may extend from the proximal end 282 to the distal end 284. The gasket 254 may be positioned within the lumen 278 of the body member 256. The distal end 284 of the gasket 254 may be configured to abut or be pushed against an inner wall 288 of the body member 256. The inner wall 288 may be a tapered or have a step-wise transition in inner diameter of the body member 256 from the proximal end region to the distal end region thereof. The distal end 262 of the cap 252 may be configured to selectively abut or contact the proximal end 282 of the gasket 254.

A wire filament, such as the wire filament 130 described herein, may extend through the lumen 270 of the cap 252, the lumen 286 of the gasket 254, and the lumen 278 of the body member 256. In use, the cap 252 may be turned in a first direction to move the distal end 262 thereof in a distal direction. This may exert a distal pushing force on the gasket 254 causing a length of the gasket 254 to decrease as well as reducing the diameter of the lumen 286 of the gasket 254. This may cause the luminal surface of the gasket 254 to come into contact with and frictionally engage an outer surface of the wire filament. The amount of compression exerted on the outer surface of the wire filament may be controlled by how far the cap 252 is distally advanced. For example, the compression exerted on the outer surface of the wire filament may increase as the cap 252 is moved distally. This may allow the user to customize how much friction is applied to the outer surface of the wire filament. For example, the user may rotate the cap 252 to apply an amount of compression or friction that holds the distal tip in the desired configuration while still allowing the wire filament to be actuated with a greater level of force. To release the wire filament, the cap 252 may be rotated in a second direction, opposite the first direction, to move the cap 252 in a proximal direction. As the cap 252 moves proximally, the distal force applied to the proximal end 282 of the gasket 254 may reduce or lessen thus allowing the diameter of the lumen 278 and/or the length of the gasket 254 to expand.

In some cases, the locking mechanism 250 may be disposed between the distal end of the second portion of the handle and the proximal end of the elongate tube. In other examples, the locking mechanism 250 may be positioned at other locations, as desired. In some cases, the locking mechanism 250 may be formed with or incorporated into the first portion of the handle. In yet other examples, the locking mechanism 250 may be formed with or incorporated into the second portion of the handle. It is further contemplated, that while not explicitly shown, the cap 252 may include features configured to facilitate rotatably coupling the cap 252 to the handle or other component of the accessory device.

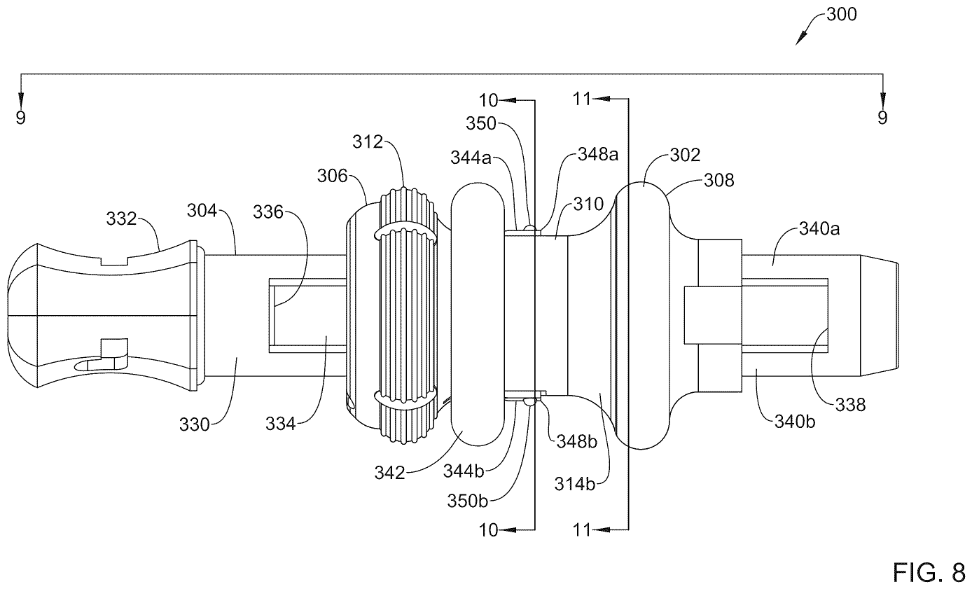

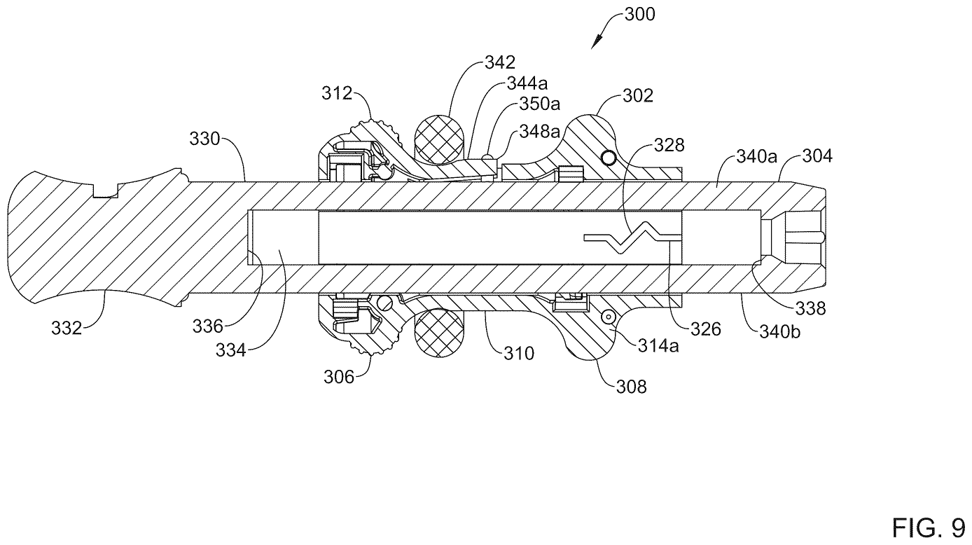

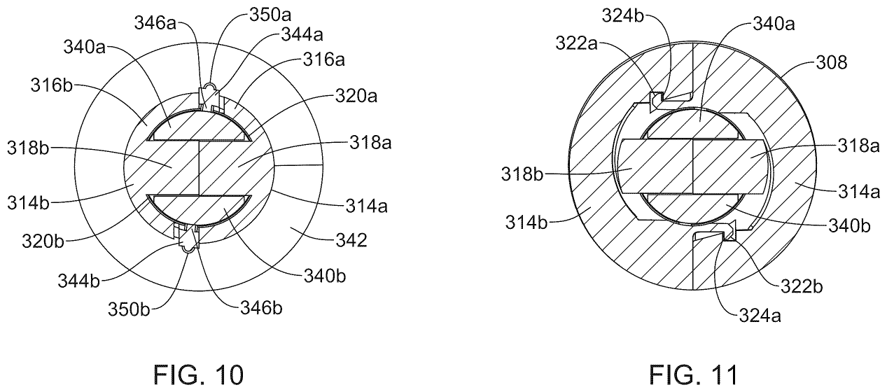

FIG. 8 is a schematic side view of another illustrative handle 300 that may be used to actuate and mechanically lock a wire filament of an accessory device. FIG. 9 is a cross-sectional view of the illustrative handle 300 of FIG. 8, taken at line 9-9 of FIG. 8. FIG. 10 is a cross-sectional view of the illustrative handle 300 of FIG. 8, taken at line 10-10 of FIG. 8. FIG. 11 is a cross-sectional view of the illustrative handle 300 of FIG. 8, taken at line 11-11 of FIG. 8. The handle 300 may be configured to be coupled to a proximal end of the elongate tube (not explicitly shown) of an accessory device (e.g., similar in form and function to the elongate tube 26 and accessory device 14 described herein). The handle 300 may include a first portion 302 and a second portion 304. The first portion 302 of the handle 300 may be axially displaceable relative to the second portion 304 of the handle 300. For example, the first portion 302 may be proximally retracted relative to the second portion 304 to deflect the distal tip of the elongate tube in a first direction and the first portion 302 may be distally advanced relative to the second portion 304 to deflect the distal tip in a second direction opposite the first direction.

The first portion 302 may include a first, or proximal, gripping region 306 and a second, or distal, gripping region 308. The first and second gripping regions 306, 308 may have an enlarged cross-sectional profile relative to an intermediate region 310 of the first portion 302. This may provide a region for the user to push or pull against to move the first portion 302 relative to the second portion 304. In some cases, one or both of the gripping regions 306, 308 may include texturing 312, such as raised ridges, grooves, bumps, or the like to increase the gripability of the first portion 302.

The first portion 302 may be formed from a first body member 314a and a second body member 314b. The first and second body members 314a, 314b may be fixedly or releasably coupled to one another over the second portion 304 of the handle 300. In some cases, the first and second body members 314a, 314b may be secured using mating latches 322a, 322b and tabs or ridges 324a, 324b, snap fits, friction fits, press-fits, or the like. Each body member 314a, 314b may include an arcuate outer portion 316a, 316b and a radially inward extending portion 318a, 318b. A wall thickness of the arcuate outer portion 316a, 316b may vary along a length thereof. Each of the arcuate outer portions 316a, 316b may have an arc length of approximately 180° such that collectively, the arcuate outer portions 316a, 316b form a complete or substantially complete ring. In some cases, a small gap may be present between adjacent surfaces of the first and second arcuate outer portions 316a, 316b. The radially inwardly extending portions 318a, 318b may extend towards a center of the first portion 302 of the handle 300. In some cases, the radially inwardly extending portions 318a, 318b may contact or meet in the middle. However, this is not required. The radially inwardly extending portions 318a, 318b may extend into a slot 334 of the second portion 304 of the handle 300. One or more gaps or openings 320a, 320b may be formed between the first and second body members 314a, 314b when they are assembled. These gaps 320a, 320b may extend from a proximal end to a distal end of the first and second body members 314a, 314b. The gaps 320a, 320b may be configured to receive the rails 340a, 340b of the second portion 304 to allow the first portion 302 to be axially displaced relative to the second portion 304. While not explicitly shown, in some configurations, the first portion 302 may further include an auxiliary lumen extending generally orthogonal to a longitudinal axis of the first portion 302. When so provided, the auxiliary lumen may extend from a laterally outward end to a laterally inward end and may be configured to receive an electrical connection member therethrough.

The radially inwardly extending portions 318a, 318b may each include a recess extending proximally from the distal end of the first portion 302 of the handle 300. Collectively, the recesses form a lumen 326 for receiving a proximal end of a wire filament (e.g., similar in form and function to wire filament 130) therein. The lumen 326 may include a helical or bent region 328 configured to facilitate retention of the wire filament within the lumen. Additionally, or alternatively, the wire filament may be secured using means such as, but not limited to, adhesives, friction fits, snap fits, press-fits, or the like. As the proximal end region of the wire filament is secured to the first portion 302 of the handle 300 via the lumen 326, axial movement of the first portion 302 may be translated to the wire filament.

The second portion 304 of the handle 300 may include a generally cylindrical body portion 330 including a gripping region 332 extending from a proximal end thereof. The gripping region 332 may be configured to provide a region for the clinician to hold the second portion 304 relative to the first portion 302 of the handle 300. The body portion 330 may include a slot 334 extending through a thickness or across a diameter thereof. The slot 334 may extend from a proximal end 336 to a distal end 338. On either side of the slot 334, the second portion of the handle 300 may include a first rail 340a and a second rail 340b. The first portion 302 of the handle 300 may be axially displaceable along the rails 340a, 340b with the radially inwardly extending portions 318a, 318b of the first portion 302 of the handle 300 axially displaceable within the slot 334.

The proximal end 336 of the slot 334 may be configured to create a mechanical stop when a proximal end of the radially inwardly extending portions 318a, 318b contacts the proximal end 336 of the slot 334 to limit proximal movement of the first portion 302 of the handle 300. Similarly, the distal end 338 of the slot 334 may be configured to create a mechanical stop when a distal end of the radially inwardly extending portions 318a, 318b contacts the distal end 338 of the slot 334 to limit distal movement of the first portion 302 of the handle 300. It is contemplated that the length of the slot 334 may be selected based on the desired proximal and/or distal movement of the first portion 302 (and the desired amount of bend at the distal tip).

The first portion 302 of the handle 300 may be assembled with the second portion 304 such that the body portion 330 (e.g., rails 340a, 340b) of the second portion 304 is disposed within the openings 320a, 320b formed by the first and second body members 314a, 314b of the first portion 302. The first portion 302 may be axially displaceable along an outer surface of the second portion 304.

A movable ring 342 may be movably disposed over the outer surface of the first portion 302 of the handle 300. In the illustrated configuration, the body of the movable ring 342 may have a generally circular cross-section. However, this is not required. The movable ring 342 may be positioned around the intermediate region 310 of the first portion 302 of the handle 300 between the first gripping region 306 and the second gripping region 308. The first and second gripping regions 306, 308 may be sized to prevent the movable ring 342 from being slid or moved proximally beyond a proximal end of the first portion 302 or distally beyond a distal end of the first portion 302. The movable ring 342 may be formed from a flexible material, such as, but not limited to, rubber, silicone, or the like, to allow the movable ring 342 to be positioned over the outer surface of the first portion 302. It is contemplated that the movable ring 342 may be slid, rolled, or otherwise axially displaced to selectively engage one or more deflectable latches or tabs 344a, 344b formed in a side wall of the intermediate region 310 of the first portion 302.

The one or more deflectable tabs 344a, 344b may be coupled with the side wall of the intermediate region 310 along one edge thereof and free from securement along the remaining edges thereof. In the illustrated embodiment, the one or more deflectable tabs 344a, 344b may have a generally rectangular cross-sectional shape in which three edges thereof are free from securement to or are spaced from a remainder of the side wall of the intermediate region 310. The one or more deflectable tabs 344a, 344b may have other cross-sectional shapes, as desired. Each of the one or more deflectable tabs 344a, 344b may include a radially inwardly extending member 346a, 346b. The radially inwardly extending member 346a, 346b may extend towards and contact the outer surface of the rails 340a, 340b of the second portion 304.

The one or more deflectable tabs 344a, 344b may be movable from a first unlocked configuration in which friction between the radially inwardly extending member 346a, 346b and the rails 340a, 340b is at a minimum. In the first unlocked configuration, the first portion 302 of the handle 300 (and thus the wire filament) may be axially displaceable relative to the second portion 304 of the handle 300. When the distal tip of the elongate shaft is in the desired configuration, the movable ring 342 may be slid, rolled, or otherwise axially displaced (proximally or distally depending on a starting position of the movable ring 342) to position the movable ring 342 over the free ends 348a, 348b of the one or more deflectable tabs 344a, 344b to a second, locked configuration. The free ends 348a, 348b may include a raised region 350a, 350b. The raised regions 350a, 350b may increase the outer diameter of intermediate region 310 of the first portion 302 at the free ends 348a, 348b of the one or more deflectable tabs 344a, 344b. The outer diameter of the intermediate region 310 at the raised regions 350a, 350b may be greater than an inner diameter or luminal diameter of the movable ring 342. As the movable ring 342 is moved over the raised regions 350a, 350b, the movable ring 342 may exert a radially inwards biasing force on the one or more deflectable tabs 344a, 344b causing the one or more deflectable tabs 344a, 344b to deflect radially inwards. This may increase the frictional force between the radially inwardly extending members 346a, 346b and the outer surface of the rails 340a, 340b to prevent movement of the first portion 302 of the handle 300 relative to the second portion 304 of the handle 300. In some cases, the raised regions 350a, 350b may include a groove (e.g., extending generally orthogonal to a longitudinal axis of the first portion 302), one or more recesses, or the like to assist in maintaining the position of the movable ring 342 over the free end 348a, 348b of the one or more deflectable tabs 344a, 344b. To release the distal tip, the movable ring 342 may be proximally retracted or distally advanced to remove the biasing force from the one or more deflectable tabs 344a, 344b and reduce or remove the frictional force between the radially inwardly extending members 346a, 346b and the outer surface of the rails 340a, 340b.

The first portion 302 of the handle 300 may include any number of deflectable tabs 344a, 344b desired. For example, the first portion 302 may include a single deflectable tab, three deflectable tabs, four deflectable tabs, or more than four deflectable tabs, as desired. In the illustrated configuration, the one or more deflectable tabs 344a, 344b may be uniformly spaced, approximately 180°, from one another. However, this is not required. The one or more deflectable tabs 344a, 344b may be uniformly spaced or eccentrically spaced, as desired. In the illustrated configuration, the free ends 348a, 348b of the one or more deflectable tabs 344a, 344b are positioned at a distal end of the one or more deflectable tabs 344a, 344b. However, this is not required. In some examples, the free ends 348a, 348b of the one or more deflectable tabs 344a, 344b are positioned at a proximal end of the one or more deflectable tabs 344a, 344b. Further, the one or more deflectable tabs 344a, 344b need not have the same orientation of the free end 348a, 348b thereof. It is contemplated that a height and/or surface area of the radially inwardly extending members 346a, 346b and/or the raised regions 350a, 350b may be increased or decreased to control the amount of force applied to the outer surface of the second portion 304 of the handle 300.

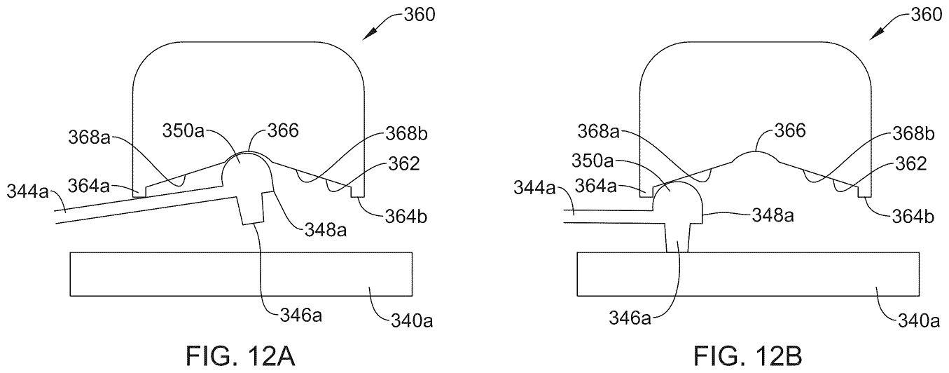

FIG. 12A is a partial cross-section of an alternative locking member 360 that may be used in place of the movable ring of FIGS. 8-11, in a first, unlocked configuration. FIG. 12B is a partial cross-section of the illustrative locking member 360 of FIG. 12A in a second, locked configuration. FIGS. 12A and 12B illustrate one of the deflectable tabs 344a shown in FIGS. 8-11. However, it should be understood that the locking member 360 may engage a second deflectable tab 344b (or any additional deflectable tabs) in a similar manner to that described with respect to the first deflectable tab 344a.

While not explicitly shown, the locking member 360 may have a generally annular shape configured to extend around an outer surface of intermediate region 310 of the first portion of the handle 300 similar to the movable ring 342. The locking member 360 may include a non-linear inner or luminal surface 362. The non-linear luminal surface 362 may include a proximal tab 364a at a proximal end thereof and a distal tab 364b at a distal end thereof. The proximal and distal tabs 364a, 364b may be sized and shaped to create a mechanical stop between the locking member 360 and the raised region 350a of the deflectable tab 344a. The locking member 360 may have a maximum thickness at the proximal and distal tabs 364a, 364b. The thickness of the locking member 360 may decrease in the distal direction from the proximal tab 364a to a central notch 366 to form a first angled surface 368a. The first angled surface 368a extends radially away from the outer surface of the first portion 302 of the handle 300 in the distal direction. The thickness of the locking member 360 may increase in the distal direction from the central notch 366 to the distal tab 364b to from a second angled surface 368b. The second angled surface 368b extends radially towards from the outer surface of the first portion 302 of the handle 300 in the distal direction. In some configurations, the first and/or second angled surfaces 368a, 368b may be substantially parallel to the longitudinal axis of the first portion 302 of the handle 300.

The non-linear luminal surface 362 may be configured to selectively move the deflectable tab 344a into frictional engagement with the outer surface of the rail 340a of the second portion 304 of the handle 300. In the first, unlocked configuration of FIG. 12A, the locking member 360 is positioned over the deflectable tab 344a such that the raised region 350a is positioned within the central notch 366. In some cases, the central notch 366 may have a profile or shape configured to mate with a profile of the raised region 350a. For example, in the illustrated configuration, the central notch 366 has a curved profile to match the curved profile of the raised region 350a. However, this is not required. When the raised region 350a is positioned within the central notch 366, the radially inwardly extending member 346a is spaced from the outer surface of the rail 340a to allow for free movement of the first portion 302 of the handle 300 and thus the wire filament. In some cases, the deflectable tab 344a may be biased to extend at a non-parallel angle to a longitudinal axis of the first portion 302 of the handle 300 or to be spaced from the outer surface of the rail 340a. However, this is not required.

When the distal tip of the elongate shaft is in the desired configuration, the locking member 360 may be slid or otherwise axially displaced (proximally or distally) to position the proximal or distal tabs 364a, 364b adjacent to the raised region 350a. The increased thickness of the locking member 360 adjacent to the proximal or distal tabs 364a, 364b may deflect the deflectable tab 344a radially inwards, as shown in FIG. 12B. This may increase the frictional force between the radially inwardly extending member 346a and the outer surface of the rail 340a to prevent movement of the first portion 302 of the handle 300 relative to the second portion 304 of the handle 300. To release the distal tip, the movable ring 342 may be proximally retracted or distally advanced to position the raised region 350a within the central notch 366 and remove the biasing force from the deflectable tab 344a.

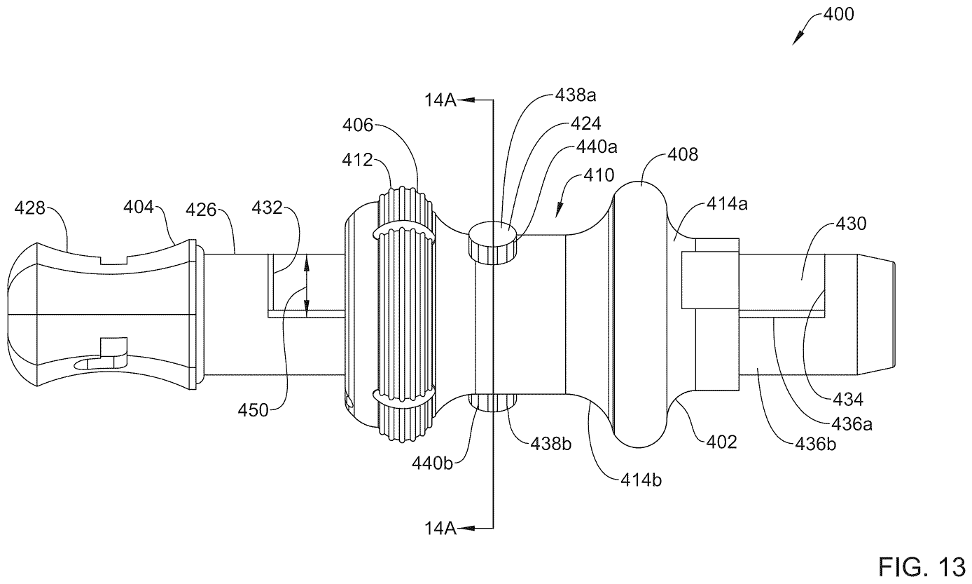

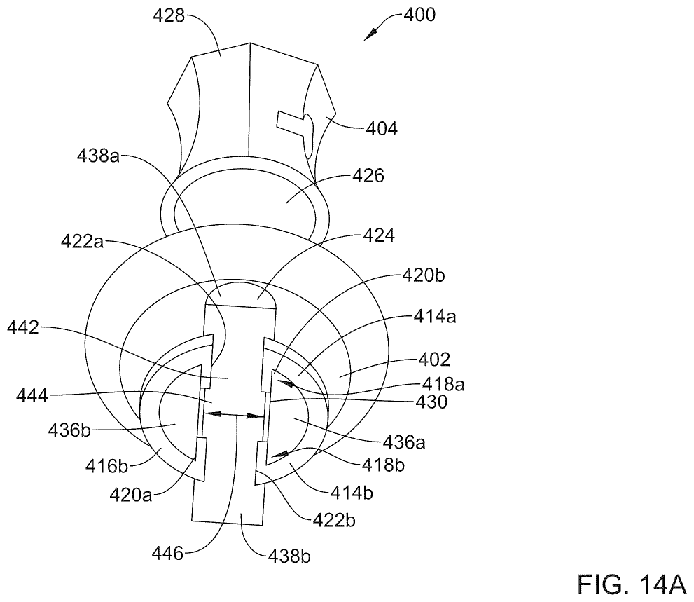

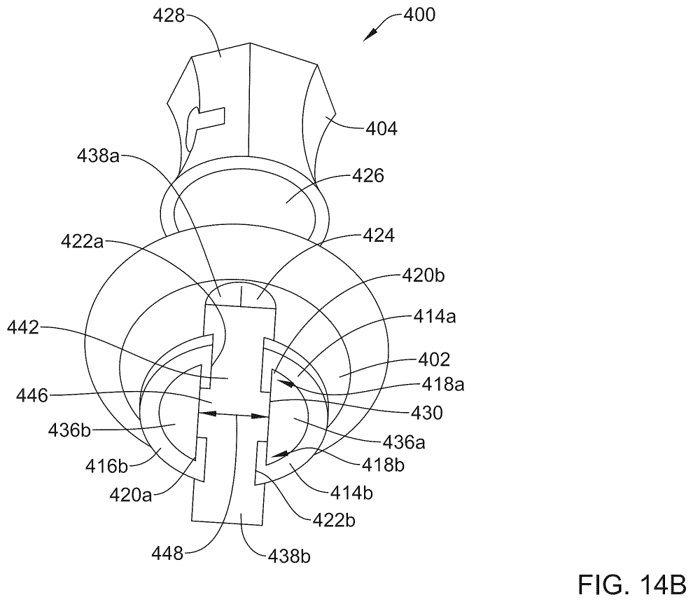

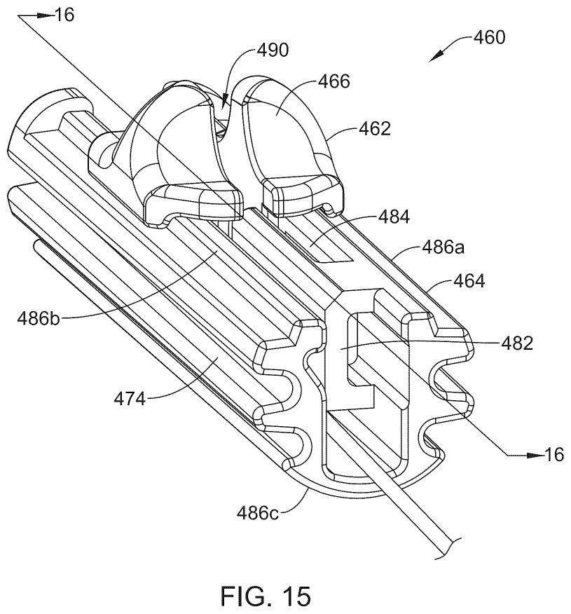

FIG. 13 is a schematic perspective view of another illustrative handle 400 that may be used to actuate and mechanically lock a wire filament of an accessory device. FIG. 14A is a cross-sectional view of the illustrative handle 400 of FIG. 13, taken at line 14A-14A of FIG. 13 with a locking member 424 in a first, unlocked, configuration. FIG. 14B is the cross-sectional view of FIG. 14A with the locking member 424 in a second, locked, configuration. The handle 400 may be configured to be coupled to a proximal end of the elongate tube (not explicitly shown) of an accessory device (e.g., similar in form and function to the elongate tube 26 and accessory device 14 described herein). The handle 400 may include a first portion 402 and a second portion 404. The first portion 402 of the handle 400 may be axially displaceable relative to the second portion 404 of the handle 400. For example, the first portion 402 may be proximally retracted relative to the second portion 404 to deflect the distal tip of the elongate tube in a first direction and the first portion 402 may be distally advanced relative to the second portion 404 to deflect the distal tip in a second direction opposite the first direction.

The first portion 402 may include a first, or proximal, gripping region 406 and a second, or distal, gripping region 408. The first and second gripping regions 406, 408 may have an enlarged cross-sectional profile relative to an intermediate region 410 of the first portion 402. This may provide a region for the user to push or pull against to move the first portion 402 relative to the second portion 404. In some cases, one or both of the gripping regions 406, 408 may include texturing 412, such as raised ridges, grooves, bumps, or the like to increase the gripability of the first portion 402.

The first portion 402 may be formed from a first body member 414a and a second body member 414b. The first and second body members 414a, 414b may be fixedly or releasably coupled to one another over the second portion 404 of the handle 400. In some cases, the first and second body members 414a, 414b may be secured using mating latches and tabs or ridges, snap fits, friction fits, press-fits, or the like. Each body member 414a, 414b may include an arcuate outer portion 416a, 416b and a radially inward extending portion 418a, 418b. A wall thickness of the arcuate outer portion 416a, 416b may vary along a length thereof. Each of the arcuate outer portions 416a, 416b may have an arc length of approximately 180° such that collectively, the arcuate outer portions 416a, 416b form a complete or substantially complete ring. In some cases, a small gap may be present between adjacent surfaces of the first and second arcuate outer portions 416a, 416b. The radially inwardly extending portions 418a, 418b may extend towards a center of the first portion 402 of the handle 400. In some cases, the radially inwardly extending portions 418a, 418b may contact or meet in the middle. However, this is not required. In some cases, free ends of the radially inwardly extending portions 418a, 418b may be spaced from one another. The radially inwardly extending portions 418a, 418b may extend into a slot 430 of the second portion 404 of the handle 400. At least a region of the radially inwardly extending portions 418a, 418b may include a lumen or through hole 422a, 422b. Regions of the radially inwardly extending portions 418a, 418b free from the lumens 422a, 422b may be substantially solid. The lumens 422a, 422b may extend generally orthogonal to the longitudinal axis of the first portion 402 of the handle 400. The lumens 422a, 422b may be aligned and configured to define a lumen extending across a height of the first portion 402 of the handle 400. The lumens 422a, 422b may be configured to receive a locking member 424 therethrough, as will be described in more detail herein. In some cases, the lumens 422a, 422b may be positioned within the intermediate region 410 of the first portion 402 of the handle 400. However, this is not required.

One or more gaps or openings 420a, 420b may be formed between the first and second body members 414a, 414b when they are assembled. These gaps 420a, 420b may extend from a proximal end to a distal end of the first and second body members 414a, 414b. The gaps 420a, 420b may be configured to receive the rails 436a, 436b of the second portion 404 to allow the first portion 402 to be axially displaced relative to the second portion 404. While not explicitly shown, in some configurations, the first portion 402 may further include an auxiliary lumen extending generally orthogonal to a longitudinal axis of the first portion 402. When so provided, the auxiliary lumen may extend from a laterally outward end to a laterally inward end and may be configured to receive an electrical connection member therethrough.

While not explicitly shown, a distal end region of the radially inwardly extending portions 418a, 418b may each include a recess extending proximally from the distal end of the first portion 402 of the handle 400. Collectively, the recesses may form a lumen (similar to the lumen 326 described herein) for receiving a proximal end of a wire filament (e.g., similar in form and function to wire filament 130) therein. The lumen may include a helical or bent region configured to facilitate retention of the wire filament within the lumen. Additionally, or alternatively, the wire filament may be secured using means such as, but not limited to, adhesives, friction fits, snap fits, press-fits, or the like. As the proximal end region of the wire filament is secured to the first portion 402 of the handle 400 via the lumen, axial movement of the first portion 402 may be translated to the wire filament.

The second portion 404 of the handle 400 may include a generally cylindrical body portion 426 including a gripping region 428 extending from a proximal end thereof. The gripping region 428 may be configured to provide a region for the clinician to hold the second portion 404 relative to the first portion 402 of the handle 400. The body portion 426 may include a slot 430 extending through a thickness or across a diameter thereof. The slot 430 may extend from a proximal end 432 to a distal end 434. On either side of the slot 430, the second portion of the handle 400 may include a first rail 436a and a second rail 436b. The first portion 402 of the handle 400 may be axially displaceable along the rails 436a, 436b with the radially inwardly extending portions 418a, 418b of the first portion 402 of the handle 400 axially displaceable within the slot 430.

The proximal end 432 of the slot 430 may be configured to create a mechanical stop when a proximal end of the radially inwardly extending portions 418a, 418b contacts the proximal end 432 of the slot 430 to limit proximal movement of the first portion 402 of the handle 400. Similarly, the distal end 434 of the slot 430 may be configured to create a mechanical stop when a distal end of the radially inwardly extending portions 418a, 418b contacts the distal end 434 of the slot 430 to limit distal movement of the first portion 402 of the handle 400. It is contemplated that the length of the slot 430 may be selected based on the desired proximal and/or distal movement of the first portion 402 (and the desired amount of bend at the distal tip).

The first portion 402 of the handle 400 may be assembled with the second portion 404 such that the body portion 426 (e.g., rails 436a, 436b) of the second portion 404 is disposed within the openings 420a, 420b formed by the first and second body members 414a, 414b of the first portion 402. The first portion 402 may be axially displaceable along an outer surface of the second portion 404.

The locking member 424 may extend through the lumens 422a, 422b of the radially inwardly extending portions 418a, 418b of the first portion 402 of the handle 400 and through the slot 430 of the second portion 404 of the handle 400. The locking member 424 may include a first control member 438a and a second control member 438b each configured to be positioned exterior to outer surface of the first portion 402 of the handle 400. The control members 438a, 438b may be knobs, levers, or other structures configured to allow the user to rotate the locking member 424 within the lumens 422a, 422b and the slot 430. In some cases, the control members 438a, 438b may include grooves 440a, 440b or other features, such as, but not limited to, bumps, divots, texturing, or the like to increase the gripability of the control members 438a, 438b. A stem 442 may extend between the first control member 438a and the second control member 438b. The stem 442 may have a knob or protruding region 444 extending radially from the outer surface of the stem 442. The knob 444 may be generally centrally located between the first and second control members 438a, 438b. However, this is not required. The knob 444 may have a generally oval or rectangular shape such that the knob 444 has a first width 446 when the first and second control members 438a, 438b are in an unlocked configuration and a second width 448 when the first and second control members 438a, 438b are in a locked configuration. The second width 448 may be greater than the first width 446. The first and second control members 438a, 438b may include visual indicia configured to identify the widest portion of the knob 444. For example, the first and second control members 438a, 438b may include color markings (such as, but not limited to, red markings) that align with (extend parallel to) the widest portion of the knob 444. When the color marking extends across the slot 430 (or generally orthogonal to a longitudinal axis thereof), the knob 444 is positioned such that the widest portion (having the second width 448) extends across the slot 430. In some cases, the first and second control members 438a, 438b may include color markings (such as, but not limited to, green markings) that align with (extend parallel to) the narrowest portion of the knob 444.

The first width 446 of the knob 444 (e.g., the narrowest portion thereof) may be less than a width 450 of the slot 430. When the knob 444 is positioned such that the first width 446 extends across the width 450 of the slot 430 (in a first, unlocked, configuration), the knob 444 may be spaced from the radially inward surface of the rails 436a, 436b such that the first portion 402 of the handle 400 moves freely along the second portion 404 to allow for the distal tip of the elongate tube to be bent into the desired configuration. The second width 448 of the knob 444 (e.g., the widest portion thereof) may be greater than a width 450 of the slot 430. When the distal tip of the elongate tube is in the desired configuration, the first and/or second control member 438a, 438b may be rotated to rotate the knob 444. The first or second control member 438a, 438b may be rotated until the second width 448 extends across the width 450 of the slot 430 and into a second, locked configuration. The visual indicia may be used to determine when the knob 444 is the in second, locked configuration. Since the second width 448 is greater than the width 450 of the slot 430, in the second, locked configuration, the knob 444 may exert a radially outward force on the radially inward surface of rails 436a, 436b of the second portion 404 of the handle 400. The outer surface of the rails 436a, 436b may frictionally engage the inner surface of the arcuate portions 416a, 416b of the first and second body members 414a, 414b. The frictional force between the rails 436a, 436b and the first and second body members 414a, 414b may prevent the axial movement of the first portion 402 of the handle 400 relative to the second portion 404 of the handle 400. To release the distal tip, the first or second control member 438a, 438b may be rotated until the first width 446 extends across the width 450 of the slot 430 and into the first, unlocked configuration as indicated by the visual indicia. In some cases, haptic feedback may be used to guide the positioning of the locking member 424.

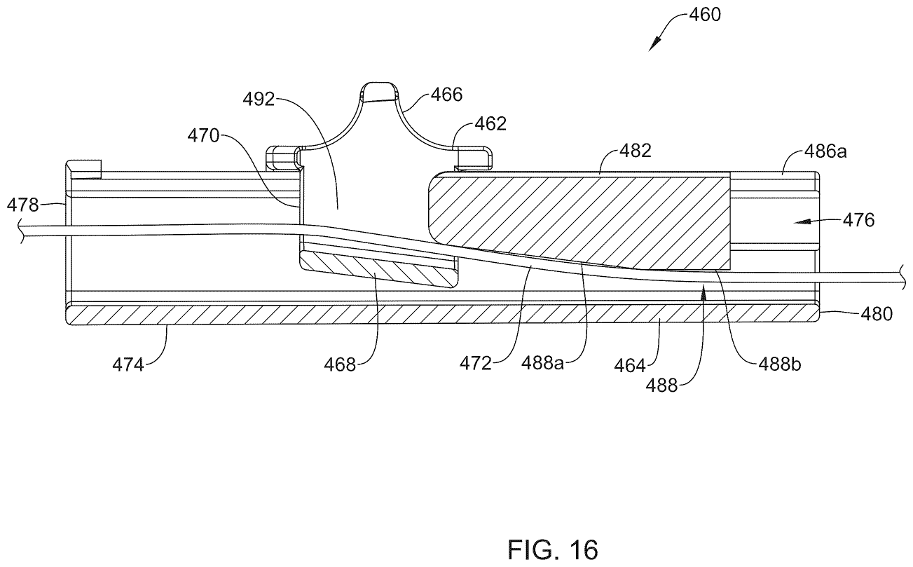

FIG. 15 is a perspective view of another illustrative locking mechanism 460 that may be used in place of the locking mechanism 152 of FIGS. 4 and 5. FIG. 16 is a cross-sectional view of the illustrative locking mechanism 460 of FIG. 15, taken at line 16-16 of FIG. 15. The locking mechanism 460 may be coupled with a distal end of the second portion of the handle using known techniques, such as, but not limited to, press-fits, friction fits, over-molding, heat-shrinking, or the like. The locking mechanism 460 may include a first portion 462 and a second portion 464. The first portion 462 of the locking mechanism 460 may be axially displaceable relative to the second portion 464 of the locking mechanism 460. For example, the first portion 462 may be proximally retracted relative to the second portion 464 to allow for free movement of the wire filament 472 and the first portion 462 may be distally advanced relative to the second portion 464 to prevent axial movement of the wire filament 472.

The first portion 462 may be a thumb or finger switch having an actuation region 466 and a coupling region 468 interconnected by a body region 470. A wire filament 472 may be fixedly coupled to the first portion of the handle (not explicitly shown) and extend through a channel 492 of the first portion 462 of the locking mechanism 460. The wire filament 472 may be similar in form and function to the wire filament 130 described herein. For example, actuation of the wire filament 472 at the handle may cause deflection of the distal tip of the accessory device.

The second portion 464 may have a generally “U”-shaped body member 474 having a first leg 486a, a second leg 486b laterally spaced from the first leg 486a and an interconnecting leg 486c extending therebetween. Collectively, the legs 486a, 486b, 486c may define an interior channel 476 extending from a proximal end 478 to a distal end 480 of the second portion 464. A retention wall 482 may extend laterally inwards into the channel 476. The retention wall 482 may be spaced from a first leg 486a to define a slot 484. A lower surface 488 of the retention wall 482 (e.g., a surface oriented towards the interconnecting leg 486c) may have a tapered or angled proximal end region 488a (e.g., extending generally non-parallel to the longitudinal axis of the second portion 464) and a distal end region 488b extending generally parallel to the longitudinal axis of the second portion 464.

A slot 490 may extend through the actuation region 466 and the body region 470 of the first portion 462 to allow the first portion 462 to move relative to the second portion 464. For example, at least some of the body region 470 may be positioned with the slot 484 of the second portion 464 of the locking mechanism 460 and at least some of the body region 470 may be positioned between the retention wall 482 and the second leg 486b. In some cases, one or more of the slots 484, 490, or other openings in the first or second portions 462, 464 may be enclosed or omitted to reduce the likelihood the wire filament 472 may radially exit the locking mechanism 460 unintentionally. The first portion 462 may be axially displaceable along the second portion 464 to selectively lock the wire filament 472 in a desired axial configuration. For example, in FIGS. 15 and 16, the first portion 462 may be in a first, or unlocked configuration. In this configuration, the wire filament 472 may be moved axially within the locking mechanism 460 to change a configuration of the distal tip. When the distal tip is in the desired configuration, the first portion 462 of the locking mechanism 460 may be distally advanced. As the first portion 462 is distally advanced to a second, locked configuration, the angled surface of the proximal end region 488a of the retention wall 482 may frictionally engage the wire filament 472 to prevent axial movement of the wire filament 472 and to maintain the distal tip in the desired configuration. To release the distal tip, the first portion 462 may be proximally retracted relative to the second portion 464.

In some cases, the locking mechanism 460 may be disposed between the distal end of the second portion of the handle and the proximal end of the elongate tube. In other examples, the locking mechanism 460 may be positioned at other locations, as desired. In some cases, the locking mechanism 460 may be formed with or incorporated into the first portion of the handle. In yet other examples, the locking mechanism 460 may be formed with or incorporated into the second portion of the handle.

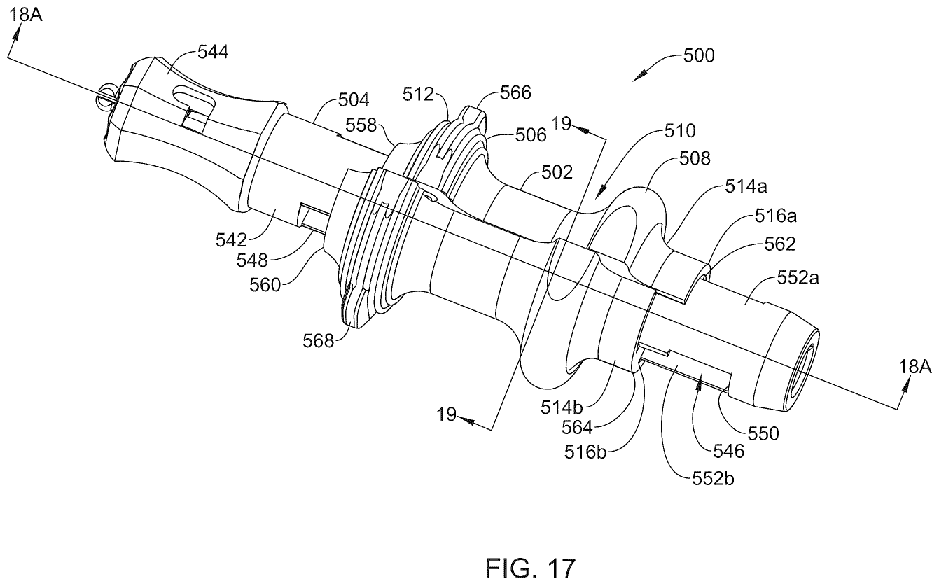

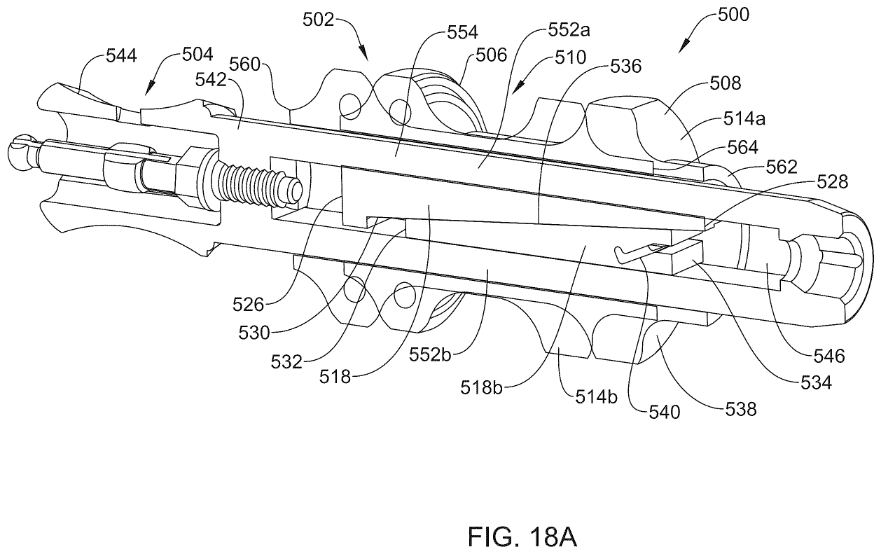

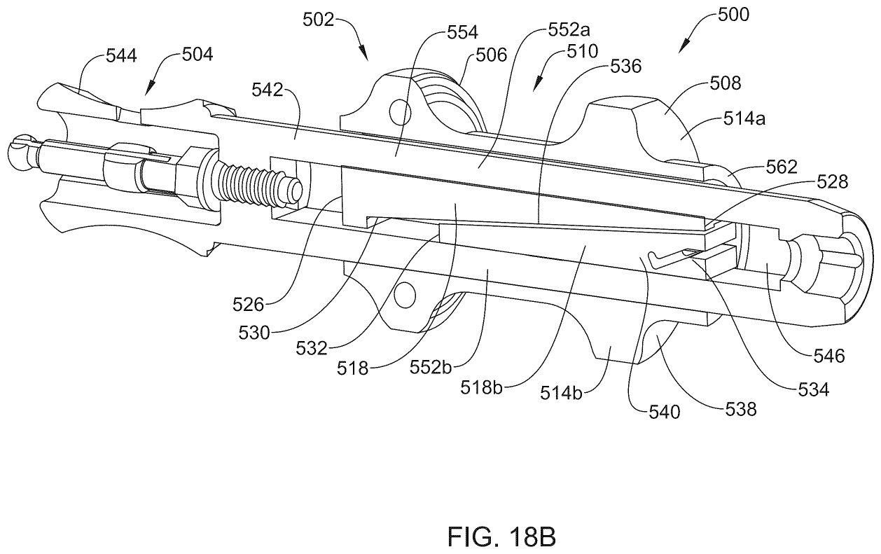

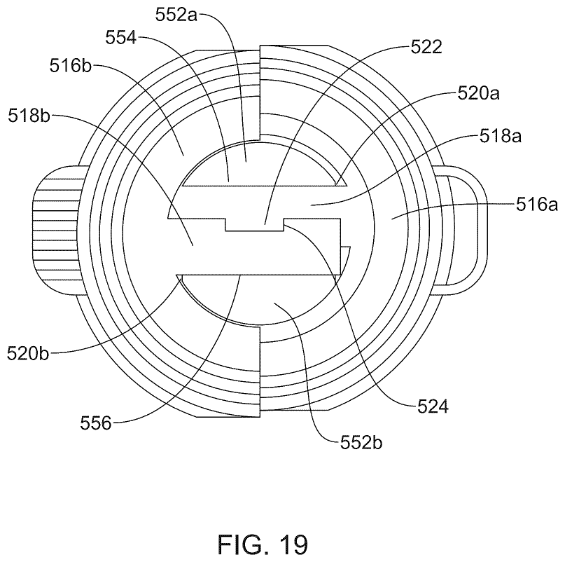

FIG. 17 is a perspective view of another illustrative handle 500, in a second, or locked configuration, that may be used to actuate and mechanically lock a wire filament of an accessory device. FIG. 18A is a cross-sectional view of the illustrative handle 500, taken at line 18A-18A of FIG. 17. FIG. 18B is a cross-sectional view of the illustrative handle 500 of FIG. 17 in a first, or unlocked, configuration. FIG. 19 is a cross-sectional view of the handle 500, taken at line 19-19 of FIG. 17. The handle 500 may be configured to be coupled to a proximal end of the elongate tube (not explicitly shown) of an accessory device (e.g., similar in form and function to the elongate tube 26 and accessory device 14 described herein). The handle 500 may include a first portion 502 and a second portion 504. The first portion 502 of the handle 500 may be axially displaceable relative to the second portion 504 of the handle 500. For example, the first portion 502 may be proximally retracted relative to the second portion 504 to deflect the distal tip of the elongate tube in a first direction and/or the first portion 502 may be distally advanced relative to the second portion 504 to deflect the distal tip in a second direction opposite the first direction.

The first portion 502 may include a first, or proximal, gripping region 506 and a second, or distal, gripping region 508. The first and second gripping regions 506, 508 may have an enlarged cross-sectional profile relative to an intermediate region 510 of the first portion 502. This may provide a region for the user to push or pull against to move the first portion 502 relative to the second portion 504. In some cases, one or both of the gripping regions 506, 508 may include texturing 512, such as raised ridges, grooves, bumps, or the like to increase the gripability of the first portion 502.

The first portion 502 may be formed from a first body member 514a and a second body member 514b. Generally, the first or second body member 514a, 514b may be axially displaced relative to the other of the first or second body member 514a, 514b to move the first portion 502 between an unlocked configuration (FIG. 18B) where axial movement of the first portion 502 and the wire filament relative to the second portion 504 is permitted and a locked configuration (FIGS. 17 and 18A) where axially movement of the first portion 502 and the wire filament relative to the second portion 504 is precluded. The first and second body members 514a, 514b may be moved together or as unit to deflect the distal tip of the elongate shaft.

Each body member 514a, 514b may include an arcuate outer portion 516a, 516b and a radially inward extending portion 518a, 518b. A wall thickness of the arcuate outer portion 516a, 516b may vary along a length thereof. Each of the arcuate outer portions 516a, 516b may have an arc length of approximately 180° such that collectively, the arcuate outer portions 516a, 516b form a complete or substantially complete ring. In some cases, a small gap may be present between adjacent surfaces of the first and second arcuate outer portions 516a, 516b. The first and second body members 514a, 514b may be fixedly or releasably coupled to one another over the second portion 504 of the handle 500. In some cases, the first and second body members 514a, 514b may be secured using snap fits, such as a mating tab 522 (e.g., a protrusion) and mating recess 524 (e.g., a groove), friction fits, press-fits, or the like. In some cases, the mating tab 522 may be formed in the radially inward extending portion 518a of the first body member 514a and the mating recess 524 may be formed in the radially inward extending portion 518b of the second body member 514b. However, this is not required. When so provided, the coupling mechanisms may be provided in other portions of the first or second body members 514a, 514b, as desired.

The radially inwardly extending portions 518a, 518b may extend towards a center of the first portion 502 of the handle 500 from opposing sides of thereof. In some cases, the radially inwardly extending portions 518a, 518b may contact or meet. However, this is not required. The radially inwardly extending portions 518a, 518b may extend into a slot 546 of the second portion 504 of the handle 500. One or more gaps or openings 520a, 520b may be formed between the first and second body members 514a, 514b when they are assembled. These openings 520a, 520b may extend from a proximal end to a distal end of the first and second body members 514a, 514b. The openings 520a, 520b may be configured to receive the rails 552a, 552b of the second portion 504 to allow the first portion 502 to be axially displaced relative to the second portion 504. While not explicitly shown, in some configurations, the first portion 502 may further include an auxiliary lumen extending generally orthogonal to a longitudinal axis of the first portion 502. When so provided, the auxiliary lumen may extend from a laterally outward end to a laterally inward end and may be configured to receive an electrical connection member therethrough.

The first radially inward extending portion 518a may extend from a first, or proximal, end 526 to a second, or distal, end 528. The first radially inwardly extending portion 518a may include a proximal ledge configured to extend substantially across a width of the slot 546 of the second portion 504. Together with a proximal end 532 of the second radially inward extending portion 518b, the proximal ledge may form a mechanical stop to limit proximal movement of the second body member 514b relative to the first body member 514a. The first radially inward extending portion 518a may have a thickness that tapers or reduces in the distal direction from a distal end of the proximal ledge to the distal end 528 of the first radially inward extending portion 518a to define a first sloped surface 530.

The second radially inward extending portion 518b may extend from a first, or proximal, end 532 to a second, or distal, end 534. The second radially inward extending portion 518b may have a thickness that tapers or reduces in the proximal direction from the distal end 534 to the proximal end 532 of the second radially inward extending portion 518b to define a second sloped surface 536. The first and second sloped surfaces 530, 536 may extend parallel to one another. In the first, unlocked configuration illustrated in FIG. 18B, the first and second sloped surfaces 530, 536 may be spaced from one another. In the second, locked configuration illustrated in FIG. 18A, the first and second sloped surfaces 530, 536 may contact or frictionally engage one another.

The second radially inward extending portion 518b may further include a lumen 538 extending proximally from the distal end 534 thereof. The lumen 538 may be configured to receive a proximal end of a wire filament therein (e.g., similar in form and function to wire filament 130). The lumen 538 may include a helical or angled region 540 configured to facilitate retention of the wire filament within the lumen 538. Additionally, or alternatively, the wire filament may be secured using means such as, but not limited to, adhesives, friction fits, snap fits, press-fits, or the like. As the proximal end region of the wire filament is secured to the first portion 502 of the handle 500 via the lumen 538, axial movement of the first portion 502 may be translated to the wire filament.

The second portion 504 of the handle 500 may include a generally cylindrical body portion 542 including a gripping region 544 extending from a proximal end thereof. The gripping region 544 may be configured to provide a region for the clinician to hold the second portion 504 relative to the first portion 502 of the handle 500. The body portion 542 may include a slot 546 extending through a thickness or across a diameter thereof. The slot 546 may extend from a proximal end 548 to a distal end 550. On either side of the slot 546, the second portion of the handle 500 may include a first rail 552a and a second rail 552b. The first portion 502 of the handle 500 may be axially displaceable along the rails 552a, 552b with the radially inwardly extending portions 518a, 518b of the first portion 502 of the handle 500 axially displaceable within the slot 546.

The proximal end 548 of the slot 546 may be configured to create a mechanical stop when a proximal end of the radially inwardly extending portions 518a, 518b contacts the proximal end 548 of the slot 546 to limit proximal movement of the first portion 502 of the handle 500. Similarly, the distal end 550 of the slot 546 may be configured to create a mechanical stop when a distal end of the radially inwardly extending portions 518a, 518b contacts the distal end 550 of the slot 546 to limit distal movement of the first portion 502 of the handle 500. It is contemplated that the length of the slot 546 may be selected based on the desired proximal and/or distal movement of the first portion 502 (and the desired amount of bend at the distal tip).

The first portion 502 of the handle 500 may be assembled with the second portion 504 such that the body portion 542 (e.g., rails 552a, 552b) of the second portion 504 is disposed within the openings 520a, 520b formed by the first and second body members 514a, 514b of the first portion 502. The first portion 502 may be axially displaceable along an outer surface of the second portion 504.

When movement or deflection of the distal tip of the elongate tube is desired, the user may align a proximal end 558 of the first body member 514a with a proximal end 560 of the second body member 514b and/or a distal end 562 of the first body member 514a with a distal end 562 of the second body member 514b. In this configuration, the first and second sloped surfaces 530, 536 may be spaced from one another. The user may then proximally retract or distally advance both the first and second body members 514a, 514b together to axially displace the wire filament to achieve the desired configuration of the distal tip. When the distal tip is in the desired configuration, the first body member 514a may be distally advanced while the second body member 514b is held stationary. Alternatively, the second body member 514b may be proximally retracted while the first body member 514a is held stationary. In yet another example, the first body member 514a may be distally advanced while the second body member 514b is proximally retracted. It is contemplated that the direction of movement of the two body members 514a, 514b relative to the other may be determined by a direction of slope of the sloped surfaces 530, 536. When the first or second body member 514a, 514b is axially offset from the other (e.g., in opposite directions), the wedge shape of the first and second sloped surfaces 530, 536 push against one another causing a laterally outward surface 554 of the first radially inwardly extending portion 518a to push against a surface of the first rail 552a and a laterally outward surface 556 of the second radially inwardly extending portion 518b to push against a surface of the second rail 552b. The frictional force between the rails 552a, 552b and the first and second radially inwardly extending portions 518a, 518b may prevent the axial movement of the first portion 502 of the handle 500 relative to the second portion 504 of the handle 500 to lock the distal tip in the desired configuration. To release the distal tip, the first and/or second body member 514a, 514b may be axially displaced to align the proximal end 558 of the first body member 514a with a proximal end 560 of the second body member 514b and/or a distal end 562 of the first body member 514a with a distal end 562 of the second body member 514b which may disengage the frictional engagement between the rails 552a, 552b and the first and second radially inwardly extending portions 518a, 518b to allow for axial displacement of the first portion 502 of the handle 500 relative to the second portion 504 of the handle 500.

In some configurations, the first and second body members 514a, 514b may include laterally extending members 566, 568. The laterally extending members 566, 568 may provide a gripping region to facilitate axial moment of the first and second body members 514a, 514b relative to each other. While the illustrated embodiment depicts the laterally extending members 566, 568 as extending from the proximal gripping region 506, the laterally extending members 566, 568 may be positioned anywhere along a length of the first or second body members 514a, 514b. Further, more than two or fewer than two laterally extending members 566, 568 may be provided.

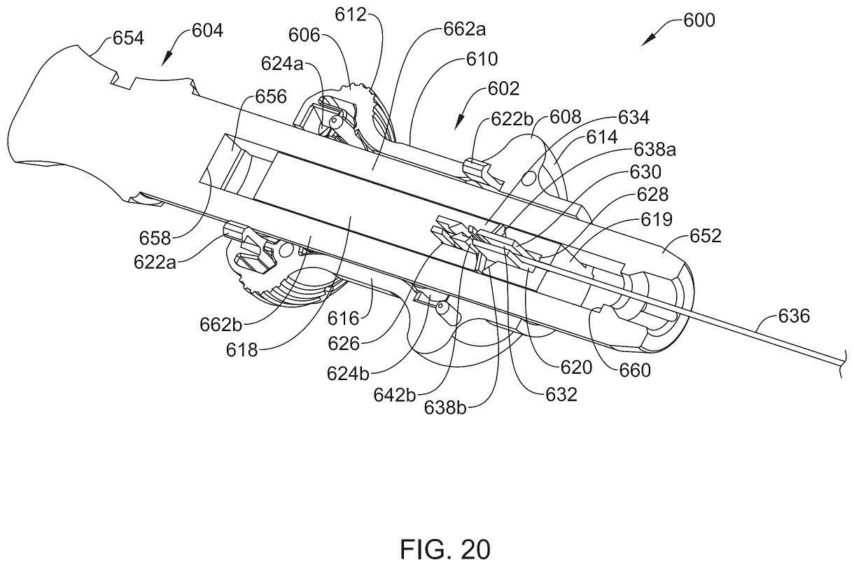

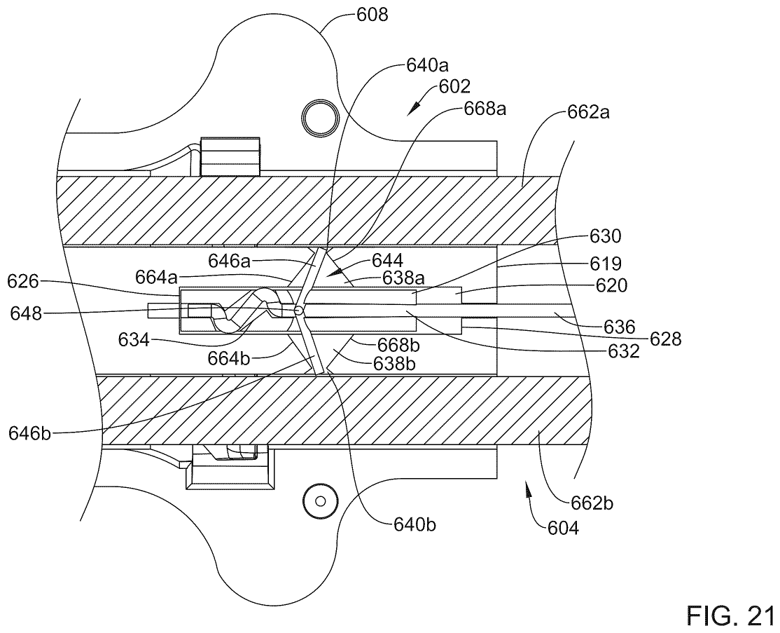

FIG. 20 is a perspective view of a partial cross-section of another illustrative handle 600 that may be used to actuate and mechanically lock a wire filament of an accessory device. FIG. 21 is a top view of a portion of the illustrative handle 600. The handle 600 may be configured to be coupled to a proximal end of the elongate tube (not explicitly shown) of an accessory device (e.g., similar in form and function to the elongate tube 26 and accessory device 14 described herein). The handle 600 may include a first portion 602 and a second portion 604. The first portion 602 of the handle 600 may be axially displaceable relative to the second portion 604 of the handle 600. For example, the first portion 602 may be proximally retracted relative to the second portion 604 to deflect the distal tip of the elongate tube in a first direction and/or the first portion 602 may be distally advanced relative to the second portion 604 to deflect the distal tip in a second direction opposite the first direction.

The first portion 602 may include a first, or proximal, gripping region 606 and a second, or distal, gripping region 608. The first and second gripping regions 606, 608 may have an enlarged cross-sectional profile relative to an intermediate region 610 of the first portion 602. This may provide a region for the user to push or pull against to move the first portion 602 relative to the second portion 604. In some cases, one or both of the gripping regions 606, 608 may include texturing 612, such as raised ridges, grooves, bumps, or the like to increase the gripability of the first portion 602.

The first portion 602 may be formed from a first body member 614 and a second body member (not explicitly shown). The second body member may have a same structure as the first body member 614. The first body member 614 may include an arcuate outer portion 616 and a radially inward extending portion 618. A wall thickness of the arcuate outer portion 616 may vary along a length thereof. The arcuate outer portion 616 may have an arc length of approximately 180° such that collectively (e.g., when the first body member 614 and the second body member are assembled), the arcuate outer portions 616 form a complete or substantially complete ring (for example, similar to handles 300, 400 described herein). In some cases, a small gap may be present between adjacent surfaces of the first and second arcuate outer portions 616. The first and second body members 614 may be fixedly or releasably coupled to one another over the second portion 604 of the handle 600. In some cases, the first and second body members 614 may be secured using mating latches 622a, 622b and slots, tabs, or ridges 624a, 624b, snap fits, friction fits, press-fits, or the like. For example, latches 622a, 622b on the first body member 614 may be configured to engage mating slots, tabs or ridges (not explicitly shown) on the second body member and latches (not explicitly shown) on the second body member may be configured to engage mating slots, tabs or ridges 624a, 624b on the first body member 614. In some cases, the coupling mechanisms 622a, 622b, 624a, 624b may be adjacent to the gripping regions 606, 608. However, this is not required. When so provided, the coupling mechanisms may be provided in other portions of the first or second body members 614, as desired.

The radially inwardly extending portion 618 may extend towards a center of the first portion 602 of the handle 600. In some cases, the radially inwardly extending portions 618 of the first and second body members 614 may contact or meet. However, this is not required. The radially inwardly extending portion 618 may extend into a slot 656 of the second portion 604 of the handle 600. One or more gaps or openings (not explicitly shown) may be formed between the first and second body members 614 when they are assembled. These gaps may extend from a proximal end to a distal end of the first and second body members 614. The gaps may be configured to receive the rails 662a, 662b of the second portion 604 to allow the first portion 602 to be axially displaced relative to the second portion 604. While not explicitly shown, in some configurations, the first portion 602 may further include an auxiliary lumen extending generally orthogonal to a longitudinal axis of the first portion 602. When so provided, the auxiliary lumen may extend from a laterally outward end to a laterally inward end and may be configured to receive an electrical connection member therethrough.

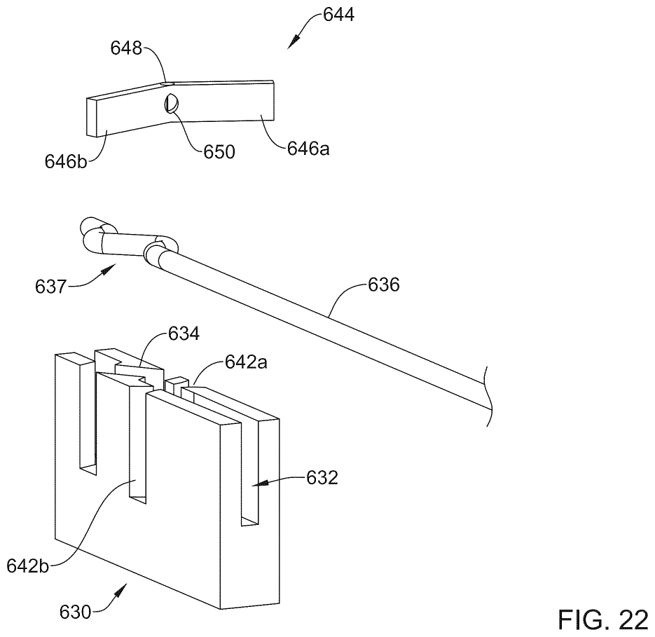

The radially inward extending portion 618 may include a recess 620 extending from a proximal end 626 to a distal end 628. The distal end 628 of the recess 620 may be proximal to a distal end 619 of the radially inward extending portion 618. The recess 620 may have a depth that extends through less than an entire thickness of the radially inward extending portion 618. The recess 620 may be configured to slidably receive a shuttle 630 therein. The shuttle 630 may have an axial length that is less than an axial length of the recess 620. Generally, the shuttle 630 may move proximally or distally within the recess 620 as the first portion 602 of the handle 600 is actuated to selectively engage and disengage a spring member 644 locking mechanism. Referring additionally to FIG. 22, which is an exploded perspective view of the illustrative shuttle 630, wire filament 636, and spring member 644, the shuttle 630 may include a generally axially extending channel 632 extending from a proximal end to a distal end thereof. The channel 632 may be configured to receive a proximal end region of a wire filament 636. In some cases, the channel 632 may include an angled or zig zag region 634 configured to receive a bent or helical portion 637 of the wire filament 636 and facilitate retention of the wire filament 636 within the channel 632. Additionally, or alternatively, the wire filament 636 may be secured using means such as, but not limited to, adhesives, friction fits, snap fits, press-fits, or the like. The shuttle 630 may further include one or more laterally extending slots 642a, 642b configured to allow the arms 646a, 646b of the spring member 644 to extend laterally beyond the lateral sides of the shuttle 630.

The recess 620 may have a generally rectangular cross-section extending from the proximal end 626 to the distal end 628 thereof. An intermediate region of the recess 620 may have a first lateral section 638a having a generally triangular or hourglass cross-section extending from a first lateral side of the recess 620 and a second lateral section 638b having a generally triangular or hourglass cross-section extending from a second lateral side of the recess 620. The first and second lateral sections 638a, 638b may extend to opposing lateral edges of the radially inward extending portion 618 to define lateral openings 640a, 640b in the radially inward extending portion 618. An axial dimension (e.g., proximal to distal) of the first lateral section 638a may reduce in the laterally outward direction to define a proximal tapered surface 664a and a distal tapered surface 668a. Similarly, an axial dimension (e.g., proximal to distal) of the second lateral section 638b may reduce in the laterally outward direction to define a proximal tapered surface 664b and a distal tapered surface 668b. The first and second lateral sections 638a, 638b may be configured to receive the arms 646a, 646b of a spring member 644. In some configurations, the arms 646a, 646b may extend through the lateral openings 640a, 640b.

The spring member 644 may include a first laterally extending arm 646a and a second laterally extending arm 646b. The arms 646a, 646b may be hingedly or pivotable coupled to one another at a hinge point 648. In some cases, the arms 646a, 646b may be separate components mechanically coupled with, for example, a pin. In other examples, the arms 646a, 646b may be formed as flexible single monolithic piece. A central aperture 650 may extend through a thickness of the spring member 644. The central aperture 650 may be configured to receive a portion of the wire filament 636 therethrough. When assembled with the shuttle 630 and the wire filament 636, the spring member 644 may be positioned distal to the bent portion 637 of the wire filament 636.

The spring member 644 may be formed from a resilient or shape memory material configured to be temporarily deformed under the application of an applied force (e.g., proximal or distal movement of the first portion 602 of the handle 600) and return to the original unbiased configuration in the absence of the applied force. The original unbiased configuration of the spring member 644 positions the arms 646a, 646b through the lateral openings 640a, 640b and into frictional engagement with the second portion 604 of the handle 600. The biased configuration of the spring member 644 may reduce a lateral width of the spring member 644 to space the arms 646a, 646b from the second portion 604 of the handle 600.

The second portion 604 of the handle 600 may include a generally cylindrical body portion 652 including a gripping region 654 extending from a proximal end thereof. The gripping region 654 may be configured to provide a region for the clinician to hold the second portion 604 relative to the first portion 602 of the handle 600. The body portion 652 may include a slot 656 extending through a thickness or across a diameter thereof. The slot 656 may extend from a proximal end 658 to a distal end 660. On either side of the slot 656, the second portion of the handle 600 may include a first rail 662a and a second rail 662b. The first portion 602 of the handle 600 may be axially displaceable along the rails 662a, 662b with the radially inwardly extending portions 618 of the first portion 602 of the handle 600 axially displaceable within the slot 656.

The proximal end 658 of the slot 656 may be configured to create a mechanical stop when a proximal end of the radially inwardly extending portion 618 contacts the proximal end 658 of the slot 656 to limit proximal movement of the first portion 602 of the handle 600. Similarly, the distal end 660 of the slot 656 may be configured to create a mechanical stop when a distal end of the radially inwardly extending portion 618 contacts the distal end 660 of the slot 656 to limit distal movement of the first portion 602 of the handle 600. It is contemplated that the length of the slot 656 may be selected based on the desired proximal and/or distal movement of the first portion 602 (and the desired amount of bend at the distal tip).

The first portion 602 of the handle 600 may be assembled with the second portion 604 such that the body portion 652 (e.g., rails 662a, 662b) of the second portion 604 is disposed within the openings formed by the first and second body members 614 of the first portion 602. The first portion 602 may be axially displaceable along an outer surface of the second portion 604.

When movement or deflection of the distal tip of the elongate tube is desired, the user may distally advance or proximally retract the first portion 602 of the handle 600 relative to the second portion 604 of the handle 600. As the first portion 602 is moved relative to the second portion 604, the spring member 644 may hinge, flex, or pivot from a first laterally expanded configuration to a second laterally collapsed configuration. When the spring member 644 is in the first laterally expanded configuration, the lateral edges of the arms 646a, 646b may frictionally engage the rails 662a, 662b of the second portion 604 of the handle 600 to retain the first portion 602 in a fixed configuration relative to the second portion 604 without requiring the user to hold the first portion 602.

As a user proximally retracts the first portion 602 of the handle 600, the shuttle 630 moves proximally within the recess 620 and the proximal sides of the arms 646a, 646b may engage the proximal tapered surfaces 664a, 664b of the first and second lateral sections 638a, 638b. As the arms 646a, 646b engage the surfaces 664a, 664b, the spring member 644 bows or flexes to reduce a lateral width thereof. In the bowed or flexed configuration, the free ends of the arms 646a, 646b are spaced from the rails 662a, 662b of the second portion 604 to allow for free movement of the first portion 602 of the handle 600. When proximal actuation of the first portion 602 of the handle 600 is stopped, the spring member 644 may return to its original unbiased configuration in which the arms 646a, 646b extend through the lateral openings 640a, 640b to frictionally engage the rails 662a, 662b of the second portion 604 of the handle 600.

Similarly, as a user distally advances the first portion 602 of the handle 600, the shuttle 630 moves distally within the recess 620 and the distal sides of the arms 646a, 646b may engage the distal tapered surfaces 668a, 668b of the first and second lateral sections 638a, 638b. As the arms 646a, 646b engage the surfaces 668a, 668b, the spring member 644 bows or flexes to reduce a lateral width thereof. In the bowed or flexed configuration, the free ends of the arms 646a, 646b are spaced from the rails 662a, 662b of the second portion 604 to allow for free movement of the first portion 602 of the handle 600. When distal actuation of the first portion 602 of the handle 600 is stopped, the spring member 644 may return to its original unbiased configuration in which the arms 646a, 646b extend through the lateral openings 640a, 640b to frictionally engage the rails 662a, 662b of the second portion 604 of the handle 600.

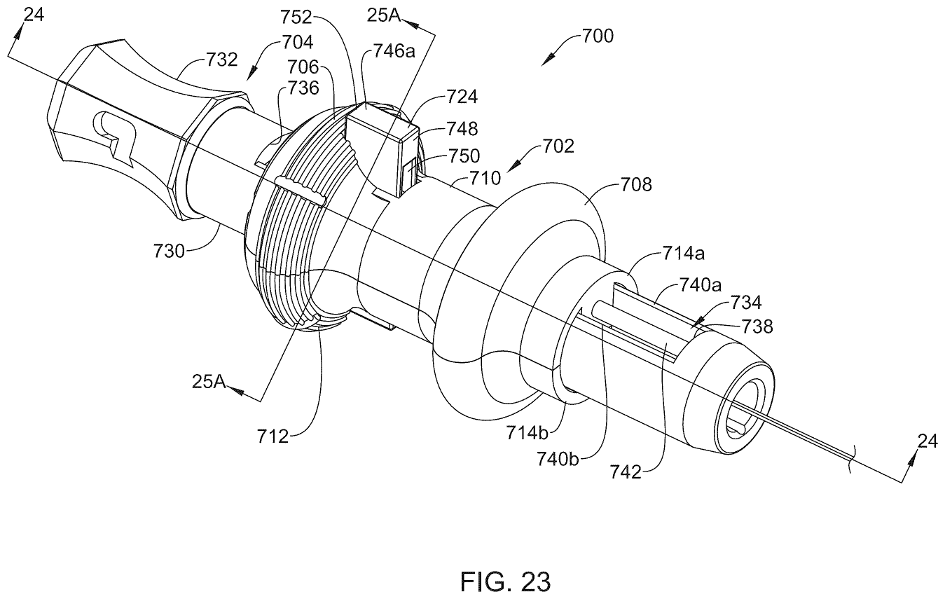

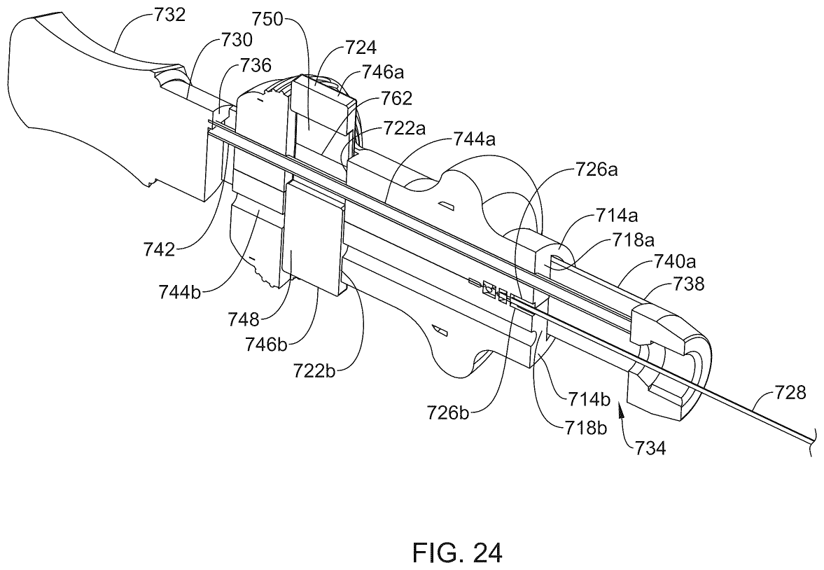

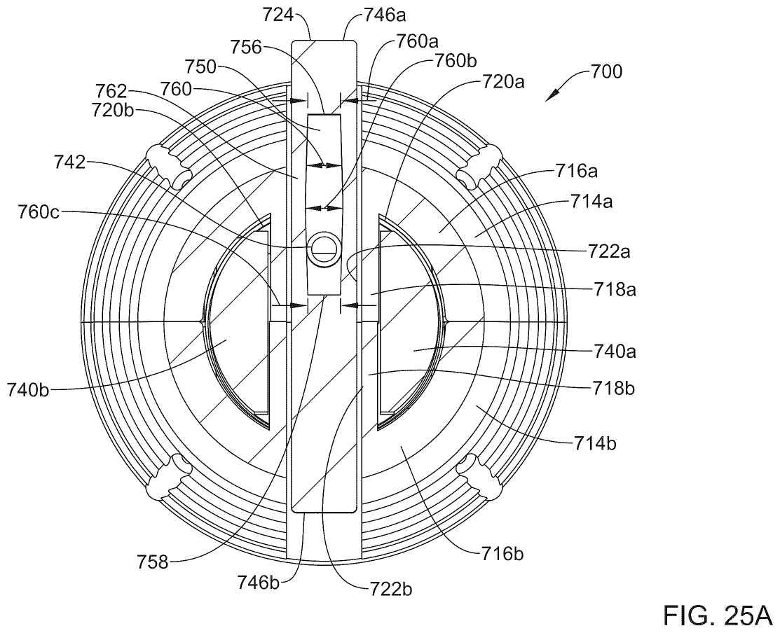

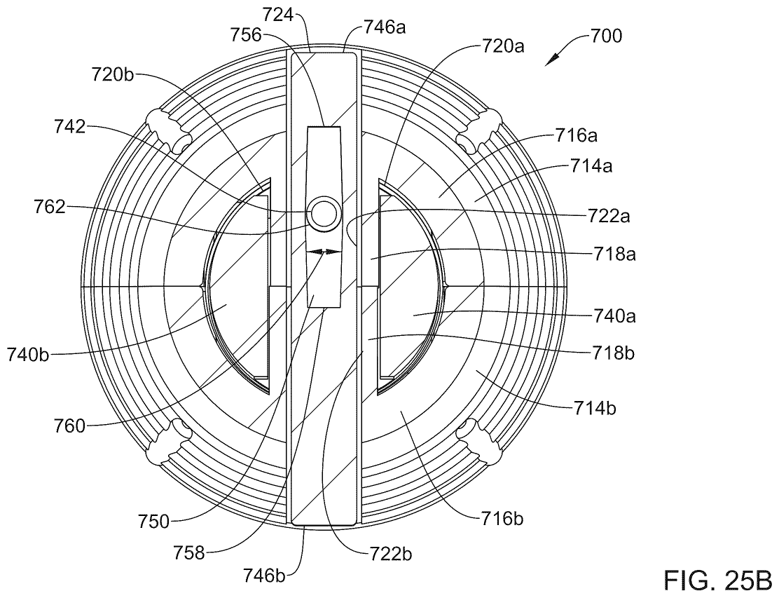

FIG. 23 is a schematic perspective view of another illustrative handle 700 that may be used to actuate and mechanically lock a wire filament of an accessory device. FIG. 24 is a cross-sectional view of the illustrative handle 700, taken at line 24-24 of FIG. 23. FIG. 25A is a cross-sectional view of the illustrative handle 700 of FIG. 23, taken at line 25A-25A with a locking member 724 in a second, locked, configuration. FIG. 25B is the cross-sectional view of FIG. 25A with the locking member 724 in a first, unlocked, configuration. The handle 700 may be configured to be coupled to a proximal end of the elongate tube (not explicitly shown) of an accessory device (e.g., similar in form and function to the elongate tube 26 and accessory device 14 described herein). The handle 700 may include a first portion 702 and a second portion 704. The first portion 702 of the handle 700 may be axially displaceable relative to the second portion 704 of the handle 700. For example, the first portion 702 may be proximally retracted relative to the second portion 704 to deflect the distal tip of the elongate tube in a first direction and the first portion 702 may be distally advanced relative to the second portion 704 to deflect the distal tip in a second direction opposite the first direction.

The first portion 702 may include a first, or proximal, gripping region 706 and a second, or distal, gripping region 708. The first and second gripping regions 706, 708 may have an enlarged cross-sectional profile relative to an intermediate region 710 of the first portion 702. This may provide a region for the user to push or pull against to move the first portion 702 relative to the second portion 704. In some cases, one or both of the gripping regions 706, 708 may include texturing 712, such as raised ridges, grooves, bumps, or the like to increase the gripability of the first portion 702.

The first portion 702 may be formed from a first body member 714a and a second body member 714b. The first and second body members 714a, 714b may be fixedly or releasably coupled to one another over the second portion 704 of the handle 700. In some cases, the first and second body members 714a, 714b may be secured using mating latches and tabs or ridges, snap fits, friction fits, press-fits, or the like. Each body member 714a, 714b may include an arcuate outer portion 716a, 716b and a radially inward extending portion 718a, 718b. A wall thickness of the arcuate outer portion 716a, 716b may vary along a length thereof. Each of the arcuate outer portions 716a, 716b may have an arc length of approximately 180° such that collectively, the arcuate outer portions 716a, 716b form a complete or substantially complete ring. In some cases, a small gap may be present between adjacent surfaces of the first and second arcuate outer portions 716a, 716b. The radially inwardly extending portions 718a, 718b may extend towards a center of the first portion 702 of the handle 700. In some cases, the radially inwardly extending portions 718a, 718b may contact or meet in the middle. However, this is not required. In some cases, free ends of the radially inwardly extending portions 718a, 718b may be spaced from one another. The radially inwardly extending portions 718a, 718b may extend into a slot 734 of the second portion 704 of the handle 700. At least a region of the radially inwardly extending portions 718a, 718b may include a slot or through hole 722a, 722b. Regions of the radially inwardly extending portions 718a, 718b free from the slots 722a, 722b may be substantially solid. The slots 722a, 722b may extend generally orthogonal to the longitudinal axis of the first portion 702 of the handle 700. The slots 722a, 722b may be aligned and configured to define a lumen extending across a height of the first portion 702 of the handle 700. The slots 722a, 722b may be configured to receive a locking member 724 therethrough, as will be described in more detail herein. In some cases, the slots 722a, 722b may be positioned within the intermediate region 710 of the first portion 702 of the handle 700. However, this is not required.

The radially inwardly extending portions 718a, 718b may further include an axially extending lumen 744a, 744b extending from a proximal end to a distal end of the first portion 702 of the handle 700. At least one of the axially extending lumens 744a, 744b may be configured to receive an elongate member 742 of the second portion 704 of the handle 700. It is contemplated that one of the first or second radially inwardly extending portions 718a, 718b may not include an axially extending lumen 744a, 744b.

One or more gaps or openings 720a, 720b may be formed between the first and second body members 714a, 714b when they are assembled. These gaps 720a, 720b may extend from a proximal end to a distal end of the first and second body members 714a, 714b. The gaps 720a, 720b may be configured to receive the rails 740a, 740b of the second portion 704 to allow the first portion 702 to be axially displaced relative to the second portion 704. While not explicitly shown, in some configurations, the first portion 702 may further include an auxiliary lumen extending generally orthogonal to a longitudinal axis of the first portion 702. When so provided, the auxiliary lumen may extend from a laterally outward end to a laterally inward end and may be configured to receive an electrical connection member therethrough.

A distal end region of the radially inwardly extending portions 718a, 718b may each include a recess 726a, 726b extending proximally from the distal end of the first portion 702 of the handle 700. Collectively, the recesses 726a, 726b may form a lumen (similar to the lumen 326 described herein) for receiving a proximal end of a wire filament 728 therein. The lumen may include a helical or angled region configured to facilitate retention of the wire filament 728 within the lumen. Additionally, or alternatively, the wire filament 728 may be secured using means such as, but not limited to, adhesives, friction fits, snap fits, press-fits, or the like. As the proximal end region of the wire filament 728 is secured to the first portion 702 of the handle 700 via the lumen, axial movement of the first portion 702 may be translated to the wire filament.

The second portion 704 of the handle 700 may include a generally cylindrical body portion 730 including a gripping region 732 extending from a proximal end thereof. The gripping region 732 may be configured to provide a region for the clinician to hold the second portion 704 relative to the first portion 702 of the handle 700. The body portion 730 may include a slot 734 extending through a thickness or across a diameter thereof. The slot 734 may extend from a proximal end 736 to a distal end 738. On either side of the slot 734, the second portion of the handle 700 may include a first rail 740a and a second rail 740b. The first portion 702 of the handle 700 may be axially displaceable along the rails 740a, 740b with the radially inwardly extending portions 718a, 718b of the first portion 702 of the handle 700 axially displaceable within the slot 734.

The second portion 704 of the handle 700 may further include an elongate member 742 such as a tube or solid shaft extending from the proximal end 736 to the distal end 738 thereof. In some configurations, the elongate member 742 may be formed from a flexible or deformable material. However, this is not required. The elongate member 742 may be radially offset from a central longitudinal axis of the second portion 704 of the handle 700.

The proximal end 736 of the slot 734 may be configured to create a mechanical stop when a proximal end of the radially inwardly extending portions 718a, 718b contacts the proximal end 736 of the slot 734 to limit proximal movement of the first portion 702 of the handle 700. Similarly, the distal end 738 of the slot 734 may be configured to create a mechanical stop when a distal end of the radially inwardly extending portions 718a, 718b contacts the distal end 738 of the slot 734 to limit distal movement of the first portion 702 of the handle 700. It is contemplated that the length of the slot 734 may be selected based on the desired proximal and/or distal movement of the first portion 702 (and the desired amount of bend at the distal tip).

The first portion 702 of the handle 700 may be assembled with the second portion 704 such that the body portion 730 (e.g., rails 740a, 740b) of the second portion 704 is disposed within the openings 720a, 720b formed by the first and second body members 714a, 714b of the first portion 702. The first portion 702 may be axially displaceable along an outer surface of the second portion 704.

The locking member 724 may extend through the slots 722a, 722b of the radially inward extending portions 718a, 718b of the first portion 702 of the handle 700 and through the slot 734 of the second portion 704 of the handle 700. The locking member 724 may extend from a first actuation member 746a and a second actuation member 746b each configured to be positioned exterior to outer surface of the first portion 702 of the handle 700. In some cases, the actuation members 746a, 746b may be radially outward surfaces of the locking member 724. Generally, the locking member 724 may be configured to be radially displaced to selectively lock the first portion 702 of the handle 700 relative to the second portion 704 of the handle 700. For example, the locking member 724 may be movable between a first unlocked configuration (FIG. 25B) where the first portion 702 of the handle 700 moves freely relative to the second portion 704 of the handle 700 and a second, locked configuration wherein the first portion 702 of the handle 700 is fixed relative to the second portion 704 of the handle 700.

The locking member 724 may include a body member 748 extending between the first actuation member 746a and the second actuation member 746b. The body member 748 may be generally solid with a channel 750 extending from a proximal end 752 to a distal end 754 of the locking member 724. The channel 750 may have a height extending from a first end 756 to a second end 758 thereof. The height of the channel 750 may be less than a height of the locking member 724. A width 760 of the channel 750 may vary from the first end 756 to the second end thereof. For example, the width 760 of the channel 750 may increase from a first width 760a adjacent to the first end 756 thereof to second width 760b at a center point 762 of the channel 750 (e.g., halfway between the first end 756 and the second end 758). The second width 760b may be greater than the first width 760a. The width 760 may transition from the first width 760a to the second width 760b in a gradual tapered manner. The width 760 of the channel 750 may decrease from the second width 760b at a center point 762 of the channel 750 to a third width 760c adjacent to the second end 758 thereof. The second width 760b may be greater than the third width 760c. In some cases, the first width 760a and the third width 760c may be the same or approximately the same. However, this is not required. The width 760 may transition from the second width 760b to the third width 760c in a gradual tapered manner. The channel 750 may have a generally truncated diamond-like cross-sectional shape.

The elongate member 742 of the second portion 704 of the handle 700 may extend through the channel 750 of the locking member 724. The first and third widths 760a, 760c may be less than a diameter of the elongate member 742 while the second width 760b may be greater than the diameter of the elongate member 742. When the locking member 724 is positioned such that the elongate member 742 is adjacent to the center point 762 of the channel 750 (e.g., adjacent to the second width 760b), the first portion 702 of the handle 700 may be proximally or distally displaceable along the second portion 704 of the handle 700. Said differently, when the locking member 724 is positioned such that the elongate member 742 is adjacent to the center point 762 of the channel 750, the locking member 724 is in the unlocked configuration. In the unlocked configuration, the first and second actuation members 746a, 746b may be radially aligned with an outer surface of the proximal gripping region 706. However, this is not required.

When the distal tip has been actuated to the desired configuration, the user may depress either the first or the second actuation member 746a, 746b to radially displace the locking member 724. In the illustrated configuration of FIG. 25A, the second actuation member 746b has been pushed, depressed, or otherwise actuated. As the locking member 724 is actuated, the channel 750 slides along the outer surface of the elongate member 742. When the width 760 of the channel 750 is approximately equal to or less than the diameter of the elongate member 742 movement of the locking member 724 may become difficult or stop. The elongate member 742 may frictionally engage the side walls of the channel 750 to hold the first portion 702 of the handle 700 in a fixed position relative to the second portion 704 of the handle 700. When the locking member 724 has been radially displaced, the locking member 724 is in a locked configuration. It is contemplated that when the locking member 724 is in the locked configuration at least one of the first or second actuation members 746a, 746b may extend radially beyond an outer surface of the proximal gripping region 706. The actuation member 746a, 746b that extends radially beyond the outer surface of the proximal gripping region 706 may be the actuation member 746a, 746b that was not depressed.

It is contemplated that in either the locked or unlocked configuration, the first and the second actuation member 746a, 746b may extend radially beyond an outer surface of the intermediate region 710 of the first portion 702 of the handle 700. This may allow the user to locate and use the locking member 724 without requiring visual confirmation. In some cases, the amount of“hold” of the locking member 724 may be adjusted by increasing or decreasing the width 760 of the channel 750. For example, increasing the width 760 may decrease the hold while decreasing the width 760 may increase the hold. Alternatively, or additionally, spring features may be added within the channel 750 to increase the hold or to facilitate actuation of the locking member 724.

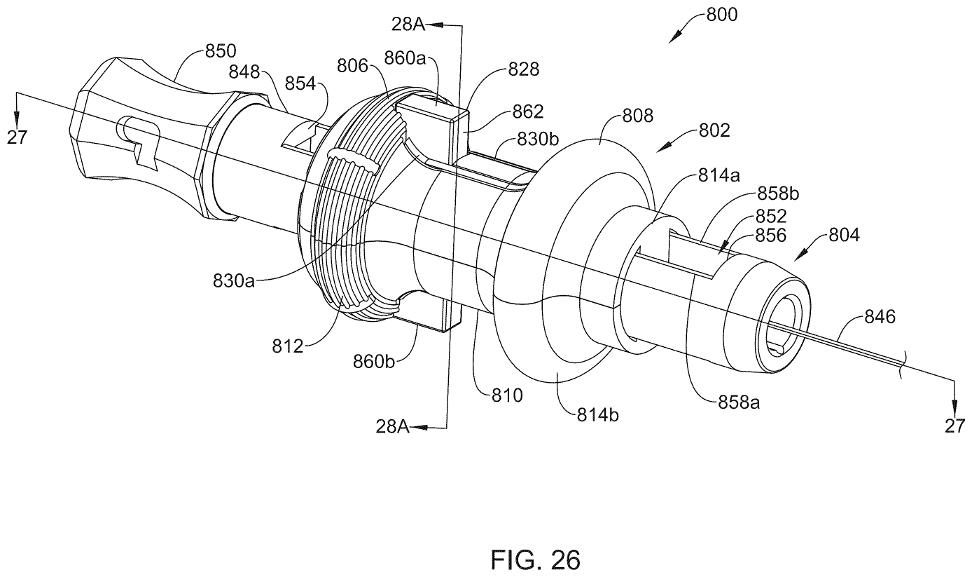

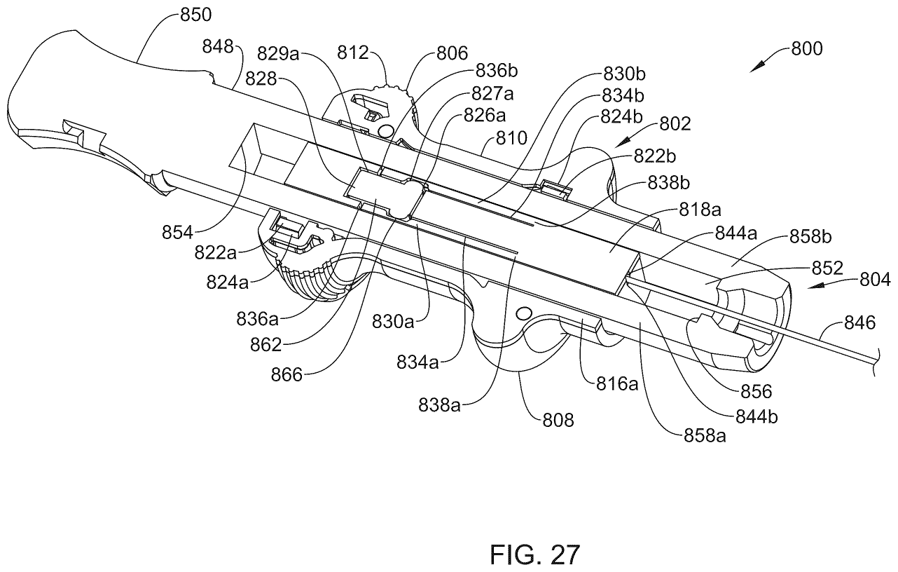

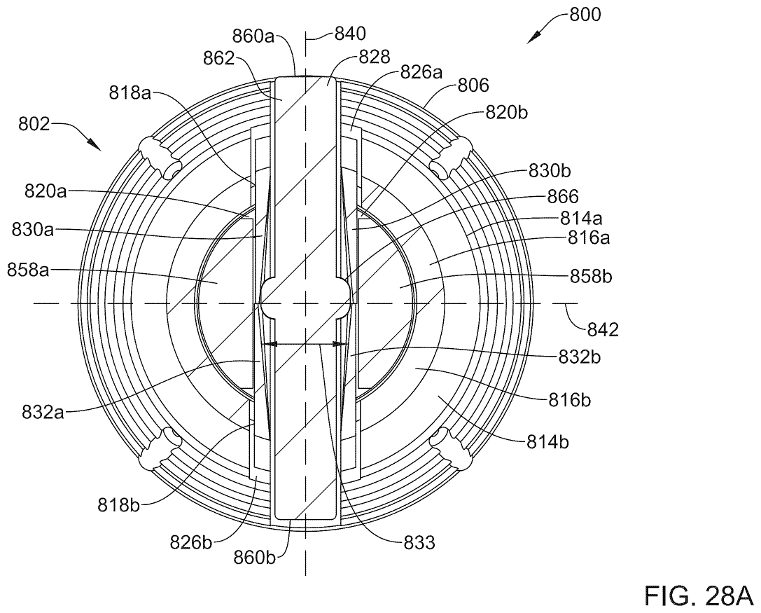

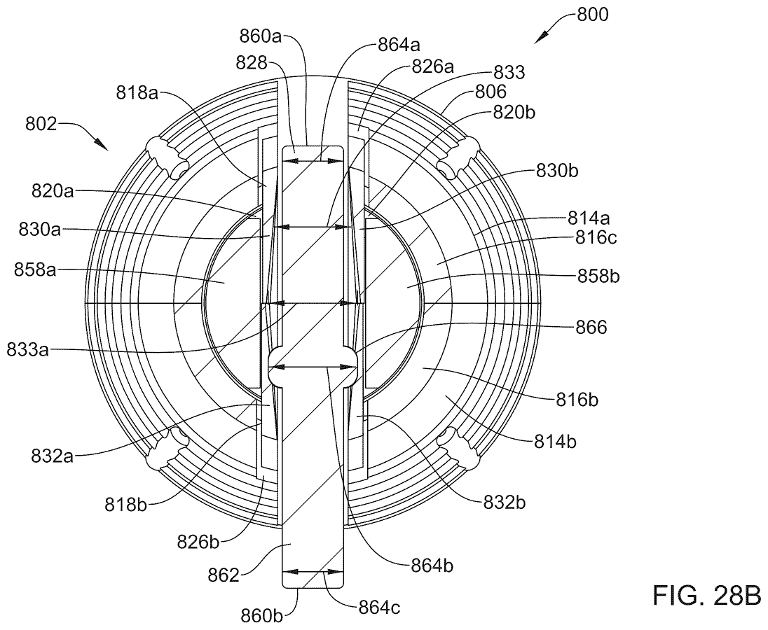

FIG. 26 is a schematic perspective view of another illustrative handle 800 that may be used to actuate and mechanically lock a wire filament of an accessory device. FIG. 27 is a cross-sectional view of the illustrative handle 800, taken at line 27-27 of FIG. 26. FIG. 28A is a cross-sectional view of the illustrative handle 800 of FIG. 26, taken at line 28A-28A with a locking member 828 in a first, unlocked, configuration. FIG. 28B is the cross-sectional view of FIG. 28A with the locking member 828 in a second, locked, configuration. The handle 800 may be configured to be coupled to a proximal end of the elongate tube (not explicitly shown) of an accessory device (e.g., similar in form and function to the elongate tube 26 and accessory device 14 described herein). The handle 800 may include a first portion 802 and a second portion 804. The first portion 802 of the handle 800 may be axially displaceable relative to the second portion 804 of the handle 800. For example, the first portion 802 may be proximally retracted relative to the second portion 804 to deflect the distal tip of the elongate tube in a first direction and the first portion 802 may be distally advanced relative to the second portion 804 to deflect the distal tip in a second direction opposite the first direction.

The first portion 802 may include a first, or proximal, gripping region 806 and a second, or distal, gripping region 808. The first and second gripping regions 806, 808 may have an enlarged cross-sectional profile relative to an intermediate region 810 disposed between the first and second gripping regions 806, 808. This may provide a region for the user to push or pull against to move the first portion 802 relative to the second portion 804. In some cases, one or both of the gripping regions 806, 808 may include texturing 812, such as raised ridges, grooves, bumps, or the like to increase the gripability of the first portion 802.

The first portion 802 may be formed from a first body member 814a and a second body member 814b. The first and second body members 814a, 814b may be fixedly or releasably coupled to one another over the second portion 804 of the handle 800. In some cases, the first and second body members 814a, 814b may be secured using mating latches 822a, 822b and tabs or ridges 824a, 824b, snap fits, friction fits, press-fits, or the like. Each body member 814a, 814b may include an arcuate outer portion 816a, 816b and a radially inward extending portion 818a, 818b. A wall thickness of the arcuate outer portion 816a, 816b may vary along a length thereof. Each of the arcuate outer portions 816a, 816b may have an arc length of approximately 180° such that collectively, the arcuate outer portions 816a, 816b form a complete or substantially complete ring. In some cases, a small gap may be present between adjacent surfaces of the first and second arcuate outer portions 816a, 816b. The radially inwardly extending portions 818a, 818b may extend towards a center of the first portion 802 of the handle 800. In some cases, the radially inwardly extending portions 818a, 818b may contact or meet in the middle. However, this is not required. In some cases, free ends of the radially inwardly extending portions 818a, 818b may be spaced from one another.

One or more gaps or openings 820a, 820b may be formed between the first and second body members 814a, 814b when they are assembled. These gaps 820a, 820b may extend from a proximal end to a distal end of the first and second body members 814a, 814b. The gaps 820a, 820b may be configured to receive the rails 858a, 858b of the second portion 804 to allow the first portion 802 to be axially displaced relative to the second portion 804. While not explicitly shown, in some configurations, the first portion 802 may further include an auxiliary lumen extending generally orthogonal to a longitudinal axis of the first portion 802. When so provided, the auxiliary lumen may extend from a laterally outward end to a laterally inward end and may be configured to receive an electrical connection member therethrough.

A distal end region of the radially inwardly extending portions 818a, 818b may each include a recess 844a, 844b extending proximally from the distal end of the first portion 802 of the handle 800. Collectively, the recesses 844a, 844b may form a lumen (similar to the lumen 326 described herein) for receiving a proximal end of a wire filament 846 therein. The lumen may include a helical region configured to facilitate retention of the wire filament 846 within the lumen. Additionally, or alternatively, the wire filament 846 may be secured using means such as, but not limited to, adhesives, friction fits, snap fits, press-fits, or the like. As the proximal end region of the wire filament 846 is secured to the first portion 802 of the handle 800 via the lumen, axial movement of the first portion 802 may be translated to the wire filament.

The radially inwardly extending portions 818a, 818b may extend into a slot 852 of the second portion 804 of the handle 800. At least a region of the radially inwardly extending portions 818a, 818b may include a slot or through hole 826a, 826b. Regions of the radially inwardly extending portions 818a, 818b free from the slots 826a, 826b may be substantially solid. The slots 826a, 826b may extend generally orthogonal to the longitudinal axis of the first portion 802 of the handle 800. The slots 826a, 826b may be aligned and configured to define a lumen extending across a height of the first portion 802 of the handle 800. The slots 826a, 826b may be configured to receive a locking member 828 therethrough, as will be described in more detail herein. In some cases, the slots 826a, 826b may be positioned within the intermediate region 810 of the first portion 802 of the handle 800. However, this is not required. The slots 826a, 826b may include a distal region 827a (not explicitly shown for the second body member 814b) having a width greater than the proximal region 829a of the slots 826a, 826b. The distal region 827a may be configured to receive a biasing region 866 of the locking member 828 therein.

The first radially inwardly extending portion 818a may further include a pair of deflectable arms 830a, 830b. The deflectable arms 830a, 830b may be configured to be biased radially outwards through movement of the locking member 828, as will be described in more detail herein. The deflectable arms 830a, 830b be formed in the radially inwardly extending portion 818a by positioning a slot or gap 834a, 834b radially inwards from a radially outward edge of the radially inwardly extending portion 818a. The deflectable arms 830a, 830b may include a free end 836a, 836b free from connection to the radially inwardly extending portion 818a and a connected end 838a, 838b connected to or formed as a single monolithic structure with the radially inwardly extending portion 818a. The gaps 834a, 834b may be non-linear.

Similarly, the second radially inwardly extending portion 818b may further include a pair of deflectable arms 832a, 832b. The deflectable arms 832a, 832b may be configured to be biased radially outwards through movement of the locking member 828, as will be described in more detail herein. While not explicitly shown, similar to the deflectable arms 830a, 830b of the first body member 814a, the deflectable arms 832a, 832b of the second body member 814b may be formed in the radially inwardly extending portion 818b by positioning a slot or gap radially inwards from a radially outward edge of the radially inwardly extending portion 818b. The deflectable arms 832a, 832b may include a free end free from connection to the radially inwardly extending portion 818a and a connected end connected to or formed as a single monolithic structure with the radially inwardly extending portion 818a. The free ends of the deflectable arms 832a, 832b of the second body member 814b may be axially aligned with the free ends 836a, 836b of the first body member 814a. The gaps may be non-linear.

A wall thickness of the arms 830a, 830b, 832a, 832b may vary along a height thereof in a region adjacent to the distal region 827a of the slots 826a, 826b. As used herein, the height of the arms 830a, 830b, 832a, 832b may extend in a direction generally parallel to a longitudinal axis 840 of the locking member 828. The wall thickness of the arms 830a, 830b, 832a, 832b may be measured in a direction extending generally orthogonal to the longitudinal axis 840 of the locking member and generally orthogonal to the longitudinal axis of the first portion 802 of the handle 800, as shown at axis 842. The wall thickness of the arms 830a, 830b, 832a, 832b may each decrease in a radially inwards direction in a sloped or tapered manner such that a distance 833 between opposing arms (e.g., between arm 830a and arm 830b and between arm 832a and arm 832b) is at a maximum distance 833a at a radially center point of the first portion 802 of the handle 800. It is contemplated that the wall thickness of the arms 830a, 830b, 832a, 832b may vary along an axial length (extending parallel to a longitudinal axis of the first portion 802 of the handle 800) disposed adjacent to the distal region 827a of the slots 826a, 826 and may be substantially uniform along other regions of the arms 830a, 830b, 832a, 832b.

The second portion 804 of the handle 800 may include a generally cylindrical body portion 848 including a gripping region 850 extending from a proximal end thereof. The gripping region 850 may be configured to provide a region for the clinician to hold the second portion 804 relative to the first portion 802 of the handle 800. The body portion 848 may include a slot 852 extending through a thickness or across a diameter thereof. The slot 852 may extend from a proximal end 854 to a distal end 856. On either side of the slot 852, the second portion of the handle 800 may include a first rail 858a and a second rail 858b. The first portion 802 of the handle 800 may be axially displaceable along the rails 858a, 858b with the radially inwardly extending portions 818a, 818b of the first portion 802 of the handle 800 axially displaceable within the slot 852.

The proximal end 854 of the slot 852 may be configured to create a mechanical stop when a proximal end of the radially inwardly extending portions 818a, 818b contacts the proximal end 854 of the slot 852 to limit proximal movement of the first portion 802 of the handle 800. Similarly, the distal end 856 of the slot 852 may be configured to create a mechanical stop when a distal end of the radially inwardly extending portions 818a, 818b contacts the distal end 856 of the slot 852 to limit distal movement of the first portion 802 of the handle 800. It is contemplated that the length of the slot 852 may be selected based on the desired proximal and/or distal movement of the first portion 802 (and the desired amount of bend at the distal tip).

The first portion 802 of the handle 800 may be assembled with the second portion 804 such that the body portion 848 (e.g., rails 858a, 858b) of the second portion 804 is disposed within the openings 820a, 820b formed by the first and second body members 814a, 814b of the first portion 802. The first portion 802 may be axially displaceable along an outer surface of the second portion 804.

The locking member 828 may extend through the slots 826a, 826b of the radially inward extending portions 818a, 818b of the first portion 802 of the handle 800 and through the slot 852 of the second portion 804 of the handle 800. The locking member 828 may extend from a first actuation member 860a and a second actuation member 860b each configured to be positioned exterior to outer surface of the first portion 802 of the handle 800. In some cases, the actuation members 860a, 860b may be radially outward surfaces of the locking member 828. Generally, the locking member 828 may be configured to be radially displaced to selectively lock the first portion 802 of the handle 800 relative to the second portion 804 of the handle 800. For example, the locking member 828 may be movable between a first unlocked configuration (FIG. 28A) where the first portion 802 of the handle 800 moves freely relative to the second portion 804 of the handle 800 and a second, locked configuration (FIG. 28B) wherein the first portion 802 of the handle 800 is fixed relative to the second portion 804 of the handle 800.

The locking member 828 may include a body member 862 extending between the first actuation member 860a and the second actuation member 860b. The body member 862 may be generally solid. The body member 862 may have a height extending from the first actuation member 860a to the second actuation member 860b. A central region (e.g., about halfway between the first actuation member 860a and the second actuation member 860b in the height direction) may have a width 864b that is greater than a width 864a, 864c of the body member 862 adjacent to the first actuation member 860a or the second actuation member 860b to define a biasing region 866. The biasing region 866 may have an axial length that is less than an axial length of the locking member 828. For example, the biasing region 866 may be sized and shaped to be disposed within the distal region 827 of the slots 826a, 826b of the first and second body members 814a, 814b. The width 864b of the biasing region 866 may be less than a width of the distance between opposing arms (e.g., between arm 830a and arm 830b and between arm 832a and arm 832b) at a radially center point of the first portion 802 of the handle 800.

When the locking member 828 is positioned such that the biasing region 866 is adjacent a radially center point of the first portion 802 of the handle 800, as shown in FIG. 28A, the first portion 802 of the handle 800 may be proximally or distally displaceable along the second portion 804 of the handle 800. Said differently, when the locking member 828 is positioned such that the biasing region 866 is adjacent a radially center point of the first portion 802 of the handle 800, the locking member 828 is in the unlocked configuration. In the unlocked configuration, the first and second actuation members 860a, 860b may be radially aligned with an outer surface of the proximal gripping region 806. However, this is not required.

When the distal tip has been actuated to the desired configuration, the user may depress either the first or the second actuation members 860a, 860b to radially displace the locking member 828. In the illustrated configuration of FIG. 28B, the first actuation member 860a has been pushed, depressed, or otherwise actuated. As the locking member 828 is actuated, the biasing region 866 slides along one of the pairs of arms 830a, 830b or 832a, 832b. In the illustrated embodiment, the biasing region 866 slides along the arms 832a, 832b of the second body member 814b. When the width 864b of the biasing region 866 is approximately equal to or greater than the distance 833 between the pair of arms 832a, 832b movement of the locking member 828 may become difficult or stop. The biasing region 866 may deflect the arms 832a, 832b radially outwards such that the radially outward surfaces of the arms 832a, 832b frictionally engage the side walls of the rails 858a, 858b to hold the first portion 802 of the handle 800 in a fixed position relative to the second portion 804 of the handle 800. When the locking member 828 has been radially displaced, the locking member 828 is in a locked configuration. It is contemplated that when the locking member 828 is in the locked configuration at least one of the first or second actuation members 860a, 860b may extend radially beyond an outer surface of the proximal gripping region 806. The actuation member 860a, 860b that extends radially beyond the outer surface of the proximal gripping region 806 may be the actuation member 860a, 860b that was not depressed.

It is contemplated that in either the locked or unlocked configuration, the first and the second actuation member 860a, 860b may extend radially beyond an outer surface of the intermediate region 810 of the first portion 802 of the handle 800. This may allow the user to locate and use the locking member 828 without requiring visual confirmation. In some cases, the amount of “hold” of the locking member 828 may be adjusted by increasing or decreasing the width 864b of the biasing region 866. For example, increasing the width 864b may increase the hold while decreasing the width 864b may decrease the hold.

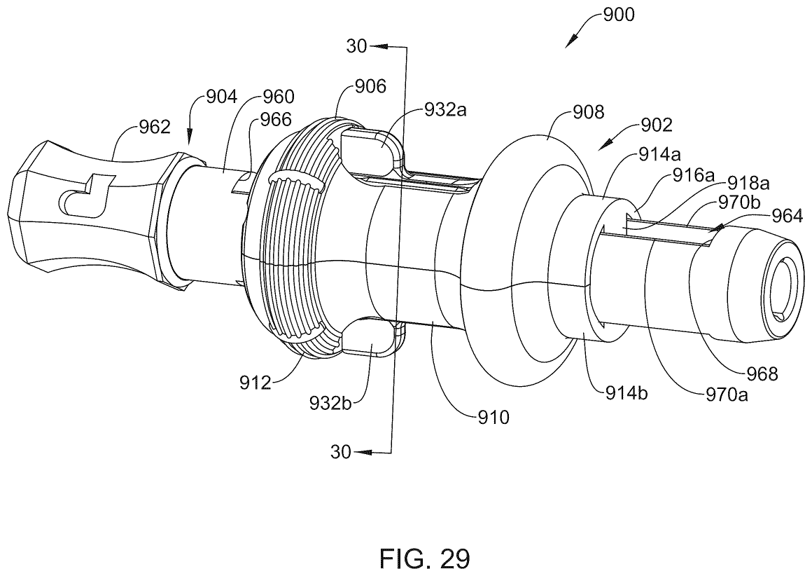

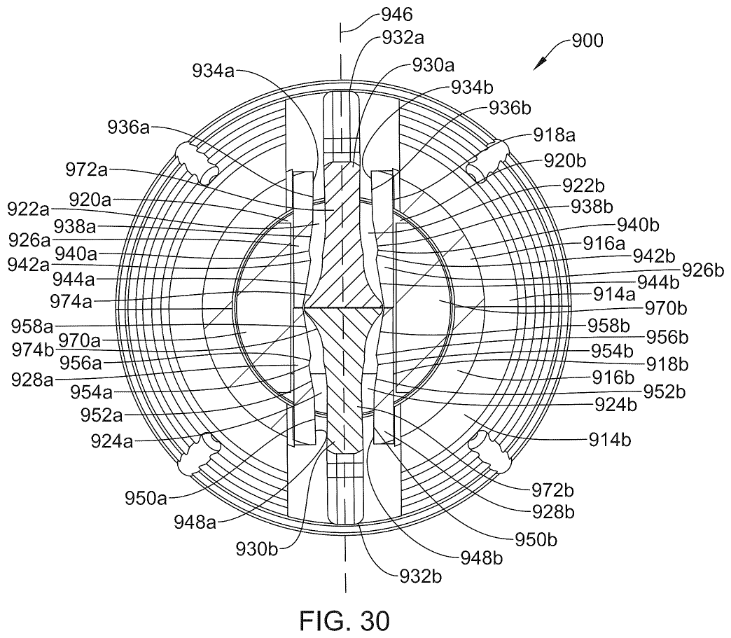

FIG. 29 is a schematic perspective view of another illustrative handle 900 that may be used to actuate and mechanically lock a wire filament of an accessory device. FIG. 30 is a cross-sectional view of the illustrative handle 900, taken at line 30-30 of FIG. 29. The handle 900 may be configured to be coupled to a proximal end of the elongate tube (not explicitly shown) of an accessory device (e.g., similar in form and function to the elongate tube 26 and accessory device 14 described herein). The handle 900 may include a first portion 902 and a second portion 904. The first portion 902 of the handle 900 may be axially displaceable relative to the second portion 904 of the handle 900. For example, the first portion 902 may be proximally retracted relative to the second portion 904 to deflect the distal tip of the elongate tube in a first direction and the first portion 902 may be distally advanced relative to the second portion 904 to deflect the distal tip in a second direction opposite the first direction.

The first portion 902 may include a first, or proximal, gripping region 906 and a second, or distal, gripping region 908. The first and second gripping regions 906, 908 may have an enlarged cross-sectional profile relative to an intermediate region 910 disposed between the first and second gripping regions 906, 908. This may provide a region for the user to push or pull against to move the first portion 902 relative to the second portion 904. In some cases, one or both of the gripping regions 906, 908 may include texturing 912, such as raised ridges, grooves, bumps, or the like to increase the gripability of the first portion 902.

The first portion 902 may be formed from a first body member 914a and a second body member 914b. The first and second body members 914a, 914b may be fixedly or releasably coupled to one another over the second portion 904 of the handle 900. In some cases, the first and second body members 914a, 914b may be secured using mating latches and tabs or ridges, snap fits, friction fits, press-fits, or the like. Each body member 914a, 914b may include an arcuate outer portion 916a, 916b and a radially inward extending portion 918a, 918b. A wall thickness of the arcuate outer portion 916a, 916b may vary along a length thereof. Each of the arcuate outer portions 916a, 916b may have an arc length of approximately 180° such that collectively, the arcuate outer portions 916a, 916b form a complete or substantially complete ring. In some cases, a small gap may be present between adjacent surfaces of the first and second arcuate outer portions 916a, 916b. The radially inwardly extending portions 918a, 918b may extend towards a center of the first portion 902 of the handle 900. In some cases, the radially inwardly extending portions 918a, 918b may contact or meet in the middle. However, this is not required. In some cases, free ends of the radially inwardly extending portions 918a, 918b may be spaced from one another.

One or more gaps or openings 920a, 920b may be formed between the first and second body members 914a, 914b when they are assembled. These gaps 920a, 920b may extend from a proximal end to a distal end of the first and second body members 914a, 914b. The gaps 920a, 920b may be configured to receive the rails 970a, 970b of the second portion 904 to allow the first portion 902 to be axially displaced relative to the second portion 904. While not explicitly shown, in some configurations, the first portion 902 may further include an auxiliary lumen extending generally orthogonal to a longitudinal axis of the first portion 902. When so provided, the auxiliary lumen may extend from a laterally outward end to a laterally inward end and may be configured to receive an electrical connection member therethrough.

A distal end region of the radially inwardly extending portions 918a, 918b may each include a recess (not explicitly shown) extending proximally from the distal end of the first portion 902 of the handle 900. Collectively, the recesses may form a lumen (similar to the lumen 326 described herein) for receiving a proximal end of a wire filament therein. The lumen may include a helical or angled region configured to facilitate retention of the wire filament within the lumen. Additionally, or alternatively, the wire filament may be secured using means such as, but not limited to, adhesives, friction fits, snap fits, press-fits, or the like. As the proximal end region of the wire filament is secured to the first portion 902 of the handle 900 via the lumen, axial movement of the first portion 902 may be translated to the wire filament.

The radially inwardly extending portions 918a, 918b may extend into a slot 964 of the second portion 904 of the handle 900. Each of the radially inwardly extending portions 918a, 918b may include a first slot 922a, 924a and a second slot 922b, 924b. The first pair of slots 922a, 922b may divide the first radially inwardly extending portion 918a into a pair of arms 926a, 926b and a locking member 930a. The locking member 930a may be disposed between the first arm 926a and the second arm 926b. Similarly, the second pair of slots 924a, 924b may divide the second radially inwardly extending portion 918b into a pair of arms 928a, 928b and a locking member 930b. The locking member 930b may be disposed between the first arm 928a and the second arm 928b. The locking members 930a, 930b may extend along less than entire axial length of the first and second body members 914a, 914b. Where the first and second body members 914a, 914b are free from the locking members 930a, 930b, the radially inwardly extending portion 918a, 918b may be substantially solid.

The locking members 930a, 930b may be radially displaceable relative to the first or second pairs of arms 926a, 926b, 928a, 928b to selectively lock the first portion 902 of the handle 900 relative to the second portion 904. For example, the first pair of deflectable arms 926a, 926b or the second pair of deflectable arms 928a, 928b may be configured to be biased radially outwards through movement of the locking members 930a, 930b, as will be described in more detail herein. An actuation member 932a, 932b may extend radially from the locking members 930a, 930b such that the actuation members 932a, 932b extend radially beyond an outer surface of the intermediate region 910 of the first portion 902 of the handle 900.

A radially inward surface 934a of the first arm 926a of the first radially inwardly extending portion 918a may be non-linear. Said differently, a wall thickness of the first arm 926a may vary along a height thereof to define a profiled radially inward surface 934a. For example, the first arm 926a may include a first region 936a which extends generally parallel to a longitudinal axis 946 of the locking members 930a, 930b. At a radially inward end of the first region 936a, the wall thickness of the first arm 926a may begin to reduce in a gradual tapered manner to define a second, sloped, region 938a which extends non-parallel to axis 946. At a radially inward end, or transition point 940a of the second, sloped, region 938a, the wall thickness of the first arm 926a may begin to increase in a gradual tapered manner to define a third, sloped, region 942a. The transition point 940a may define a generally “V” shaped lock notch. The transition point 940a may be configured to selectively receive a radially inward end of the locking member 930a therein. At a radially inward end of the third, sloped, region 942a, the wall thickness of the first arm 926a may begin to decrease in a gradual tapered manner to define a fourth, sloped, region 944a which terminates at the radially inward end of the first arm 926a.

Similarly, a radially inward surface 934b of the second arm 926b of the first radially inwardly extending portion 918a may be non-linear. Said differently, a wall thickness of the second arm 926b may vary along a height thereof to define a profiled radially inward surface 934b. For example, the second arm 926b may include a first region 936b which extends generally parallel to a longitudinal axis 946 of the locking members 930a, 930b. At a radially inward end of the first region 936b, the wall thickness of the second arm 926b may begin to reduce in a gradual tapered manner to define a second, sloped, region 938b which extends non-parallel to axis 946. At a radially inward end, or transition point, 940b of the second, sloped, region 938b, the wall thickness of the second arm 926b may begin to increase in a gradual tapered manner to define a third, sloped, region 942b. The transition point 940b may define a generally “V” shaped lock notch. The transition point 940b may be configured to selectively receive a radially inward end of the locking member 930a therein. At a radially inward end of the third, sloped, region 942b, the wall thickness of the second arm 926b may begin to decrease in a gradual tapered manner to define a fourth, sloped, region 944b which terminates at the radially inward end of the second arm 926b.

A radially inward surface 948a of the first arm 928a of the second radially inwardly extending portion 918b may be non-linear. Said differently, a wall thickness of the first arm 928a may vary along a height thereof to define a profiled radially inward surface 948a. For example, the first arm 928a may include a first region 950a which extends generally parallel to a longitudinal axis 946 of the locking members 930a, 930b. At a radially inward end of the first region 950a, the wall thickness of the first arm 928a may begin to reduce in a gradual tapered manner to define a second, sloped, region 952a which extends non-parallel to axis 946. At a radially inward end or transition point 954a, of the second, sloped, region 952a, the wall thickness of the first arm 928a may begin to increase in a gradual tapered manner to define a third, sloped, region 956a. The transition point 954a may define a generally “V” shaped lock notch. The transition point 954a may be configured to selectively receive a radially inward end of the locking member 930b therein. At a radially inward end of the third, sloped, region 956a, the wall thickness of the first arm 928a may begin to decrease in a gradual tapered manner to define a fourth, sloped, region 958a which terminates at the radially inward end of the first arm 928a.

Similarly, a radially inward surface 948b of the second arm 928b of the second radially inwardly extending portion 918b may be non-linear. Said differently, a wall thickness of the second arm 928b may vary along a height thereof to define a profiled radially inward surface 948b. For example, the second arm 928b may include a first region 950b which extends generally parallel to a longitudinal axis 946 of the locking members 930a, 930b. At a radially inward end of the first region 950b, the wall thickness of the second arm 928b may begin to reduce in a gradual tapered manner to define a second, sloped, region 952b which extends non-parallel to axis 946. At a radially inward end, or transition point, 954b of the second, sloped, region 952b, the wall thickness of the second arm 928b may begin to increase in a gradual tapered manner to define a third, sloped, region 956b. The transition point 954b may define a generally “V” shaped lock notch. The transition point 954b may be configured to selectively receive a radially inward end of the locking member 930b therein. At a radially inward end of the third, sloped, region 956b, the wall thickness of the second arm 928b may begin to decrease in a gradual tapered manner to define a fourth, sloped, region 958b which terminates at the radially inward end of the second arm 928b.

The second portion 904 of the handle 900 may include a generally cylindrical body portion 960 including a gripping region 962 extending from a proximal end thereof. The gripping region 962 may be configured to provide a region for the clinician to hold the second portion 904 relative to the first portion 902 of the handle 900. The body portion 960 may include a slot 964 extending through a thickness or across a diameter thereof. The slot 964 may extend from a proximal end 966 to a distal end 968. On either side of the slot 964, the second portion of the handle 900 may include a first rail 970a and a second rail 970b. The first portion 902 of the handle 900 may be axially displaceable along the rails 970a, 970b with the radially inwardly extending portions 918a, 918b of the first portion 902 of the handle 900 axially displaceable within the slot 964.

The proximal end 966 of the slot 964 may be configured to create a mechanical stop when a proximal end of the radially inwardly extending portions 918a, 918b contacts the proximal end 966 of the slot 964 to limit proximal movement of the first portion 902 of the handle 900. Similarly, the distal end 968 of the slot 964 may be configured to create a mechanical stop when a distal end of the radially inwardly extending portions 918a, 918b contacts the distal end 968 of the slot 964 to limit distal movement of the first portion 902 of the handle 900. It is contemplated that the length of the slot 964 may be selected based on the desired proximal and/or distal movement of the first portion 902 (and the desired amount of bend at the distal tip).

The first portion 902 of the handle 900 may be assembled with the second portion 904 such that the body portion 960 (e.g., rails 970a, 970b) of the second portion 904 is disposed within the openings 920a, 920b formed by the first and second body members 914a, 914b of the first portion 902. The first portion 902 may be axially displaceable along an outer surface of the second portion 904.

The first pair of arms 926a, 926b, the second pair of arms 928a, 928b, and the locking members 930a, 930b may extend through the slot 964 of the second portion 904 of the handle 900. Generally, one of the actuation members 932a, 932b may be actuated to actuate the locking members 930a, 930b to selectively lock the first portion 902 of the handle 900 relative to the second portion 904 of the handle 900. For example, the locking members 930a, 930b may be movable between a first unlocked configuration (FIG. 30) where the first portion 902 of the handle 900 moves freely relative to the second portion 904 of the handle 900 and a second, locked configuration (not explicitly shown) where the first portion 902 of the handle 900 is fixed relative to the second portion 904 of the handle 900.

The locking members 930a, 930b may each include a body member 972a, 972b. The body member 972a, 972b may be generally solid. The body member 972a, 972b may include a flared region 974a, 974b adjacent a radially inwards end thereof in which a width of the body member 972a, 972b increases towards the radially inwards end.

When the locking members 930a, 930b are positioned such that the flared regions 974a, 974b are adjacent a radially center point of the first portion 902 of the handle 900, as shown in FIG. 30, the first portion 902 of the handle 900 may be proximally or distally displaceable along the second portion 904 of the handle 900. In the unlocked configuration, the first and second actuation members 932a, 932b may be radially aligned with an outer surface of the proximal gripping region 906. However, this is not required.

When the distal tip has been actuated to the desired configuration, the user may depress either the first or the second actuation member 932a, 932b to radially displace the locking members 930a, 930b. For example, if the user depresses the first actuation member 932a, the locking member 930a may be radially displaced towards the second locking member 930b. The first locking member 930a may radially displace the second locking member 930b as it is radially displaced. The second locking member 930b may be radially displaced until the radially inward end thereof is positioned radially outward of the transition points 954a, 954b. The actuation member 932a may have a height such that when the radially outward surface thereof is aligned with the radially outward surface of the intermediate region 910 of the first portion 902 of the handle 900, the second locking member 930b has been radially displaced such that the radially inward end thereof is positioned radially outward of the transition points 954a, 954b. The third, sloped, surface 956a, 956b may guide the second locking member 930b into the transition points 954a, 954b. Once the user releases the first actuation member 932a, the first actuation member 932a and the first locking member 930a may return to an unbiased configuration where a radially inward end of the locking member 930a is generally aligned with the radially inward ends of the first and second arms 926a, 926b.

In the absence of an external biasing force, the transition points 954a, 954b may be configured to retain the radially inward end of the second locking member 930b therein. A wall thickness of the first and second arms 928a, 928b may be greater at the transition points 954a, 954b than a wall thickness adjacent a radially inward end of the first and second arms 928a, 928b. The width of the locking member 930b adjacent to the radially inward end thereof may be greater than the space between the first and second arms 928a, 928b adjacent to the transition points 954a, 954b. This may cause the first and second arms 928a, 928b to be deflected radially outwards such that the arms 928a, 928b frictionally engage the side walls of the rails 970a, 970b to hold the first portion 902 of the handle 900 in a fixed position relative to the second portion 904 of the handle 900. When at least one of the locking members 930a, 930b has been radially displaced, the locking member(s) 930a, 930b is in a locked configuration. To release the second locking member 930b and allow for axially movement of the first portion 902 of the handle 900 relative to the second portion 904, the user may depress the second actuation member 932b to disengage the radially inward end of the locking member 930b from the transition points 954a, 954b and return the second locking member 930b to an unbiased configuration where a radially inward end of the locking member 930b is generally aligned with the radially inward ends of the first and second arms 928a, 928b.

It is contemplated that the second actuation member 932b may be actuated to deflect the first locking member 930a into engagement with the transition points 940a, 940b in a similar manner to that described herein. It is contemplated that when one of the locking members 930a, 930b is in the locked configuration its respective actuation member 932a, 932b may extend radially beyond an outer surface of the proximal gripping region 906. The actuation member 932a, 932b that extends radially beyond the outer surface of the proximal gripping region 906 may be the actuation member 932a, 932b that was not depressed.

It is contemplated that in either the locked or unlocked configuration, the first and the second actuation member 932a, 932b may extend radially beyond an outer surface of the intermediate region 910 of the first portion 902 of the handle 900. This may allow the user to locate and use the locking members 930a, 930b without requiring visual confirmation. In some cases, the amount of “hold” of the locking members 930a, 930b may be adjusted by increasing or decreasing the width of the radially inward end of the locking members 930a, 930b. For example, increasing the width may increase the hold while decreasing the width may decrease the hold.

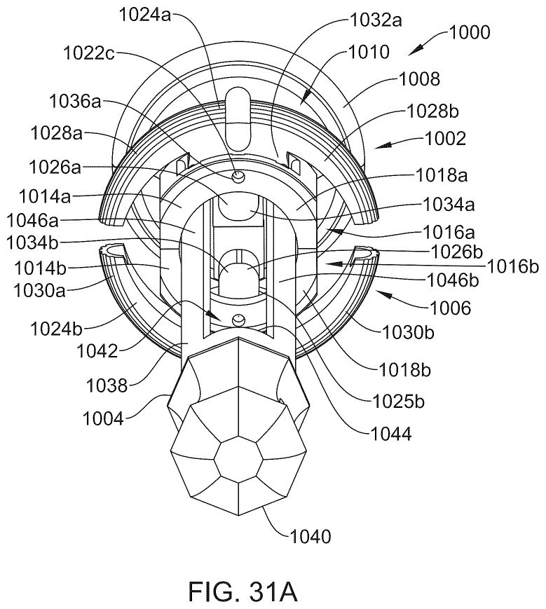

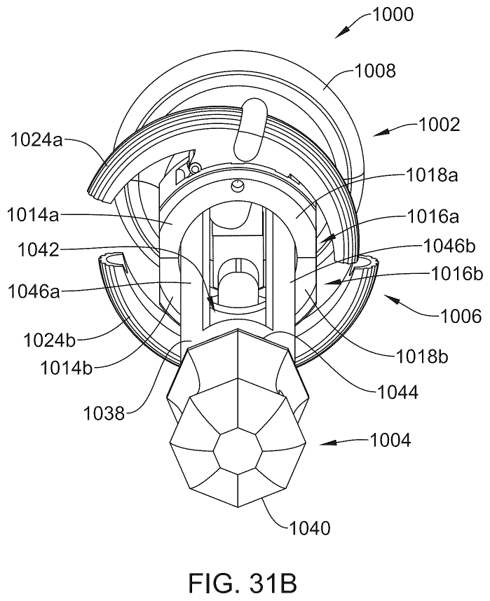

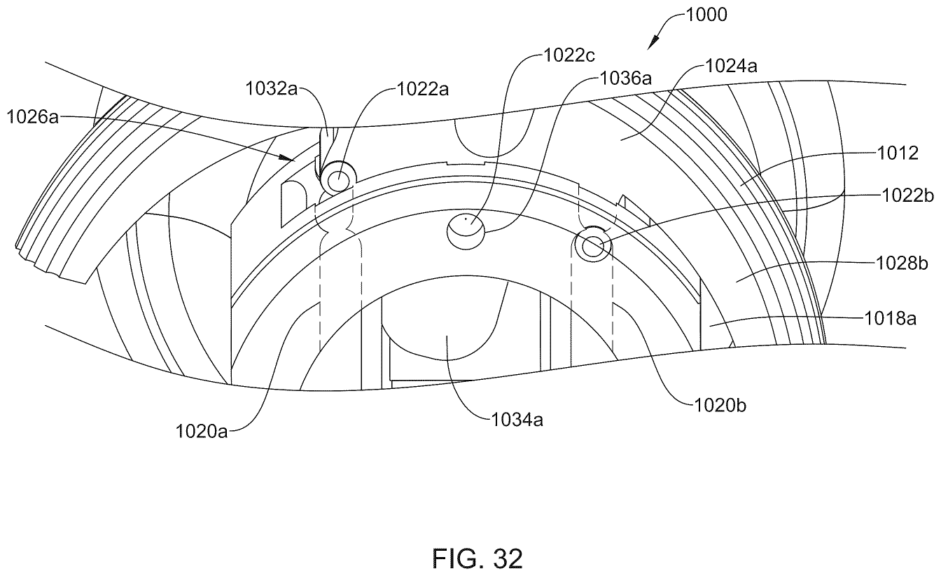

FIG. 31A is a schematic perspective view of another illustrative handle 1000 that may be used to actuate and mechanically lock a wire filament of an accessory device, in a first or unlocked configuration. FIG. 31B is a schematic perspective view of the illustrative handle 1000, in a second or locked configuration. FIG. 32 is a schematic partial cross-sectional view of the illustrative handle 1000 in the second or locked configuration. The handle 1000 may be configured to be coupled to a proximal end of the elongate tube (not explicitly shown) of an accessory device (e.g., similar in form and function to the elongate tube 26 and accessory device 14 described herein). The handle 1000 may include a first portion 1002 and a second portion 1004. The first portion 1002 of the handle 1000 may be axially displaceable relative to the second portion 1004 of the handle 1000. For example, the first portion 1002 may be proximally retracted relative to the second portion 1004 to deflect the distal tip of the elongate tube in a first direction and the first portion 1002 may be distally advanced relative to the second portion 1004 to deflect the distal tip in a second direction opposite the first direction.

The first portion 1002 may include a proximal toggle locking system 1006 and a distal, gripping region 1008. In some configurations, the toggle locking system 1006 may be distal to the gripping region 1008. The toggle locking system 1006 and the gripping region 1008 may have an enlarged cross-sectional profile relative to an intermediate region 1010 disposed between the proximal toggle locking system 1006 and the gripping region 1008. This may provide a region for the user to push or pull against to move the first portion 1002 relative to the second portion 1004. In some cases, one or both of the proximal toggle locking system 1006 and/or the gripping region 1008 may include texturing 1012 (see, for example, FIG. 32), such as raised ridges, grooves, bumps, or the like to increase the gripability of the first portion 1002.

The first portion 1002 may be formed from a first body member 1014a and a second body member 1014b. The first and second body members 1014a, 1014b may be fixedly or releasably coupled to one another over the second portion 1004 of the handle 1000. In some cases, the first and second body members 1014a, 1014b may be secured using mating latches and tabs or ridges, snap fits, friction fits, press-fits, or the like. Each body member 1014a, 1014b may extend from a proximal end region 1016a, 1016b to a distal end region (not explicitly shown). The proximal end regions 1016a, 1016b may include a generally arcuate retention member 1018a, 1018b. Referring more particularly to FIG. 32 which is an enlarged partial cross-sectional view of the retention member 1018a of the first body member 1014a, the retention member 1018a may include one or slots, grooves, or apertures 1020a, 1020b formed in a distal surface thereof. The slots 1020a, 1020b may be configured to selectively receive a mating pin 1022a, 1022b of an actuation member 1024a of the toggle locking system 1006, as will be described in more detail herein. While not explicitly shown, the retention member 1018b of the second body member 1014b may include slots, grooves, or apertures similar to slots 1020a, 1020b and configured to selectively receive a mating pin of an actuation member 1024b of the toggle locking system 1006.

The first and second body members 1014a, 1014b may include a slot or opening 1025b (not explicitly shown on the first body member 1014a) extending through a sidewall thereof. The slots 1025b may be each configured to receive a retention member 1026a, 1026b of the actuation members 1024a, 1024b therethrough.

The toggle locking system 1006 may include a first actuation member 1024a and a second actuation member 1024b. The first and second actuation members 1024a, 1024b may include generally arcuate body portions each extending in a curved manner from a first side region 1028a, 1030a to a second side region 1028b, 1030b. A retention member 1026a, 1026b may extend radially inwards from a central region of the first and second actuation members 1024a, 1024b. The retention members 1026a, 1026b may have a radially outward region 1032a having a first width and a radially inwards region 1034a, 1034b having a second width less than the first width. A first pin 1022a, a second pin 1022b, and a third pin 1022c may extend proximally from a proximal surface of the radially outward region 1032a of the retention members 1026a, 1026b. The first and second pins 1022a, 1022b may be configured to be selectively received within mating features of the first or second body members 1014a, 1014b to hold the first portion 1002 of the handle 1000 in a fixed position relative to the second portion 1004. The third pin 1022c may be received within a respective aperture 1036a of the retention member 1018a, 1018b. The first actuation member 1024a may be configured to pivot to about the third pin 1022c to move the toggle locking system 1006 between an unlocked configuration (FIG. 31A) and a locked configuration (FIG. 31B).

The second portion 1004 of the handle 1000 may include a generally cylindrical body portion 1038 including a gripping region 1040 extending from a proximal end thereof. The gripping region 1040 may be configured to provide a region for the clinician to hold the second portion 1004 relative to the first portion 1002 of the handle 1000. The body portion 1038 may include a slot 1042 extending through a thickness or across a diameter thereof. The slot 1042 may extend from a proximal end 1044 to a distal end (not explicitly shown). On either side of the slot 1042, the second portion of the handle 1000 may include a first rail 1046a and a second rail 1046b. The first portion 1002 of the handle 1000 may be axially displaceable along the rails 1046a, 1046b with the radially inwards region 1034a, 1034b of the retention members 1026a, 1026b extending into and axially displaceable within the slot 1042.

The proximal end 1044 of the slot 1042 may be configured to create a mechanical stop when a proximal end of the radially inwards region 1034a, 1034b of the retention members 1026a, 1026b contacts the proximal end 1044 of the slot 1042 to limit proximal movement of the first portion 1002 of the handle 1000. Similarly, the distal end of the slot 1042 may be configured to create a mechanical stop when a distal end region of the first and/or second body members 1014a, 1014b contacts the distal end of the slot 1042 to limit distal movement of the first portion 1002 of the handle 1000. It is contemplated that the length of the slot 1042 may be selected based on the desired proximal and/or distal movement of the first portion 1002 (and the desired amount of bend at the distal tip).

The first portion 1002 of the handle 1000 may be assembled with the second portion 1004 such that the body portion 1038 (e.g., rails 1046a, 1046b) of the second portion 1004 is disposed within or extend through the first and second body members 1014a, 1014b of the first portion 1002. The first portion 1002 may be axially displaceable along an outer surface of the second portion 1004.

When the distal tip has been actuated to the desired configuration, the user may depress either the first side region 1028a, 1030a or the second side region 1028b, 1030b of either the first or second actuation member 1024a, 1024b causing the depressed actuation member 1024a, 1024b to rotate about the central pin 1022c. In the illustrated configuration of FIG. 31B and FIG. 32, the second side region 1028b of the first actuation member 1024a has been depressed. As the actuation member 1024a rotates, the radially inwards region 1034a of the retention member 1026a rotates towards a surface of the first rail 1046a. The radially inwards region 1034a frictionally engages the side wall of the first rail 1046a to hold the first portion 1002 of the handle 1000 in a fixed position relative to the second portion 1004 of the handle 1000. Further, as the actuation member 1024a rotates, the second pin 1022 on the retention member 1026a is pushed into a slot 1020b (e.g., a mating slot) on the arcuate retention member 1018a. This may hold the distal end region of the elongate tube in the desired bent configuration without requiring the user to hold the first portion 1002 of the handle 1000 in the desired position. Returning the actuation member 1024a to the neutral position (FIG. 31A) (for example, by pushing on the opposing side region 1028a) may then release the frictional engagement between the radially inwards region 1034a and the rail 1046a to allow for free movement of the first portion 1002 relative to the second portion 1004 of the handle 1000. It is contemplated that in some cases, the user may move the first portion 1002 of the handle 1000 when the toggle lock system 1006 is actuated to a locked configuration with sufficient force. As actuating the toggle lock system 1006 radially displaces at least one of the first or second actuation members 1024a, 1024b, the user may not require visual confirmation to determine the position of the toggle position. For example, in the unlocked configuration, the toggle lock system 1006 has a generally circular configuration while in the unlocked configuration at least one end of one of the actuation members 1024a, 1024 is radially displaced breaking the generally circular structure.

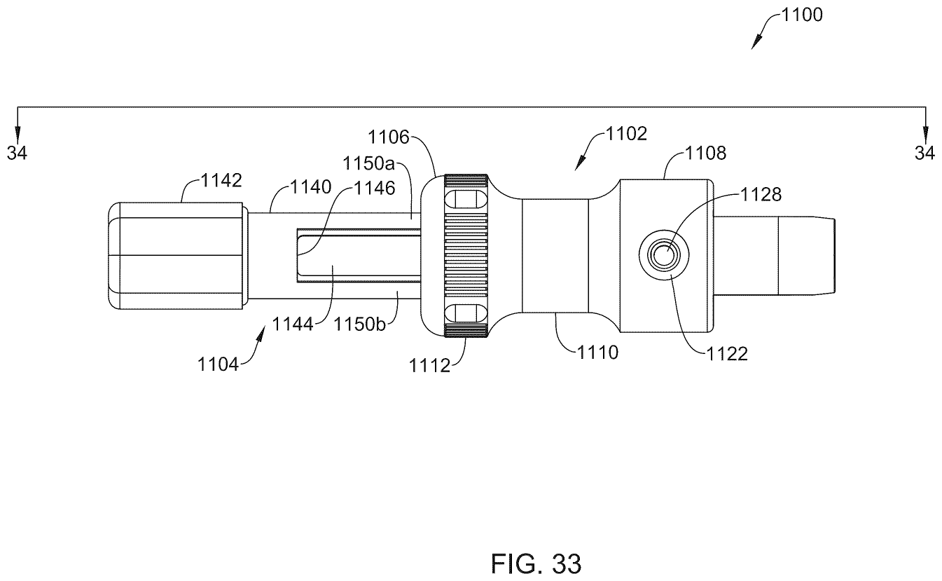

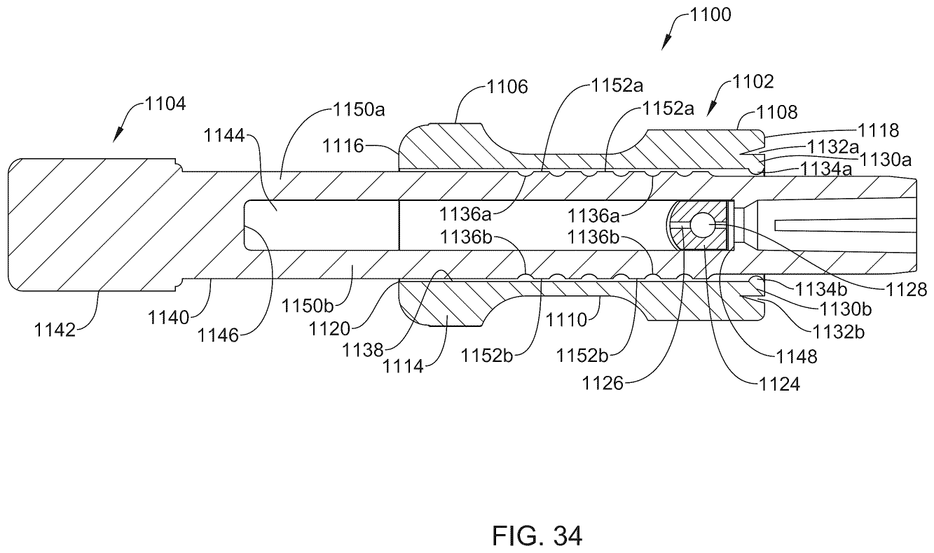

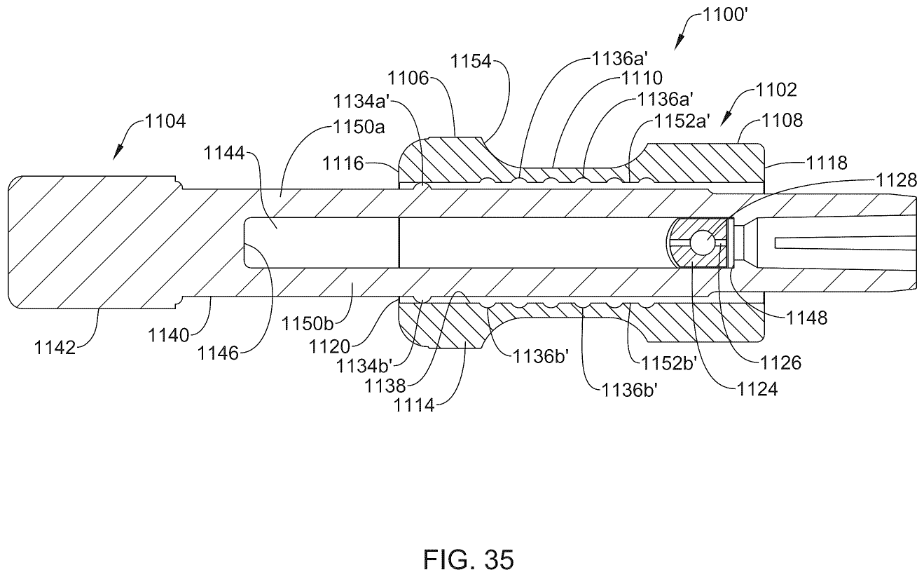

FIG. 33 is a side view of another illustrative handle 1100 that may be used to actuate and mechanically lock a wire filament of an accessory device. FIG. 34 is a cross-sectional view of the illustrative handle 1100, taken at line 34-34 of FIG. 33. The handle 1100 may be configured to be coupled to a proximal end of the elongate tube (not explicitly shown) of an accessory device (e.g., similar in form and function to the elongate tube 26 and accessory device 14 described herein). The handle 1100 may include a first portion 1102 and a second portion 1104. The first portion 1102 of the handle 1100 may be axially displaceable relative to the second portion 1104 of the handle 1100. For example, the first portion 1102 may be proximally retracted relative to the second portion 1104 to deflect the distal tip of the elongate tube in a first direction and the first portion 1102 may be distally advanced relative to the second portion 1104 to deflect the distal tip in a second direction opposite the first direction.

The first portion 1102 may include a first, or proximal, gripping region 1106 and a second, or distal, gripping region 1108. The first and second gripping regions 1106, 1108 may have an enlarged cross-sectional profile relative to an intermediate region 1110 disposed between the first and second gripping regions 1106, 1108. This may provide a region for the user to push or pull against to move the first portion 1102 relative to the second portion 1104. In some cases, one or both of the gripping regions 1106, 1108 may include texturing 1112, such as raised ridges, grooves, bumps, or the like to increase the gripability of the first portion 1102.

The first portion 1102 may include a generally tubular body member 1114 (e.g., a main body member) defining a lumen 1120 extending from a proximal 1116 end to a distal end 1118 thereof. The lumen 1120 may extend generally coaxially with a proximal end region of the elongate tube. The first portion 1102 may further include an auxiliary lumen 1122 extending generally orthogonal to the main lumen 1120. The auxiliary lumen 1122 may extend from a laterally outward end to a laterally inward end.

The auxiliary lumen 1122 may be configured to receive an electrical connection member (not explicitly shown) therethrough. An electrical connection member may include a threaded region configured to threadably engage a mating threaded region of a mounting member 1124. The mounting member 1124 may be fixedly disposed within the lumen 1120 of the body member 1114. The mounting member 1124 may include a first aperture 1126 extending from a proximal end to a distal end of the mounting member 1124. The first aperture 1126 may be configured to receive a proximal end region of a wire filament (similar in form and function to wire filament 130 described herein) therethrough. The mounting member 1124 may further include a second aperture 1128 extending generally orthogonal to the first aperture 1126 and parallel to the auxiliary lumen 1122 of the first portion 1102 of the handle 1100. The threaded region of the mounting member 1124 may be formed in the luminal wall of the second aperture 1128. The second aperture 1128 may be connected to the first aperture 1126 to allow a radially inward portion of the electrical connection member to contact the wire filament. As the proximal end region of the wire filament is secured to the first portion 1102 of the handle 1100 via the mounting member 1124 and the electrical connection member, movement of the first portion 1102 may be translated to the wire filament.

The body member 1114 may further include one or more cantilevered snaps 1130a, 1130b. The snaps 1130a, 1130b may be uniformly spaced from on another (e.g., spaced approximately 180° from one another). However, this is not required. While the illustrated configuration includes two snaps 1130a, 1130b, the body member 1114 may include fewer than two or more than two snaps 1130a, 1130b, as desired. The snaps 1130a, 1130b may each include a radially inwardly extending boss or protrusion 1134a, 1134b configured to be received within mating recesses 1136a, 1136b formed in the second portion 1104 of the handle 1100. For brevity not all of the recesses 1136a, 1136b have been identified with a reference number. While the protrusions 1134a, 1134b are illustrated as having a generally hemispherical cross-sectional shape, the protrusions 1134a, 1134b may take other shapes, such as, but not limited to “V”-shaped (e.g., generally triangular). The protrusions 1134a, 1134b may extend radially inwards to a greater extent that a remainder of the luminal wall 1138 of the body member 1114. The body member 1114 may have a gap or recess 1132a, 1132b positioned between the snaps 1130a, 1130b and an outer surface of the body member 1114. The gaps 1132a, 1132b may allow the snaps 1130a, 1130b to flex radially outwards as the first portion 1102 is moved proximally or distally along the second portion 1104. However, in some configurations, the gap(s) 1132a, 1132b may be omitted.

The second portion 1104 of the handle 1100 may include a generally cylindrical body portion 1140 including a gripping region 1142 extending from a proximal end thereof. The gripping region 1142 may be configured to provide a region for the clinician to hold the second portion 1104 relative to the first portion 1102 of the handle 1100. The body portion 1140 may include a slot 1144 extending through a thickness or across a diameter thereof. The slot 1144 may extend from a proximal end 1146 to a distal end 1148. On either side of the slot 1144, the second portion of the handle 1100 may include a first rail 1150a and a second rail 1150b. The first portion 1102 of the handle 1100 may be axially displaceable along the rails 1150a, 1150b with the mounting member 1124 of the first portion 1102 of the handle 1100 axially displaceable within the slot 1144.

The proximal end 1146 of the slot 1144 may be configured to create a mechanical stop when a proximal end of the mounting member 1124 contacts the proximal end 1146 of the slot 1144 to limit proximal movement of the first portion 1102 of the handle 1100. Similarly, the distal end 1148 of the slot 1144 may be configured to create a mechanical stop when a distal end of the mounting member 1124 contacts the distal end 1148 of the slot 1144 to limit distal movement of the first portion 1102 of the handle 1100. It is contemplated that the length of the slot 1144 may be selected based on the desired proximal and/or distal movement of the first portion 1102 (and the desired amount of bend at the distal tip).

The first portion 1102 of the handle 1100 may be assembled with the second portion 1104 such that the body portion 1140 (e.g., rails 1150a, 1150b) of the second portion 1104 is disposed within the central lumen 1120 of the first portion 1102. The first portion 1102 may be axially displaceable along an outer surface of the second portion 1104.

Each of the rails 1150a, 1150b may include a plurality of notches or recesses 1136a, 1136b formed in a radially outward surface thereof. The plurality of notches or recesses 1136a, 1136b may extend along an entire length of the rails 1150a, 1150b or less than an entire length, as desired. While the illustrated configuration has six recesses 1136a, 1136b on each rail 1150a, 1150b, the rails 1150a, 1150b may each include fewer than six or more than six recesses 1136a, 1136b, as desired. In some examples, the recess 1136a, 1136b may extend circumferentially along an entirety of an outer surface ofthe rails 1150a, 1150b or less than an entirety of the outer surface of the rails 1150a, 1150b, as desired. While the recesses 1136a, 1136b are illustrated as having a generally hemispherical cross-sectional shape, the recesses 1136a, 1136b may take other shapes, such as, but not limited to a “V”-shaped notch (e.g., generally triangular). It is contemplated that shape of the recesses 1136a, 1136b may conform to or mirror a shape of the protrusions 1134a, 1134b. The recesses 1136a, 1136b may be axially spaced by generally linearly extending portions 1152a, 1152b. For brevity, not all of the linearly extending portions 1152a, 1152b have identified with a reference number. A length of the linearly extending portions 1152a, 1152b may be increased or decreased to fine tune or adjust the control of the curvature of the distal tip. For example, decreasing a length of the linearly extending portions 1152a, 1152b may increase the control over the curvature of the distal tip.

As the user moves the first portion 1102 proximally or distally, the snaps 1130a, 1130b may flex radially outwards as the protrusions 1134a, 1134b move over the linearly extending portions 1152a, 1152b and drop with the recesses 1136a, 1136b as the protrusions 1134a, 1134b are adjacent thereto. For example, the snaps 1130a, 1130b may form an interference fit when the protrusions 1134a, 1134b are adjacent to the linearly extending portions 1152a, 1152b and may form a clearance fit when dropped into the recesses 1136a, 1136b. This may create a low-force lock when the protrusions 1134a, 1134b are aligned and positioned within mating recesses 1136a, 1136b. Thus, a user may not be required to acuate a locking mechanism when the distal tip of the elongate tube has been actuated to the desired configuration. The user may simply release the first portion 1102 and the mating protrusions 1134a, 1134b and recesses 1136a, 1136b will maintain the distal tip in the desired configuration. If the user desires to change a configuration of the distal tip, the user may simply apply a proximal or distal force to the first portion 1102. It is contemplated that the protrusions 1134a, 1134b entering and exiting the recesses 1136a, 1136b may create a tactile feedback sensation for the user as the first portion 1102 is axially displaced along the second portion 1104 of the handle 1100. It is contemplated that the symmetrical protrusions 1134a, 1134b and recesses 1136a, 1136b may allow for bidirectional locking and tactile feedback. In some cases, the cantilevered snaps 1130a, 1130b may include a cantilever feature to increase the flexibility of the feature and create a sharper “snap” when the protrusions 1134a, 1134b are seated within the recesses 1136a, 1136b. However, as noted above, it is not required that the snaps 1130a, 1130b be cantilevered.

In some configurations, the positions of the protrusions 1134a, 1134b and the recesses 1136a, 1136b may be reversed. FIG. 35 is a cross-sectional view of the handle 1100′ having an alternative arrangement of the protrusions 1134a, 1134b and the recesses 1136a, 1136b. However, other portions of the handle 1100′ may be similar in form and function to handle 1100 and are number similarly. In FIG. 35, the protrusions 1134a′, 1134b′ extend radially outwards from an outer surface of the rails 1150a, 1150b. The protrusions 1134a′, 1134b′ may be positioned anywhere between the proximal end 1146 of the slot 1144 and a distal end 1148 of the slot 1144. Further, the rails 1150a, 1150b may each include more than one protrusion 1134a′, 1134b′, as desired. The recesses 1136a′, 1136b′ may extend radially outwards from a luminal surface 1138 of the body member 1114. In some examples, the recesses 1136a′, 1136b′ may be formed as through holes extending from an outer surface 1154 of the body member 1114 to the luminal surface 1138 thereof. Forming the recesses 1136a′, 1136b′ as through holes may allow the first portion 1102 of the handle 1100′ to be injection molded. The protrusions 1134a′, 1134b′ and/or the recesses 1136a′, 1136b′ may be sized, shaped, and/or spaced (e.g., via a length of the linearly extending portions 1152a′, 1152b′) in a similar manner to that described above with respect to the protrusions 1134a, 1134b and the recesses 1136a, 1136b. Further, the handle 1100′ may be actuated and used in a manner similar to that described with respect to handle 1100.

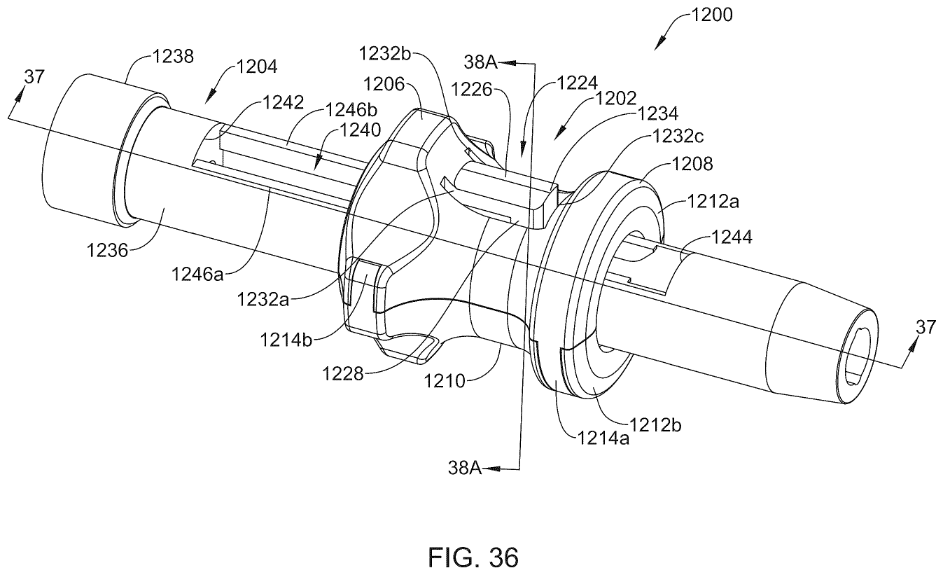

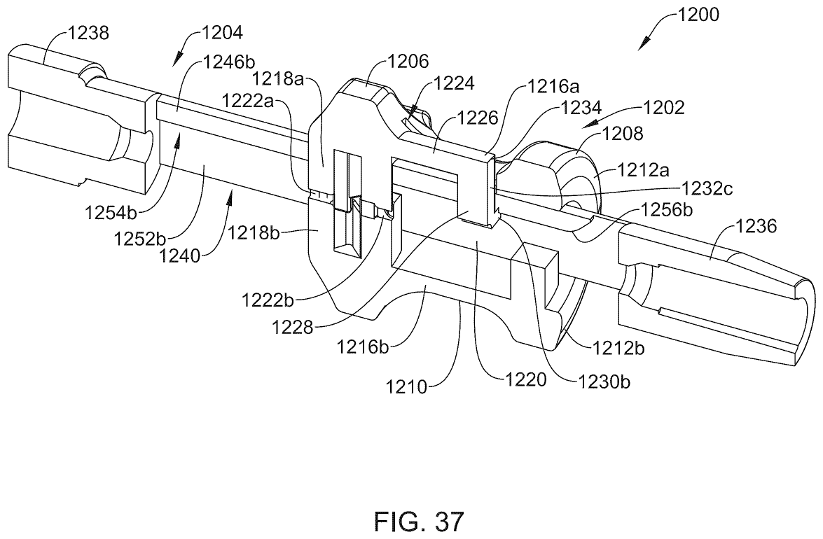

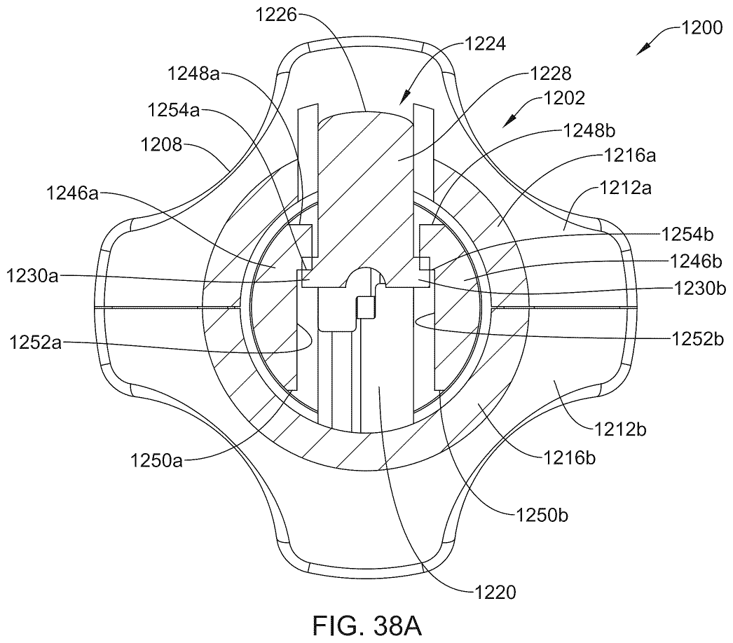

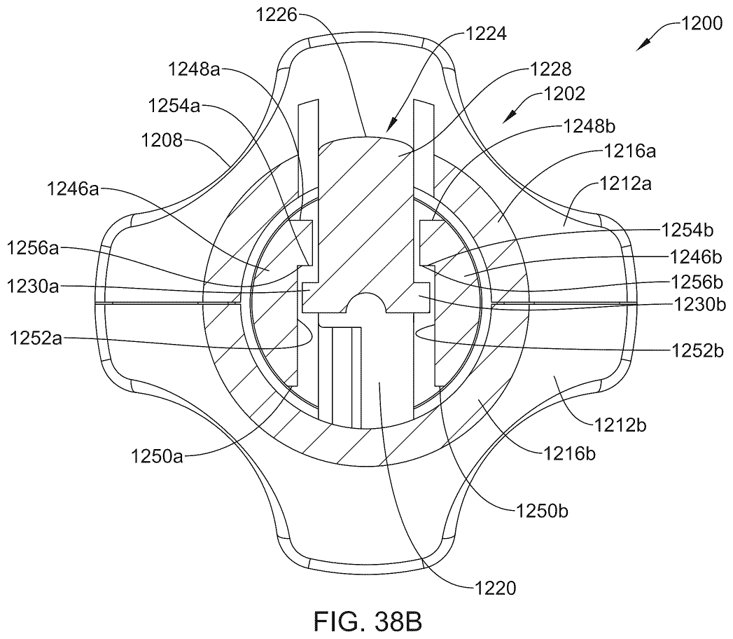

FIG. 36 is a schematic perspective view of another illustrative handle 1200 that may be used to actuate and mechanically lock a wire filament of an accessory device. FIG. 37 is a cross-sectional view of the illustrative handle 1200, taken at line 37-37 of FIG. 36. FIG. 38A is a cross-sectional view of the illustrative handle 1200, taken at line 38A-38A of FIG. 36. FIG. 38B is the cross-sectional view of FIG. 38A with the handle 1200 in an unlocked configuration. The handle 1200 may be configured to be coupled to a proximal end of the elongate tube (not explicitly shown) of an accessory device (e.g., similar in form and function to the elongate tube 26 and accessory device 14 described herein). The handle 1200 may include a first portion 1202 and a second portion 1204. The first portion 1202 of the handle 1200 may be axially displaceable relative to the second portion 1204 of the handle 1200. For example, the first portion 1202 may be proximally retracted relative to the second portion 1204 to deflect the distal tip of the elongate tube in a first direction and the first portion 1202 may be distally advanced relative to the second portion 1204 to deflect the distal tip in a second direction opposite the first direction.

The first portion 1202 may include a first, or proximal, gripping region 1206 and a second, or distal, gripping region 1208. The first and second gripping regions 1206, 1208 may have an enlarged cross-sectional profile relative to an intermediate region 1210 disposed between the first and second gripping regions 1206, 1208. This may provide a region for the user to push or pull against to move the first portion 1202 relative to the second portion 1204. In some cases, one or both of the gripping regions 1206, 1208 may include texturing, such as raised ridges, grooves, bumps, or the like to increase the gripability of the first portion 1202. In other examples, the outer surface of the proximal or distal gripping regions 1206, 1208 may undulate.

The first portion 1202 may be formed from a first body member 1212a and a second body member 1212b. The first and second body members 1212a, 1212b may be fixedly or releasably coupled to one another over the second portion 1204 of the handle 1200. In some cases, the first and second body members 1212a, 1212b may be secured using mating latches 1214a, 1214b and tabs or ridges (not explicitly shown), snap fits, friction fits, press-fits, or the like. Each body member 1212a, 1212b may include an arcuate outer portion 1216a, 1216b and a radially inward extending portion 1218a, 1218b. Each of the arcuate outer portions 1216a, 1216b may have an arc length of approximately 180° such that collectively, the arcuate outer portions 1216a, 1216b form a complete or substantially complete ring. In some cases, a small gap may be present between adjacent surfaces of the first and second arcuate outer portions 1216a, 1216b. The radially inwardly extending portions 1218a, 1218b may extend towards a center of the first portion 1202 of the handle 1200. In some cases, the radially inwardly extending portions 1218a, 1218b may contact or meet in the middle. However, this is not required. In some cases, free ends of the radially inwardly extending portions 1218a, 1218b may be spaced from one another. The radially inward extending portion 1218a, 1218b may have a length less than a length of the first and second body members 1212a, 1212b. In some configurations, the radially inward extending portion 1218a, 1218b may be positioned adjacent to a proximal end of the first and second body members 1212a, 1212b. However, this is not required.

An axially extending lumen 1220 may be formed between the first and second body members 1212a, 1212b when they are assembled. The lumen 1220 may extend from a proximal end to a distal end of the first and second body members 1212a, 1212b. The lumen 1220 may have a non-circular cross-sectional shape. Further, the cross-sectional shape may vary along a length of the lumen 1220. The lumen 1220 may be configured to receive the rails 1246a, 1246b of the second portion 1204 to allow the first portion 1202 to be axially displaced relative to the second portion 1204. While not explicitly shown, in some configurations, the first portion 1202 may further include an auxiliary lumen extending generally orthogonal to a longitudinal axis of the first portion 1202. When so provided, the auxiliary lumen may extend from a laterally outward end to a laterally inward end and may be configured to receive an electrical connection member therethrough.

A distal end region of the radially inwardly extending portions 1218a, 1218b may each include a recess 1222a, 1222b extending proximally from the distal end of the radially inwardly extending portions 1218a, 1218b. Collectively, the recesses 1222a, 1222b may form a lumen (similar to the lumen 326 described herein) for receiving a proximal end of a wire filament therein. The lumen may include a helical or angled region configured to facilitate retention of the wire filament within the lumen. Additionally, or alternatively, the wire filament may be secured using means such as, but not limited to, adhesives, friction fits, snap fits, press-fits, or the like. As the proximal end region of the wire filament is secured to the first portion 1202 of the handle 1200 via the lumen, axial movement of the first portion 1202 may be translated to the wire filament. The radially inwardly extending portions 1218a, 1218b may extend into a slot 1240 of the second portion 1204 of the handle 1200.

The first body member 1212a may further include a cantilevered snap member 1224. While the snap member 1224 is shown and described as a part of the first body member 1212a, in some configurations, the snap member 1224 may be a part of the second body member 1212b. The snap member 1224 may include an actuation member 1226, an arm 1228, and first and second retention members 1230a, 1230b. The actuation member 1226 may extend axially along the intermediate region 1210 of the first portion 1202. For example, the actuation member 1226 may extend distally from a connected end attached to or formed as a single monolithic structure with the proximal gripping region 1206 of the first body member 1212a. In other examples, the actuation member 1226 may extend proximally from the distal gripping region 1208. A first gap 1232a may extend axially along a first side of the actuation member 1226 and a second gap 1232b may extend axially along a second side of the actuation member 1226. A third gap 1232c may extend circumferentially adjacent to a free end 1234 of the actuation member 1226 and between the first and second gaps 1226a, 1226b. The gaps 1226a, 1226b, 1226c may allow the actuation member 1226 to be depressed to selectively unlock (and move) the first portion 1202 of the handle 1200 relative to the second portion 1204. The actuation member 1226 may extend radially beyond an outer surface of the intermediate region 1210 of the first portion 1202 to allow the user to locate and depress the actuation member 1226 without visual confirmation.

The arm 1228 may be coupled to or formed as single monolithic structure with the actuation member 1226. The arm 1228 may extend radially inwards from the free end 1234 of the actuation member 1226 and into the lumen 1220 of the first portion 1202 of the handle 1200. The first and second retention members 1230a, 1230b may extend laterally outwards from a free end of the arm 1228.

The second portion 1204 of the handle 1200 may include a generally cylindrical body portion 1236 including a gripping region 1238 extending from a proximal end thereof. The gripping region 1238 may be configured to provide a region for the clinician to hold the second portion 1204 relative to the first portion 1202 of the handle 1200. The body portion 1236 may include a slot 1240 extending through a thickness or across a diameter thereof. The slot 1240 may extend from a proximal end 1242 to a distal end 1244. On either side of the slot 1240, the second portion of the handle 1200 may include a first rail 1246a and a second rail 1246b. The first portion 1202 of the handle 1200 may be axially displaceable along the rails 1246a, 1246b with the radially inwardly extending portions 1218a, 1218b of the first portion 1202 of the handle 1200 axially displaceable within the slot 1240.

The proximal end 1242 of the slot 1240 may be configured to create a mechanical stop when a proximal end of the radially inwardly extending portions 1218a, 1218b contacts the proximal end 1242 of the slot 1240 to limit proximal movement of the first portion 1202 of the handle 1200. Similarly, the distal end 1244 of the slot 1240 may be configured to create a mechanical stop when a distal end of the radially inwardly extending portions 1218a, 1218b contacts the distal end 1244 of the slot 1240 to limit distal movement of the first portion 1202 of the handle 1200. It is contemplated that the length of the slot 1240 may be selected based on the desired proximal and/or distal movement of the first portion 1202 (and the desired amount of bend at the distal tip).

The first portion 1202 of the handle 1200 may be assembled with the second portion 1204 such that the body portion 1236 (e.g., rails 1246a, 1246b) of the second portion 1204 is disposed within the lumen 1220 formed by the first and second body members 1212a, 1212b of the first portion 1202. The first portion 1202 may be axially displaceable along an outer surface of the second portion 1204.

The rails 1246a, 1246b may extend from a first end 1248a, 1248b adjacent to the first body member 1012a to a second end 1250a, 1250b adjacent to the second body member 1012b. A laterally inward surface 1252a, 1252b of the rails 1246a, 1246b (e.g., surfaces facing the slot 1240) may include a step-wise transition 1254a, 1254b to define a ledge 1256a, 1256b. When the snap member 1224 is in an unbiased configuration, a radially outward surface of the retention members 1230a, 1230b may be configured to frictionally engage the ledges 1256a, 1256b, as shown in FIGS. 37 and 38A. The frictional engagement between the retention members 1230a, 1230b and the ledges 1256a, 1256b may hold the first portion 1202 of the handle 1200 in place relative to the second portion 1204. Said differently, the frictional engagement between the retention members 1230a, 1230b and the ledges 1256a, 1256b may prevent the first portion 1202 of the handle 1200 from being axially displaced relative to the second portion 1204.

When the user desires to deflect the distal tip of the elongate tube, the user may depress the actuation member 1226 of the snap member 1224 to flex the actuation member 1226 and move the arm 1228 and the retention members 1230a, 1230b radially inwards to an unlocked configuration, as shown in FIG. 38B. In the unlocked configuration, the retention members 1230a, 1230b move away from the ledges 1256a, 1256b to remove the frictional engagement therebetween. While the actuation member 1226 is depressed, the first portion 1202 of the handle 1200 may be freely axially displaced along the second portion 1204 of the handle 1200. When the distal tip is in the desired configuration, the user may release the snap member 1224 to re-engage the retention members 1230a, 1230b with the ledges 1256a, 1256b to lock the distal tip. The snap member 1224 may be biased to the radially outward configuration such that the snap member 1224 returns to the locked configuration without additional user interaction after releasing the snap member 1224. In some configurations, the ledges 1256a, 1256b of the rails 1246a, 1246b may be smooth or may include notches for tactile feedback.

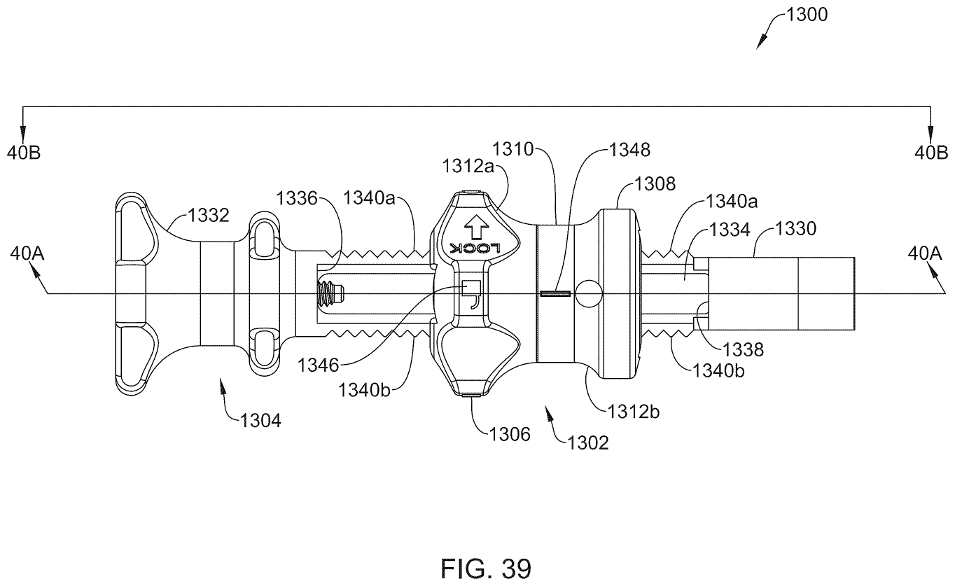

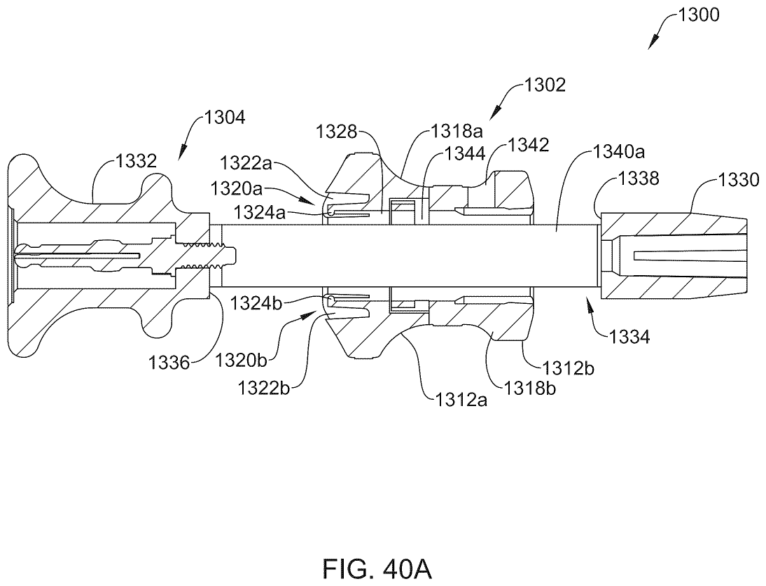

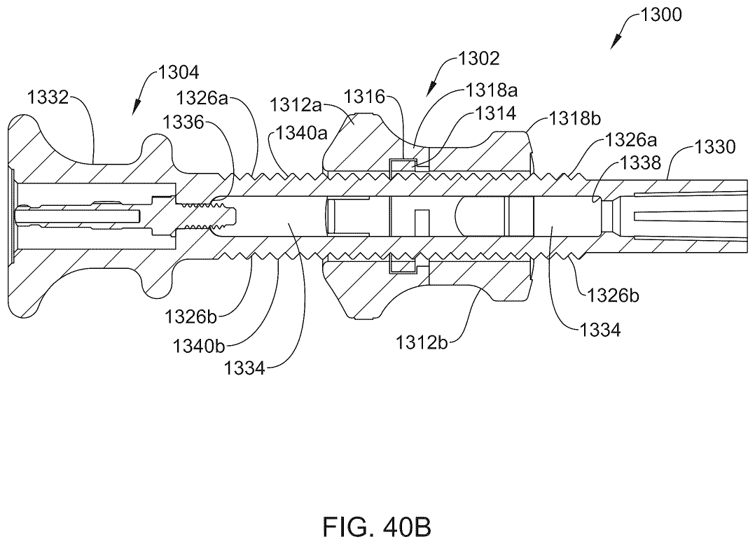

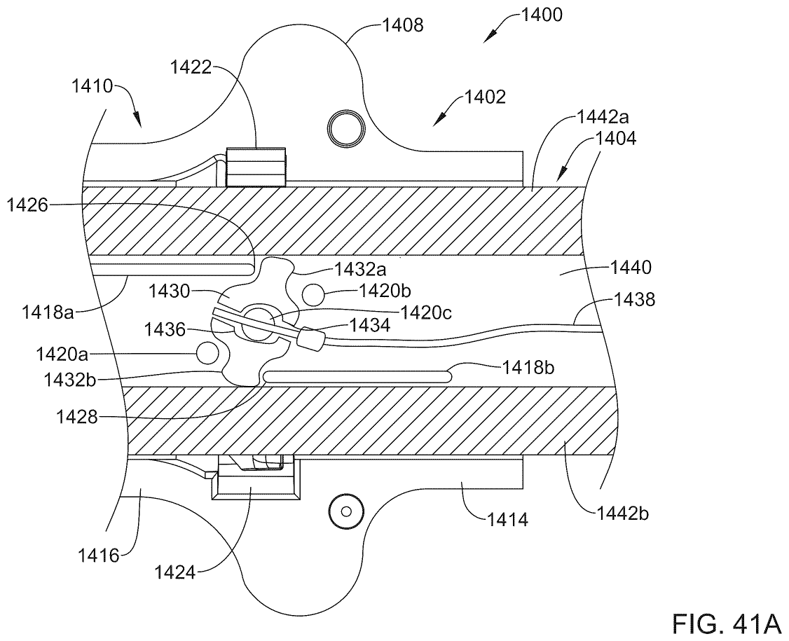

FIG. 39 is a schematic perspective view of another illustrative handle 1300 that may be used to actuate and mechanically lock a wire filament of an accessory device. FIG. 40A is a cross-sectional view of the illustrative handle 1300, taken at line 40A-40A of FIG. 39. FIG. 40B is a cross-sectional view of the illustrative handle 1300, taken at line 40B-40B of FIG. 39. The handle 1300 may be configured to be coupled to a proximal end of the elongate tube (not explicitly shown) of an accessory device (e.g., similar in form and function to the elongate tube 26 and accessory device 14 described herein). The handle 1300 may include a first portion 1302 and a second portion 1304. The first portion 1302 of the handle 1300 may be axially displaceable relative to the second portion 1304 of the handle 1300. For example, the first portion 1302 may be proximally retracted relative to the second portion 1304 to deflect the distal tip of the elongate tube in a first direction and the first portion 1302 may be distally advanced relative to the second portion 1304 to deflect the distal tip in a second direction opposite the first direction.

The first portion 1302 may include a first, or proximal, gripping region 1306 and a second, or distal, gripping region 1308. The first and second gripping regions 1306, 1308 may have an enlarged cross-sectional profile relative to an intermediate region 1310 disposed between the first and second gripping regions 1306, 1308. This may provide a region for the user to push or pull against to move the first portion 1302 relative to the second portion 1304. In some cases, one or both of the gripping regions 1306, 1308 may include texturing, such as raised ridges, grooves, bumps, or the like to increase the gripability of the first portion 1302. In other examples, the outer surface of the proximal or distal gripping regions 1306, 1308 may undulate.

The first portion 1302 may be formed from a first, or proximal, body member 1312a and a second, or distal, body member 1312b. The first and second body members 1312a, 1312b may be fixedly or releasably coupled to one another over the second portion 1304 of the handle 1300. In some cases, the first and second body members 1312a, 1312b may be secured such that the first body member 1312a is rotatable relative to the second body member 1312b. The coupling may be configured such that both the first and second body members 1312a, 1312b are axially displaceable when a pushing or pulling force is applied to either of the first or second body members 1312a, 1312b. In the illustrated embodiment, a protrusion 1314 of the second body member 1312b may be configured to be received with a recess 1316 of the first body member 1312a. The protrusion 1314 and recess 1316 may create a mechanical interlock the axial direction. Said differently, the sidewalls of the protrusion 1314 may engage the sidewalls of the recess 1316 such that axial movement of the first body member 1312a is transferred to the second body member 1312b and axial movement of the second body member 1312b is transferred to the first body member 1312a while allowing the first body member 1312a to be rotated relative to the second body member 1312b.

The first body member 1312a may include a generally annular outer wall 1318a. A thickness of the outer wall 1318a may vary along a length of the first body member 1312a. The recess 1316 may be formed in a luminal surface of the outer wall 1318a. The first body member 1312a may include one or more cantilevered snap members 1320a, 1320b. The snap members 1320a, 1320b may be uniformly spaced from on another (e.g., spaced approximately 180° from one another. The snap members 1320a, 1320b may be formed in a radially inwards portion of the first body member 1312a. In some cases, a gap or recess 1322a, 1322b may be positioned radially outward of the snap members 1320a, 1320b to allow the snap members 1320a, 1320b to flex radially outwards as the first portion 1302 is moved proximally or distally along the second portion 1304. However, in some configurations, the gap 1322a, 1322b may be omitted.

While the illustrated configuration includes two snap members 1320a, 1320b, the first body member 1312a may include fewer than two or more than two snap members 1320a, 1320b, as desired. The snap members 1320a, 1320b may each include a radially inwardly extending boss or protrusion 1324a, 1324b configured to be selectively received within mating recesses 1326a, 1326b formed in the second portion 1304 of the handle 1300. For brevity not all of the recesses 1326a, 1326b have identified with a reference number. While the protrusions 1324a, 1324b are illustrated as having a generally V”-shaped (e.g., generally triangular) cross-sectional shape, the protrusions 1324a, 1324b may take other shapes, such as, but not limited to, hemispherical. The protrusions 1324a, 1324b may extend radially inwards.

The second body member 1312b may include a generally annular outer wall 1318b. A thickness of the outer wall 1318b may vary along a length of the second body member 1312b. The protrusion 1314 may be a tab extending proximally of a distal end of the first body member 1312a. Collectively, the first and second body members 1312a, 1312b may define a lumen 1344 extending from a proximal end to a distal end of the first portion 1302 of the handle 1300. The lumen 1344 may extend generally coaxially with a proximal end region of the elongate tube. The second body member 1312b may further include an auxiliary lumen 1342 extending generally orthogonal to the main lumen 1344. The auxiliary lumen 1342 may extend from a laterally outward end to a laterally inward end.

The auxiliary lumen 1342 may be configured to receive an electrical connection member (not explicitly shown) therethrough. An electrical connection member may include a threaded region configured to threadably engage a mating threaded region of a mounting member (similar in form and function to mounting member 1124 described herein). The mounting member may be fixedly disposed within the lumen 1344 of the first portion 1302 of the handle 1300. The mounting member may include a first aperture extending from a proximal end to a distal end of the mounting member. The first aperture may be configured to receive a proximal end region of a wire filament (similar in form and function to wire filament 130 described herein) therethrough. The mounting member may further include a second aperture extending generally orthogonal to the first aperture and parallel to the auxiliary lumen 1342 of the first portion 1302 of the handle 1300. The threaded region of the mounting member may be formed in the luminal wall of the second aperture. The second aperture may be connected to the first aperture to allow a radially inward portion of the electrical connection member to contact the wire filament. As the proximal end region of the wire filament is secured to the first portion 1302 of the handle 1300 via the mounting member and the electrical connection member, movement of the first portion 1302 may be translated to the wire filament.

The second portion 1304 of the handle 1300 may include a generally cylindrical body portion 1330 including a gripping region 1332 extending from a proximal end thereof. The gripping region 1332 may be configured to provide a region for the clinician to hold the second portion 1304 relative to the first portion 1302 of the handle 1300. The body portion 1330 may include a slot 1334 extending through a thickness or across a diameter thereof. The slot 1334 may extend from a proximal end 1336 to a distal end 1338. On either side of the slot 1334, the second portion of the handle 1300 may include a first rail 1340a and a second rail 1340b. The first portion 1302 of the handle 1300 may be axially displaceable along the rails 1340a, 1340b with the mounting member of the first portion 1302 of the handle 1300 axially displaceable within the slot 1334.

The proximal end 1336 of the slot 1334 may be configured to create a mechanical stop when a proximal end of the mounting member contacts the proximal end 1336 of the slot 1334 to limit proximal movement of the first portion 1302 of the handle 1300. Similarly, the distal end 1338 of the slot 1334 may be configured to create a mechanical stop when a distal end of the mounting member contacts the distal end 1338 of the slot 1334 to limit distal movement of the first portion 1302 of the handle 1300. It is contemplated that the length of the slot 1334 may be selected based on the desired proximal and/or distal movement of the first portion 1302 (and the desired amount of bend at the distal tip).

The first portion 1302 of the handle 1300 may be assembled with the second portion 1304 such that the body portion 1330 (e.g., rails 1340a, 1340b) of the second portion 1304 is disposed within the central lumen 1344 of the first portion 1302. The first portion 1302 may be axially displaceable along an outer surface of the second portion 1304.

Each of the rails 1340a, 1340b may include a plurality of notches or recesses 1326a, 1326b formed in a radially outward surface thereof. The plurality of notches or recesses 1326a, 1326b may extend along an entire length of the rails 1340a, 1340b or less than an entire length, as desired. The rails 1340a, 1340b may each include any number of recesses 1326a, 1326b, as desired. In some examples, the recess 1326a, 1326b may extend circumferentially along an entirety of an outer surface of the rails 1340a, 1340b or less than an entirety of the outer surface of the rails 1340a, 1340b, as desired. While the recesses 1326a, 1326b are illustrated as having a generally “V”-shaped notch (e.g., generally triangular) cross-sectional shape, the recesses 1326a, 1326b may take other shapes, such as, but not limited to, hemispherical. It is contemplated that shape of the recesses 1326a, 1326b may conform to a shape of the protrusions 1324a, 1324b.

The first body member 1312a may be rotated between an unlocked configuration in which the snap members 1320a, 1320b are circumferentially offset from the recesses 1326a, 1326b. In this configuration, the first portion 1302 may be freely axially displaced along the second portion 1304 of the handle 1300 to deflect the distal tip of the elongate tube. When the distal tip of the elongate tube is in the desired configuration, the user may rotate the first body member 1312a of the first portion 1302 to bring the snap members 1320a, 1320b into circumferential alignment with the recesses 1326a, 1326b. This may position the protrusions 1324a, 1324b within the recesses 1326a, 1326b. This may create a low-force lock in which the protrusions 1324a, 1324b are aligned with and positioned within mating recesses 1326a, 1326b allowing the position of the first portion 1302 relative to the second portion 1304 to be held in place without requiring the user to maintain a grip on the first portion 1302. The first body portion 1312a may be rotated by about 90° to move the first portion 1302 between the locked and unlocked configuration. In some cases, the user may move the first portion 1302 without moving the first body member 1312a to the unlocked configuration. However, this may require a greater amount of force relative to the unlocked configuration. To unlock the first portion 1302, the first body member 1312a may be rotated again by about 90° to circumferentially offset the snap members 1320a, 1320b from the recesses 1326a, 1326b. It is contemplated that the first body member 1312a may be rotated in a single direction to move between the locked and unlocked configuration or may be rotated in opposite directions, as desired. For example, the first body member 1312a may be configured to rotate 360° relative to the second body member 1312b in either a clockwise or counterclockwise direction.

The first body member 1312a may include visual indicia 1346 to allow the user to quickly determine whether the first body member 1312a is in the locked or unlocked configuration. In some cases, the visual indicia 1346 may include a symbol or alphanumeric word or phrase. In some cases, the visual indicia 1346 may provide a visual indication regarding a rotation direction to lock or unlock the first portion 1302 of the handle. The visual indicia 1346 may be aligned with another visual indicia 1348 on the second body member 1312b to indicate to the user which visual indicia on the first body member 1312 is valid if more than one is provided.

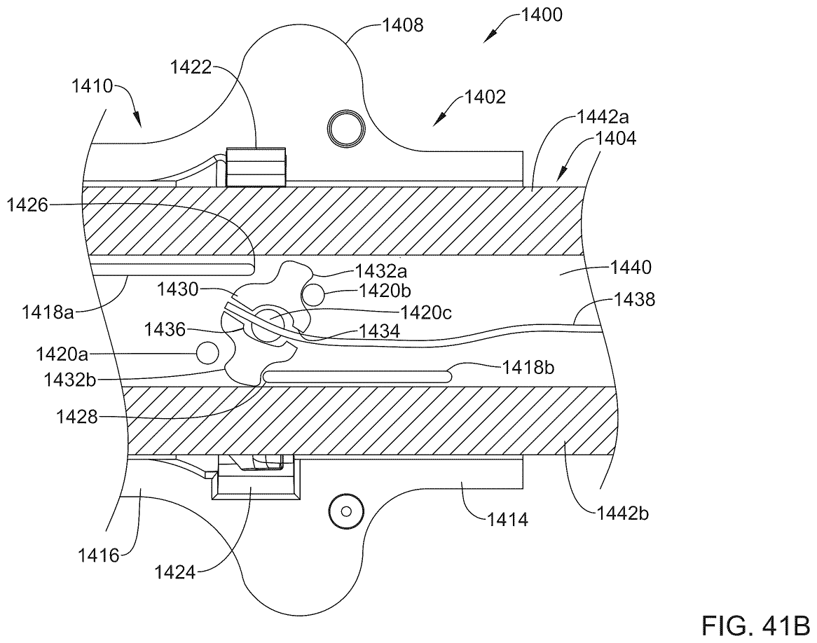

FIG. 41A is a partial cross-sectional view of another illustrative handle 1400 that may be used to actuate and mechanically lock a wire filament of an accessory device, in a first, or locked configuration. FIG. 41B is the illustrative handle 1400 of FIG. 41A in a second, or locked configuration. The handle 1400 may be configured to be coupled to a proximal end of the elongate tube (not explicitly shown) of an accessory device (e.g., similar in form and function to the elongate tube 26 and accessory device 14 described herein). The handle 1400 may include a first portion 1402 and a second portion 1404. The first portion 1402 of the handle 1400 may be axially displaceable relative to the second portion 1404 of the handle 1400. For example, the first portion 1402 may be proximally retracted relative to the second portion 1404 to deflect the distal tip of the elongate tube in a first direction and/or the first portion 1402 may be distally advanced relative to the second portion 1404 to deflect the distal tip in a second direction opposite the first direction.

The first portion 1402 may include a first, or proximal, gripping region, similar in form and function to other proximal gripping regions described herein (not explicitly shown) and a second, or distal, gripping region 1408. The first and second gripping regions 1408 may have an enlarged cross-sectional profile relative to an intermediate region 1410 of the first portion 1402. This may provide a region for the user to push or pull against to move the first portion 1402 relative to the second portion 1404. In some cases, one or both of the gripping regions 1408 may include texturing, such as raised ridges, grooves, bumps, or the like to increase the gripability of the first portion 1402.

The first portion 1402 may be formed from a first body member 1414 and a second body member (not explicitly shown). The second body member may have a same structure as the first body member 1414. The first body member 1414 may include an arcuate outer portion 1416 and a radially inward extending portion 1418a. The second body member may also include a radially inward extending portion 1418b. In some examples, the radially inward extending portions of the first or second body members 1414 may extend from a same body member. The first or second body member 1414 may further include one or more radially inward extending pins 1420a, 1420b, 1420c. A wall thickness of the arcuate outer portion 1416 may vary along a length thereof. The arcuate outer portion 1416 may have an arc length of approximately 180° such that collectively (e.g., when the first body member 1414 and the second body member are assembled), the arcuate outer portions 1416 form a complete or substantially complete ring (for example, similar to handles 300, 400 described herein). In some cases, a small gap may be present between adjacent surfaces of the first and second arcuate outer portions 1416. The first and second body members 1414 may be fixedly or releasably coupled to one another over the second portion 1404 of the handle 1400. In some cases, the first and second body members 1414 may be secured using mating latches 1422 and slots, tabs, or ridges 1424 snap fits, friction fits, press-fits, or the like. For example, latches 1422 on the first body member 1414 may be configured to engage mating slots, tabs or ridges (not explicitly shown) on the second body member and latches (not explicitly shown) on the second body member may be configured to engage mating slots, tabs or ridges 1424 on the first body member 1414. In some cases, the coupling mechanisms 1422, 1424 may be adjacent to the gripping regions 1408. However, this is not required. When so provided, the coupling mechanisms may be provided in other portions of the first or second body members 1414, as desired.

The radially inwardly extending portion 1418a may extend towards a center of the first portion 1402 of the handle 1400. The first radially extending portion 1418a may have a length that is less than a length of the first portion 1402 of the handle 1400. For example, the first radially extending portion 1418a may have a distal end 1426 that is proximal to a distal end of the first portion 1402 of the handle 1400. Similarly, the second radially extending portion 1418b may have a length that is less than a length of the first portion 1402 of the handle 1400. For example, the second radially extending portion 1418b may have a proximal end 1428 that is distal to a proximal end of the first portion 1402 of the handle 1400.

One or more gaps or openings (not explicitly shown) may be formed between the first and second body members 1414 when they are assembled. These gaps may extend from a proximal end to a distal end of the first and second body members 1414. The gaps may be configured to receive the rails 1442a, 1442b of the second portion 1404 to allow the first portion 1402 to be axially displaced relative to the second portion 1404. While not explicitly shown, in some configurations, the first portion 1402 may further include an auxiliary lumen extending generally orthogonal to a longitudinal axis of the first portion 1402. When so provided, the auxiliary lumen may extend from a laterally outward end to a laterally inward end and may be configured to receive an electrical connection member therethrough.

The first portion 1402 of the handle 1400 may further include a locking pivot member 1430. The locking pivot member 1430 may include a first radially extending arm 1432a and a second radially extending arm 1432b. The locking pivot member 1430 may be moved between a first locked configuration (FIG. 41A) in which an outer surface of the radially extending arms 1432a, 1432b are in contact with and engage the rails 1442a, 1442b and a second unlocked configuration (FIG. 41B) in which an outer surface of the radially extending arms 1432a, 1432b are spaced from and free from contact with the rails 1442a, 1442b. The locking pivot member 1430 may include a lumen 1434 extending therethrough. The lumen 1434 may be configured to receive a proximal portion of a wire filament 1438. The proximal portion of the wire filament 1438 may be secured to a centrally located pin 1420c to fixedly secure the wire filament 1438 to first portion 1402 of the handle 1400. The locking pivot member 1430 may be disposed over the central pin 1420c such that the central pin 1420c is disposed within an aperture 1436 of the locking pivot member 1430.

The second portion 1404 of the handle 1400 may include a generally cylindrical body portion (not explicitly shown) including a gripping region (not explicitly shown) extending from a proximal end thereof. The gripping region may be configured to provide a region for the clinician to hold the second portion 1404 relative to the first portion 1402 of the handle 1400. The body portion may include a slot 1440 extending through a thickness or across a diameter thereof. The slot 1440 may extend from a proximal end to a distal end. On either side of the slot 1440, the second portion of the handle 1400 may include a first rail 1442a and a second rail 1442b. The first portion 1402 of the handle 1400 may be axially displaceable along the rails 1442a, 1442b with the radially inwardly extending portions 1418a of the first portion 1402 of the handle 1400 axially displaceable within the slot 1440.

The proximal end of the slot 1440 may be configured to create a mechanical stop when a proximal end of the radially inwardly extending portion 1418a (or another region of the first portion 1402) contacts the proximal end of the slot 1440 to limit proximal movement of the first portion 1402 of the handle 1400. Similarly, the distal end of the slot 1440 may be configured to create a mechanical stop when a distal end of the radially inwardly extending portion 1418b (or another region of the first portion 1402) contacts the distal end of the slot 1440 to limit distal movement of the first portion 1402 of the handle 1400. It is contemplated that the length of the slot 1440 may be selected based on the desired proximal and/or distal movement of the first portion 1402 (and the desired amount of bend at the distal tip).

The first portion 1402 of the handle 1400 may be assembled with the second portion 1404 such that the body portion (e.g., rails 1442a, 1442b) of the second portion 1404 is disposed within the openings formed by the first and second body members 1414 of the first portion 1402. The first portion 1402 may be axially displaceable along an outer surface of the second portion 1404.

When movement or deflection of the distal tip of the elongate tube is desired, the user may distally advance or proximally retract the first portion 1402 of the handle 1400 relative to the second portion 1404 of the handle 1400. As the first portion 1402 is moved relative to the second portion 1404, the locking pivot member 1430 may contact one of the first or second radially inward extending portions 1418a, 1418b. For example, when the first portion 1402 is moved distally, the second arm 1432b may contact the proximal end 1428 of the second radially inward extending portion 1418b. The contact between the second arm 1432b and the proximal end 1428 of the second radially inward extending portion 1418b may cause the proximal end region of the wire filament 1438 to bend or flex and to rotate or pivot the locking pivot member 1430. As the locking pivot member 1430 is rotated the first and second arms 1432a, 1432b move away from the inner walls of the rails 1442a, 1442b allowing the frictional engagement therebetween to be removed and the first portion 1402 of the handle 1400 to move freely. When the distal actuation of the first portion 1402 is stopped (e.g., when the distal tip is in the desired configuration), the first portion 1402 may be released by the user. The proximal end region of the wire filament 1438 may be biased to a straight or linear configuration. Thus, in the absence of an applied force on the first portion 1402 of the handle 1400, the proximal end region of the wire filament 1438 may exert a biasing force on the locking pivot member 1430 as it returns or attempts to return to a straightened configuration causing the locking pivot member 1430 to pivot or rotate back to a locked configuration in which the surfaces of the arms 1432a, 1432b engage the rails 1442a, 1442b. This may hold the distal tip in the desired position without requiring the user to hold the first portion 1402 of the handle 1400.

Similarly, as a user proximally retracts the first portion 1402 of the handle 1400, the first arm 1432a may contact the distal end 1426 of the first radially inward extending portion 1418a. The contact between the first arm 1432a and the distal end 1426 of the first radially inward extending portion 1418a may cause the proximal end region of the wire filament 1438 to bend or flex and to rotate or pivot the locking pivot member 1430. As the locking pivot member 1430 is rotated the first and second arms 1432a, 1432b move away from the inner walls of the rails 1442a, 1442b allowing the frictional engagement therebetween to be removed and the first portion 1402 of the handle 1400 to move freely. Regardless of the direction of movement of the first portion 1402 of the handle 1400, friction between locking pivot member 1430 and the rails 1442a, 1442b may be minimized during movement of the first portion 1402 and maximized upon release of the first portion 1402.

The system 24, handles 100, 300, 400, 500, 600, 700, 800, 900, 1000, 1100, 1100′, 1200, 1300, 1400, locking mechanisms 200, 250, 360, 460 and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof, and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility is imparted into the system 24, handles 100, 300, 400, 500, 600, 700, 800, 900, 1000, 1100, 1100′, 1200, 1300, 1400, locking mechanisms 200, 250, 360, 460, and/or components thereof. For example, the system 24, handles 100, 300, 400, 500, 600, 700, 800, 900, 1000, 1100, 1100′, 1200, 1300, 1400, locking mechanisms 200, 250, 360, 460, and/or portions thereof may be made of a material that does not substantially distort the image and create substantial artifacts (i.e., gaps in the image). Certain ferromagnetic materials, for example, may not be suitable because they may create artifacts in an MRI image. The system 24, handles 100, 300, 400, 500, 600, 700, 800, 900, 1000, 1100, 1100′, 1200, 1300, 1400, locking mechanisms 200, 250, 360, 460, and/or components thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A medical device system comprising:
a handle having a first portion and a second portion, the first portion being axially displaceable relative to the second portion, the first portion comprising:
a body member having a central lumen extending therethrough;
one or more snap members extending from the body member, each snap member including a radially inwardly extending protrusion; and
wherein the second portion includes a plurality of recesses configured to selectively receive the radially inwardly extending protrusions of the one or more snap members.

2. The medical device system of claim 1, wherein the one or more snap members includes a first snap member and a second snap member.

3. The medical device system of claim 2, wherein each of the first and second snap members includes a gap positioned radially outward thereof.

4. The medical device system of claim 1, wherein the protrusions have a V-shaped cross-sectional shape.

5. The medical device system of claim 1, wherein the protrusions have a hemispherical cross-sectional shape.

6. The medical device system of claim 1, wherein the second portion includes a slot extending through a thickness thereof and a first rail and a second rail positioned on opposing sides of the slot.

7. The medical device system of claim 6, wherein the plurality of recesses is formed in the first and second rails.

8. The medical device system of claim 7, wherein the plurality of recesses extend along an entire length of the first and second rails.

9. The medical device system of claim 1, wherein the first portion is rotatable between a locked configuration and an unlocked configuration.

10. The medical device system of claim 9, wherein in the unlocked configuration, the one or more snap members are circumferentially offset from the plurality of recesses.

11. A medical device system comprising:
a handle having a first portion and a second portion, the first portion being axially displaceable relative to the second portion, the first portion comprising:
a proximal body member and a distal body member;
wherein the proximal body member is rotatable relative to the distal body member; and
wherein the proximal and distal body members are axially coupled such that axial movement of one of the proximal or distal body members causes axial movement of the other of the proximal or distal body members.

12. The medical device system of claim 11, wherein the proximal body member includes a recess and the distal body member includes a protrusion configured to be received within the recess.

13. The medical device system of claim 11, wherein the proximal body member includes one or more cantilevered snap members.

14. The medical device system of claim 13, wherein the second portion includes a plurality of recesses configured to selectively receive protrusions of the one or more cantilevered snap members.

15. The medical device system of claim 14, wherein the proximal body member is rotatable between a locked configuration where the protrusions are received within the plurality of recesses and an unlocked configuration where the protrusions are circumferentially offset from the plurality of recesses.

16. A medical device system comprising:
an elongate tube having a proximal end and a distal end;
a wire filament extending through the elongate tube;
a handle coupled to the proximal end of the elongate tube, the handle including:
a first portion and a second portion, the first portion being axially displaceable relative to the second portion;
wherein the first portion includes one or more snap members; and
wherein the second portion includes a plurality of recesses configured to selectively receive protrusions of the one or more snap members.

17. The medical device system of claim 16, wherein the first portion includes a proximal body member and a distal body member.

18. The medical device system of claim 17, wherein the proximal body member is rotatable relative to the distal body member.

19. The medical device system of claim 18, wherein the proximal and distal body members are axially coupled.

20. The medical device system of claim 16, wherein the first portion is rotatable between a locked configuration where the protrusions are received within the plurality of recesses and an unlocked configuration where the protrusions are circumferentially offset from the plurality of recesses.
